(19) United States
(12) Patent Application Publication
(10) Pub. No.: US 2024/0116950 A1
(43) Pub. Date: Apr. 11, 2024

(54) INHIBITORS OF KRAS(G12D)

(71) Applicant: Ranok Therapeutics (Hangzhou) Co. Ltd., Hangzhou (CN)

(72) Inventors: Weiwen Ying, Lexington, MA (US); Chenghao Ying, Shanghai (CN); Kevin P. Foley, Waltham, MA (US); Zhiyong Wang, Hangzhou (CN); Wei Yin, Lianyungang (CN); Liang Ma, Lianyungang (CN); Guoqiang Wang, Hangzhou (CN); Jinhua Li, Hangzhou (CN); Yaya Wang, Shanghai (CN); Yan Dai, Hangzhou (CN); Thomas Prince, Danville, PA (US)

(21) Appl. No.: 18/244,007

(22) Filed: Sep. 8, 2023

(30) Foreign Application Priority Data
Sep. 9, 2022 (WO) PCT/CN2022/118115

(51) Int. Cl. C07D 519/00; A61P 35/00

(52) U.S. Cl. CPC C07D 519/00; A61P 35/00

(57) ABSTRACT

Provided are small molecule inhibitors of the KRAS(G12D) mutant oncoprotein having the structural formula:

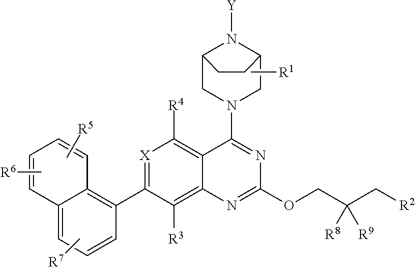

and pharmaceutically acceptable salts and compositions thereof, which are useful for treating cancers and related conditions.

### RELATED APPLICATIONS

This application claims the benefit of priority to International Application No. PCT/CN2022/118115, filed Sep. 9, 2022, the entire contents of which are incorporated herein by reference.

### BACKGROUND

Discovered as a human oncogene in the early 1980s, the Kirsten rat sarcoma virus homolog (KRAS) gene encodes a monomeric small 21 kDa GTPase that has long been an elusive cancer drug target (Chang et al., PNAS, 1982, 79:4848-52; McCoy et al., Nature, 1983, 302:79-8). KRAS functions as a molecular switch for promoting cell growth by cycling between GTP- and GDP-bound states. In the GTP-bound state, KRAS signals for growth through the RAF-MAPK and PI3K-AKT-MTOR pathways. KRAS subsequently hydrolyzes GTP to GDP with the aid of GTPase activating proteins (GAPs). This GDP-bound state switches “off” KRAS pro-growth signaling. KRAS can then be switched back “on” by GDP to GTP exchange through the aid of guanine nucleotide exchange factors, such as SOS1 (Cox and Der, Small GTPases, 2010, 1:2-27; Kerk et al., Nat Rev Cancer, 2021, 21:510-525). Preventing this exchange by locking KRAS in the GDP-bound state is a practical method for inhibiting its growth promoting activity.

The human KRAS gene is encoded on Chromosome 12p12.1 and is among the most frequently mutated genes in human cancers (Pylayeva-Gupta et al., Nat Rev Cancer, 2011, 11:761-774). Mutations that prevent GTP-hydrolysis lock KRAS in the active GTP-bound state and reprogram cells for perpetual proliferation. KRAS mutated from glycine (G) at the 12th codon to aspartate (D) creates a chronically active KRAS(G12D) oncoprotein, the gene for which is observed in 6.8% of cancers cases as analyzed by next-generation sequencing (Zhou et al., Pathol Oncol Res, 2020, 26:2835-2837). In tumor type-specific studies, KRAS(G12D) is associated with poor clinical outcomes and observed in 17% of lung, 14.3% of colorectal, and 48% of pancreatic tumors (Aredo et al., Lung Cancer, 2019, 133:144-150; Olmedillas-López et al., World J Gastroenterol, 2017, 23(39): 7087-709; Miglio et al., Pathol Res Pract, 2014, 210:307-11; Gou et al., Br J Cancer, 2020, 22:857-867), among other cancers. Historically, oncogenic KRAS mutants have been considered undruggable (McCormick F, Biochem J, 2019, 476:356-74), however the discovery of an allosteric pocket in GDP-bound KRAS has allowed the search for small molecule inhibitors (Ostrem et al., Nature, 2013, 503: 548-51). The G12D mutation moreover provides a unique chemical moiety-binding space due to the encoding of an acidic amino acid residue (D) in place of a small flexible amino acid residue possessing only a hydrogen side chain (G). This alteration of the KRAS protein structure provides a unique space that may be targeted with small molecules drugs that specifically inhibit KRAS(G12D) oncogenic activity. It is therefore desirable to design and develop small molecule drugs that target KRAS(G12D) with sufficient bioavailability to treat diseases such as cancer.

### SUMMARY

Provided herein are small molecule inhibitors of the KRAS(G12D) mutant oncoprotein. Inhibitors of KRAS(G12D) include those having the structural formula I:

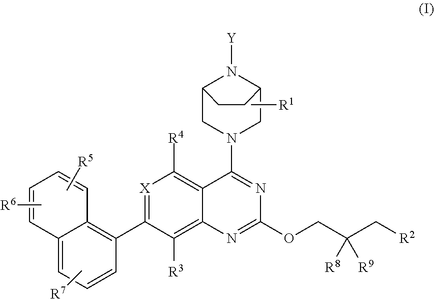

and pharmaceutically acceptable salts and compositions comprising such, wherein Y, X, R¹, R², R³, R⁴, R⁵, R⁶, R⁷, R⁸, and R⁹ are as defined herein. The use of these compounds, salts, and compositions for treating diseases responsive to the inhibition of KRAS(G12D), such as cancer, is also disclosed. In one aspect, disclosed compounds show improved bioavailability. See e.g., Table 3.

### BRIEF DESCRIPTION OF THE FIGURES

FIG. 1 shows the tumor growth inhibition data of female NOD SCID mice treated with Compound 3.

FIG. 2 shows the body weight percent change of female NOD SCID mice treated with Compound 3.

FIG. 3 shows the tumor growth inhibition data of female NOD SCID mice treated with Compound 34.

FIG. 4 shows the body weight percent change of female NOD SCID mice treated with Compound 34.

### DETAILED DESCRIPTION

### 1. General Description of Compounds

As part of a first embodiment, provided is a compound of the Formula I:

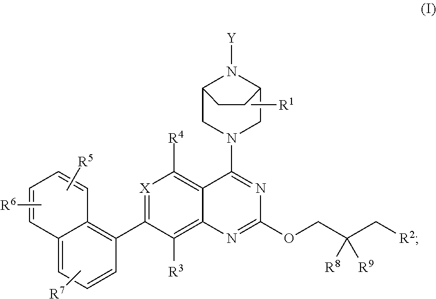

or a pharmaceutically acceptable salt thereof, wherein

Y is hydrogen or —C(O)OCHR^{a}OC(O)R^{b};

X is CH or N;

R¹ is hydrogen, halo, OH, (C₁-C₄)alkyl, (C₁-C₄)haloalkyl, (C₁-C₄)hydroxyalkyl, —CHO, —C(O)OR^{b}, —C(O)ONR^{a}R^{b} or a 5- to 6-membered heteroaryl optionally substituted with 1 to 3 groups selected from halo, (C₁-C₄)alkyl, (C₁-C₄)alkoxy, halo(C₁-C₄)alkoxy, and cyano;

R² is a 4- to 6-membered monocyclic heterocyclyl substituted with 1 to 3 groups selected from R^{c} or a 6- to 10-membered bicyclic heterocyclyl optionally substituted with 1 to 3 groups selected from R^{d};

R³ is selected from hydrogen, halo, (C₁-C₄)alkyl, cyano, and (C₃-C₆)cycloalkyl optionally substituted with 1 to 3 groups selected from halo, (C₁-C₄)alkyl, (C₁-C₄)alkoxy, halo(C₁-C₄)alkoxy, and cyano;

R⁴ is selected from hydrogen, (C₁-C₄)alkyl, (C₁-C₄)haloalkyl, (C₁-C₄)alkoxy, deuterated(C₁-C₄)alkoxy, (C₁-C₄)haloalkoxy, (C₁-C₄)alkynyl, (C₁-C₄)alkenyl, halo, (C₃-C₆)cycloalkyl, —O(C₃-C₆)cycloalkyl, cyano, NH₂, —NH(C₁-C₄)alkyl, —N[(C₁-C₄)alkyl]₂, —P(O)[(C₁-C₄)alkyl]₂, and —S(C₁-C₄)alkyl, wherein said (C₃-C₆)cycloalkyl and said (C₃-C₆)cycloalkyl of —O(C₃-C₆)cycloalkyl are optionally substituted with 1 to 3 groups selected from halo, (C₁-C₄)alkyl, (C₁-C₄)alkoxy, halo(C₁-C₄)alkoxy, and cyano;

R⁵ is (C2-C4)alkynyl;

R⁶ is hydrogen or halo;

R⁷ is hydrogen or OH;

R⁸ and R⁹ are taken together to form ═CH or cyclopropyl;

R^{a} and R^{b} are each independently selected from hydrogen and (C₁-C₄)alkyl; and

R^{c} and R^{d} are each independently selected from halo, (C₁-C₄)alkyl, (C₁-C₄)haloalkyl, (C₁-C₄)alkoxy, (C₁-C₄)haloalkoxy, cyano, OH, oxo, —C(O)OR^{a}, —C(O)R^{a}, —SO₂R^{a}, —S(O)R^{a}, —SO₂NR^{a}R^{b}, —NR^{a}C(O)R^{b}, —NR^{a}SO₂R^{b}, —NR^{a}R^{b}, and NO₂;

provided that R² is not piperazinyl substituted with (C₁-C₄)alkyl, —C(O)OR^{a}, or —C(O)R^{a} when R⁴ is (C₃)alkyl.

### 2. Definitions

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein the term “comprising” or “comprises” are used in reference to compositions, methods, and respective component(s) thereof, that are present in a given embodiment, yet open to the inclusion of unspecified elements.

As used herein, the term “alkyl” means a saturated straight chain or branched non-cyclic hydrocarbon having, unless specified otherwise, from 1 to 10 carbon atom e.g., (C₁-C₆)alkyl or (C₁-C₄)alkyl. Representative straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

As used herein, the term “alkynyl” means a saturated straight chain or branched non-cyclic hydrocarbon having, unless specified otherwise, from 2 to 10 carbon atoms (e.g., (C₂-C₆)alkynyl or (C₂-C₄)alkynyl) and having at least one carbon-carbon triple bond.

Representative straight chain and branched alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like.

As used herein, the term “cycloalkyl” means a saturated, monocyclic alkyl radical having from e.g., 3 to 10 carbon atoms (e.g., from 3 to 6 carbon atoms). Representative cycloalkyls include cyclopropyl, cyclobutyl, cyclopentyl, cyclohexyl, cycloheptyl, cyclooctyl, cyclononyl, and cyclodecanyl.

The term “oxo” refers to the group ═O.

As used herein, the term “haloalkyl” means and alkyl group in which one or more (including all) the hydrogen radicals are replaced by a halo group, wherein each halo group is independently selected from —F, —Cl, —Br, and —I. Representative haloalkyl groups include trifluoromethyl, bromomethyl, 1,2-dichloroethyl, 4-iodobutyl, 2-fluoropentyl, and the like.

“Alkoxy” means an alkyl radical attached through an oxygen linking atom, represented by —O-alkyl. For example, “(C₁-C₄)alkoxy” includes methoxy, ethoxy, proproxy, and butoxy.

“deuterated alkoxy” refers to an alkoxy group in which one or more hydrogens (e.g., one or two hydrogens) has been replaced by deuterium.

“Haloalkoxy” is a haloalkyl group which is attached to another moiety via an oxygen atom such as, e.g., —OCHF₂ or —OCF₃.

As used herein, the term “halogen” or “halo” means F, Cl, Br or I.

As used herein, the term “heterocyclyl” means a 4- to 12-membered monocyclic or polycyclic (e.g., a bridged, fused, or spiro bicyclic ring) saturated or partially unsaturated heterocyclic ring containing 1 to 4 heteroatoms independently selected from N, O, and S. The heterocycle may be attached via any heteroatom or carbon atom, as valency permits. Representative heterocycles include morpholinyl, thiomorpholinyl, pyrrolidinonyl, pyrrolidinyl, piperidinyl, piperazinyl, oxiranyl, dioxanyl, oxetanyl, dihydrofuranyl, dihydropyranyl, isoindolinyl, dihydropyridinyl, tetrahydrofuranyl, tetrahydropyranyl, tetrahydropyrindinyl, tetrahydropyrimidinyl, diazabicyclooctanyl, hexahydropyrrolizinyl, 2-azaspiro[3.3]heptanyl, 2,7-diazaspiro[3.5]nonanyl, 2-azaspiro[3.5]nonanyl, 3-azabicyclo[3.1.0]hexanyl, 2-azabicyclo[3.1.0]hexanyl, 8-azabicyclo[3.2.1]octanyl, 3,8-diazabicyclo[3.2.1]octanyl, 3,6-diazabicyclo[3.1.1]heptanyl, octahydro-1H-pyrrolo[2,3-c]pyridinyl, 1,4-dioxa-8-azaspiro[4.5]decanyl, or 1,2,3,6-tetrahydropyridinyl and the like. Optional substituents on a heterocyclyl group may be present on any substitutable position and, include, e.g., the position at which the heterocyclyl is attached, valence permitting.

The term “spiro” refers to two rings that shares one ring atom (e.g., carbon).

The term “fused” refers to two rings that share two adjacent ring atoms with one another.

The term “bridged” refers to two rings that share three ring atoms with one another.

As used herein, the term “heteroaryl” means a 5- to 12-membered aromatic radical containing 1-4 heteroatoms selected from N, O, and S. A heteroaryl group may be mono- or bicyclic. The heteroaryl may be attached via any heteroatom or carbon atom, as valency permits. Representative heteroaryl groups include pyridyl, furanyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, benzothienyl, and the like. Optional substituents on a heteroaryl group may be present on any substitutable position and, include, e.g., the position at which the heteroaryl is attached, valence permitting.

When used in connection to describe a chemical group that may have multiple points of attachment, a hyphen (-) designates the point of attachment of that group to the variable to which it is defined. For example, —(C₁-C₄)alkylaryl and means that the point of attachment for these groups occurs on the alkyl group.

A hash bond as in “ [symbol] ” represents the point at which the depicted group is attached to the defined variable.

The term “KRAS” refers to the protein product of the KRAS proto-oncogene, GTPase gene.

The term “KRAS(G12D)” refers to the protein product of the KRAS gene carrying a mutation that results in the glycine amino acid at position 12 of KRAS being replaced by an aspartate.

A “chemical entity which binds KRAS^{G12D}” refers to a small molecule or a distinct portion of a larger molecule which binds to a portion of KRAS^{G12D}. In some aspects, the chemical entity which binds KRAS^{G12D} is a small molecule. In some aspects, the chemical entity which binds KRAS^{G12D} is a small molecule having a molecular weight of less than 2,000 g/mol. In some aspects, the chemical entity which binds KRAS^{G12D} induces a confirmation change in KRAS^{G12D}.

The term “SOS1” refers to the protein product of the SOS1 gene that functions as a guanine nucleotide exchange factor for RAS proteins.

The compounds described herein may have chiral centers and/or geometric centers (E- and Z-isomers). It will be understood that the present disclosure encompasses all stereoisomers and geometric isomers. Tautomeric forms of the compounds described herein are also part of the present disclosure.

When the stereochemistry of a disclosed compound is named or depicted by structure, the named or depicted stereoisomer is at least 60%, 70%, 80%, 90%, 99% or 99.9% by weight pure relative to all of the other stereoisomers. Percent by weight pure relative to all of the other stereoisomers is the ratio of the weight of one stereoisomer over the weight of the depicted stereoisomer plus the weight of the other stereoisomers.

For use in medicines, the pharmaceutically acceptable salts of the disclosed compounds refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) of the state of disease, amelioration or palliation of the disease state, diminishing rate of or time to progression, and remission (whether partial or total). “Treatment” can also mean prolonging survival as compared to expected survival in the absence of treatment. Treatment does not need to be curative.

A “therapeutically effective amount” is that amount sufficient to treat a disease in a subject. A therapeutically effective amount can be administered in one or more administrations. In one aspect, a therapeutically effective amount refers to a dosage of from about 0.01 to about 100 mg/kg body weight/day.

The terms “administer,” “administering” or “administration” include any method of delivery of a pharmaceutical composition or agent into a subject's system or to a particular region in or on a subject. In certain embodiments, an agent is administered intravenously, intramuscularly, subcutaneously, intradermally, intranasally, orally, transcutaneously, or mucosally. In certain embodiments, an agent is administered intravenously. In In certain embodiments, an agent is administered orally. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

### 3. Compounds

As part of a second embodiment, the compound of Formula I is of the Formula II:

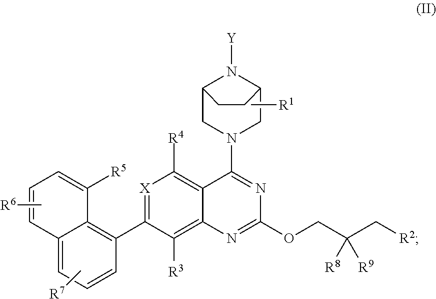

or a pharmaceutically acceptable salt thereof, wherein the variables are as described above for Formula I.

As part of a third embodiment, the compound of Formula I is of the Formula III:

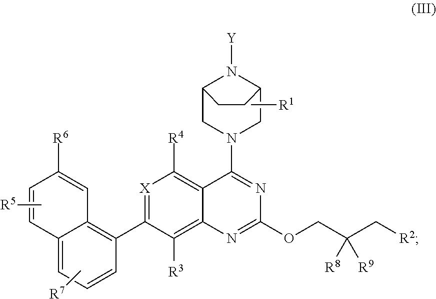

or a pharmaceutically acceptable salt thereof, wherein the variables are as described above for Formula I.

As part of a fourth embodiment, the compound of Formula I is of the Formula IV:

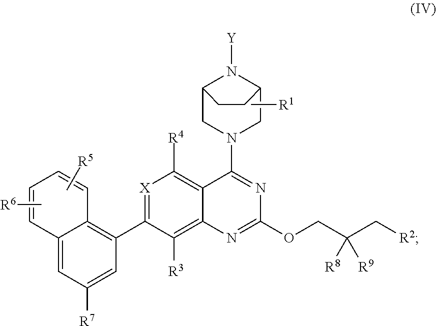

or a pharmaceutically acceptable salt thereof, wherein the variables are as described above for Formula I.

As part of a fifth embodiment, the compound of Formula I is of the Formula V:

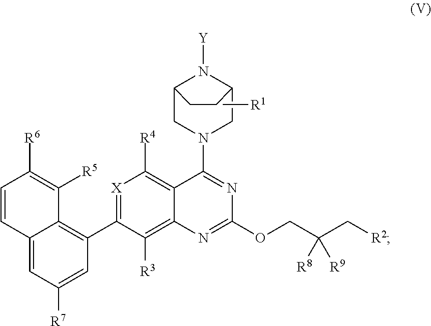

or a pharmaceutically acceptable salt thereof, wherein the variables are as described above for Formula I.

As part of a sixth embodiment, R¹ in the compound of Formula I, II, III, IV, or V, or a pharmaceutically acceptable salt thereof, is hydrogen, wherein the variables are as described above for Formula I.

As part of a seventh embodiment, X in the compound of Formula I, II, III, IV, or V, or a pharmaceutically acceptable salt thereof, is N, wherein the variables are as described above for Formula I or the sixth embodiment.

As part of an eighth embodiment, R³ in the compound of Formula I, II, III, IV, or V, or a pharmaceutically acceptable salt thereof, is halo, wherein the variables are as described above for Formula I or the sixth or seventh embodiment. Alternatively, as part of an eighth embodiment, R³ in the compound of Formula I, II, III, IV, or V, or a pharmaceutically acceptable salt thereof, is fluoro, wherein the variables are as described above for Formula I or the sixth or seventh embodiment.

As part of a ninth embodiment, R⁵ in the compound of Formula I, II, III, IV, or V, or a pharmaceutically acceptable salt thereof, is (C₂)alkynyl, wherein the variables are as described above for Formula I or any one of the sixth to eighth embodiments.

As part of a tenth embodiment, R⁶ in the compound of Formula I, II, III, IV, or V, or a pharmaceutically acceptable salt thereof, is halo, wherein the variables are as described above for Formula I or any one of the sixth to ninth embodiments. Alternatively, as part of a tenth embodiment, R⁶ in the compound of Formula I, II, III, IV, or V, or a pharmaceutically acceptable salt thereof, is fluoro, wherein the variables are as described above for Formula I or any one of the sixth to ninth embodiments.

As part of an eleventh embodiment, R⁷ in the compound of Formula I, II, III, IV, or V, or a pharmaceutically acceptable salt thereof, is OH, wherein the variables are as described above for Formula I or any one of the sixth to tenth embodiments.

As part of a twelfth embodiment, R⁴ in the compound of Formula I, II, III, IV, or V, or a pharmaceutically acceptable salt thereof, is selected from hydrogen, (C₁-C₄)alkoxy, deuterated(C₁-C₄)alkoxy, —N[(C₁-C₄)alkyl]₂, halo, (C₃-C₆)cycloalkyl, (C₁-C₄)haloalkoxy, (C₁-C₄)alkyl, and NH₂, wherein the variables are as described above for Formula I or any one of the sixth to eleventh embodiments. Alternatively, as part of a twelfth embodiment, R⁴ in the compound of Formula I, II, III, IV, or V, or a pharmaceutically acceptable salt thereof, is selected from hydrogen, methyl, methoxy, isopropoxy, OCDF₂, —OCHF₂, —N(CH₃)₂, NH₂, chloro, and cyclopropyl, wherein the variables are as described above for Formula I or any one of the sixth to eleventh embodiments.

As part of a thirteenth embodiment, R² in the compound of Formula I, II, III, IV, or V, or a pharmaceutically acceptable salt thereof, is a 4- to 6-membered nitrogen containing monocyclic heterocyclyl substituted with 1 to 3 groups selected from R^{c} or a 7- to 10-membered nitrogen containing fused or spiro bicyclic heterocyclyl optionally substituted with 1 to 3 groups selected from R^{d}, wherein the variables are as described above for

Formula I or any one of the sixth to twelfth embodiments. Alternatively, as part of a thirteenth embodiment, R² in the compound of Formula I, II, III, IV, or V, or a pharmaceutically acceptable salt thereof, is azetidinyl, piperidinyl, morpholinyl, or pyrrolidinyl, each of which being substituted with 1 to 3 groups selected from R^{c} or R² in the compound of Formula I, II, III, IV, or V, or a pharmaceutically acceptable salt thereof, 3-azabicyclo[3.1.0]hexanyl, 2-azabicyclo[3.1.0]hexanyl, 1,4-dioxa-8-azaspiro[4.5]decanyl, or 1,2,3,6-tetrahydropyridinyl, each of which being optionally substituted with 1 to 3 groups selected from R^{d}, wherein the variables are as described above for Formula I or any one of the sixth to twelfth embodiments.

As part of a fourteenth embodiment, R^{c} and R^{d} in the compound of Formula I, II, III, IV, or V, or a pharmaceutically acceptable salt thereof, are each independently selected from halo, cyano, (C₁-C₄)haloalkyl, (C₁-C₄)alkoxy, (C₁-C₄)alkyl, (C₁-C₄)haloalkoxy, —S(O)R^{a}, and —SO₂NR^{a}R^{b}, wherein the variables are as described above for Formula I or any one of the sixth to thirteenth embodiments. Alternatively, as part of a fourteenth embodiment, R^{c} and R^{d} in the compound of Formula I, II, III, IV, or V, or a pharmaceutically acceptable salt thereof, are each independently selected from fluoro, cyano, CF₃, methoxy, isopropyl, OCF₃, —S(O)CH₃, and —SO₂N(CH₃)₂, wherein the variables are as described above for Formula I or any one of the sixth to thirteenth embodiments.

As part of a fifteenth embodiment, R⁸ and R⁹ in the compound of Formula I, II, III, IV, or V, or a pharmaceutically acceptable salt thereof, are taken together to form cyclopropyl, wherein the variables are as described above for Formula I or any one of the sixth to fourteenth embodiments.

As part of a sixteenth embodiment, Y in the compound of Formula I, II, III, IV, or V, or a pharmaceutically acceptable salt thereof, is hydrogen or —C(O)OCH(CH₃)OC(O)CH₃, wherein the variables are as described above for Formula I or any one of the sixth to fifteenth embodiments. Alternatively, as part of a sixteenth embodiment, Y in the compound of Formula I, II, III, IV, or V, or a pharmaceutically acceptable salt thereof, is hydrogen, wherein the variables are as described above for Formula I or any one of the sixth to fifteenth embodiments.

Additional compounds are further disclosed in the Exemplification and are included in the present disclosure. Pharmaceutically acceptable salts thereof as well as the neutral forms are included.

### 4. Uses, Formulation and Administration

Compounds and compositions described herein are generally useful as anticancer therapies. In one aspect, the disclosed compounds and compositions behave as inhibitors of KRAS(G12D). Their mechanisms of action include, but are not limited to, inhibiting KRAS(G12D) and thereby impeding down-stream signals that may result in inhibition of cancer cell growth and/or induction of cancer cell death or other KRAS or KRAS(G12D) functions. In one aspect, the disclosed compounds effectuate the inhibition of KRAS(G12D).

Thus, provided herein are methods of treating conditions which are responsive to the inhibition of KRAS(G12D) comprising administering to a subject in need thereof, a therapeutically effective amount of one or more compounds or compositions described herein. Also provided is the use of one or more compounds or compositions described herein in the manufacture of a medicament for treating conditions which are responsive to the inhibition of KRAS(G12D). Further provided is the use of a compound or composition described herein for treating conditions which are responsive to the inhibition of KRAS(G12D).

In one aspect, the condition treated by the present compounds and compositions is a cancer. The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. Cancer cells are often in the form of a solid tumor. However, cancer also includes non-solid tumors, e.g., blood tumors, e.g., leukemia, wherein the cancer cells are derived from bone marrow. As used herein, the term “cancer” includes pre-malignant as well as malignant cancers. Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin and non-Hodgkin), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

“Solid tumor,” as used herein, is understood as any pathogenic tumor that can be palpated or detected using imaging methods as an abnormal growth having three dimensions. A solid tumor is differentiated from a blood tumor such as leukemia. However, cells of a blood tumor are derived from bone marrow; therefore, the tissue producing the cancer cells is a solid tissue that can be hypoxic.

“Tumor tissue” or “tumorous tissue” are understood as cells, extracellular matrix, and other naturally occurring components associated with the solid tumor.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

### EXEMPLIFICATION

### Chemical Synthesis

The representative examples that follow are intended to help illustrate the present disclosure, and are not intended to, nor should they be construed to, limit the scope of the invention. General starting materials used were obtained from commercial sources or prepared in other examples, unless otherwise noted.

The compounds claimed herein were prepared following the procedures outlined in the Scheme 1.

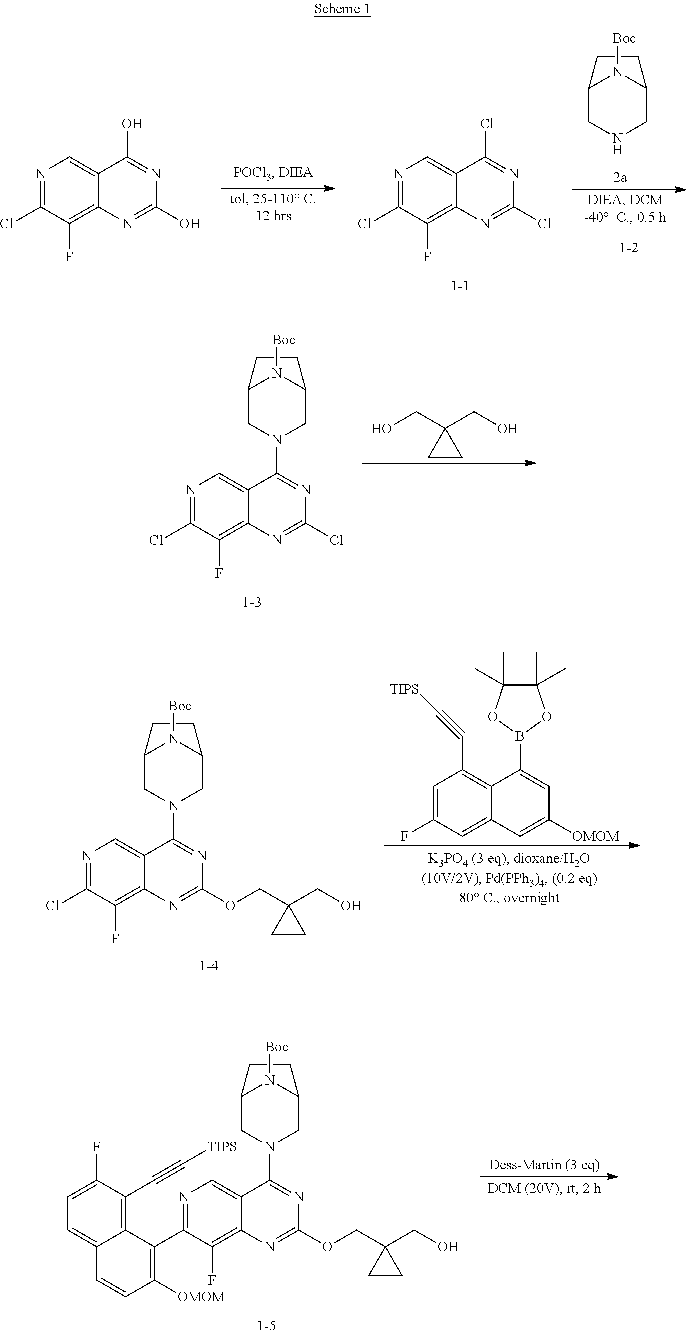

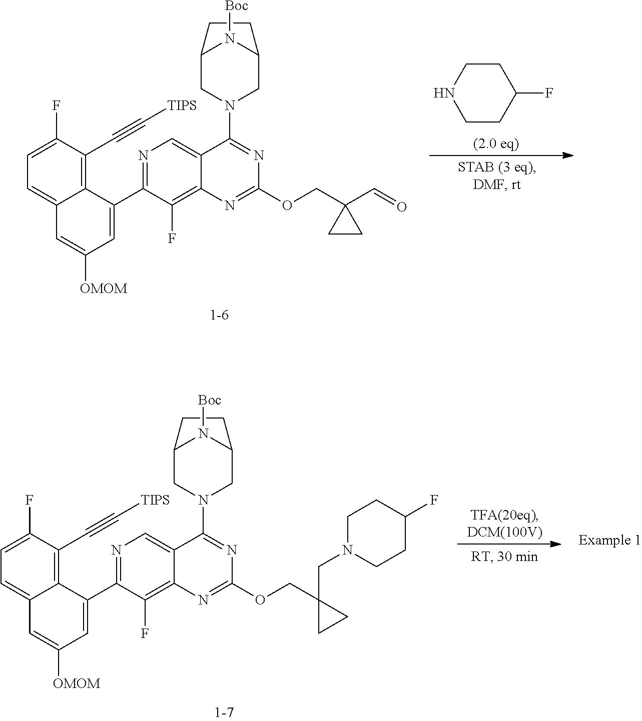

### Preparation of Example 1

Step 1: 2,4,7-trichloro-8-fluoropyrido[4,3-d]pyrimidine. To a mixture of Intermediate 1-1 (50.0 g, 232 mmol, 1.00 eq) in Tol. (150 mL) was added POCl₃ (178 g, 1.16 mol, 108 mL, 5.00 eq) at 25° C. Then DIEA (65.9 g, 510 mmol, 88.9 mL, 2.20 eq) was added into the mixture blow 40° C. The mixture was stirred at 110° C. for 12 hrs. LC-MS showed desired MS was detected. The reaction mixture was distilled under reduced pressure to remove POCl₃ at 90° C. The residue was poured into Sat. NaHCO₃ slowly (keep pH=8). During this period, yellow precipitate was formed. It was collected by filtration and washed by H₂O. The solid was used for next step directly. Intermediate 1-2 (101 g, 400 mmol, 86.2% yield) was obtained as brown solid. HNMR (DMSO-d₆, 400 MHz): δ 8.92-8.86 (m, 1H). LC-MS: m/z 253.9 [M+H]⁺.

Step 2: tert-butyl 3-(2,7-dichloro-8-fluoropyrido[4,3-d]pyrimidin-4-yl)-3,8-diazabicyclo[3.2.1]octane-8-carboxylate. To a mixture of Intermediate 1-2 (48.5 g, 192 mmol, 1.00 eq) in DCM (485 mL) was added a solution of compound 2a (38.7 g, 183 mmol, 0.950 eq) in DCM (120 mL). Then DIEA (49.7 g, 384 mmol, 66.9 mL, 2.00 eq) was added into the mixture at −40° C. and stirred at −40° C.for 0.5 hr under N₂. LCMS showed intermediate 1-2 was consumed, and desired MS was detected. The mixture was quenched by HCl (0.5 M) and the pH of the aqueous phase was acidified to 6˜7, then separated, the organic layer was dried over Na₂SO₄. The residue was purified by column chromatography (SiO₂, TLC: Petroleum ether:Ethyl acetate=3:1, R_{f}=0.4, Petroleum ether:Ethyl acetate=10:1 to 1:1, R_{f}=0.4). Intermediate 1-3 (120 g, 280 mmol, 72.9% yield) was obtained as white solid. HNMR (DMSO-d₆, 400 MHz): δ 9.13-8.98 (m, 1H), 4.67-4.36 (m, 2H), 4.35-4.21 (m, 2H), 3.87-3.50 (m, 2H), 1.85-1.71 (m, 2H), 1.66-1.56 (m, 2H), 1.46 (s, 9H). LC-MS: m/z 428.0[M+H]⁺.

Step 3: tert-butyl 3-(7-chloro-8-fluoro-2-((1-(hydroxymethyl)cyclopropyl)methoxy)pyrido[4,3-d]pyrimidin-4-yl)-3,8-diazabicyclo[3.2.1]octane-8- carboxylate. To a stirred mixture of [1-(hydroxymethyl)cyclopropyl]methanol (14.31 g, 140 mmol, 3 eq) and t-BuONa (13.46 g, 140 mmol, 3 eq) in THF was added tert-butyl 3-{2,7-dichloro-8-fluoropyrido[4,3-d]pyrimidin-4-yl}-3,8-diazabicyclo[3.2.1]octane-8-carboxylate (20 g, 46.697 mmol, 1 eq) in portions at 0° C. under nitrogen atmosphere. The resulting mixture was stirred for 1 h under nitrogen atmosphere. The reaction was quenched with Water at 0° C. The resulting mixture was extracted with CH₂Cl₂ (3×10mL). The combined organic layers were washed with sat. NaCl aq. (2×5 mL), dried over anhydrous Na₂SO₄. The resulting mixture was concentrated under reduced pressure. The residue was purified by silica gel column chromatography, eluted with PE/EA (1:1) to afford intermediate 1-4 (14.5 g, 62.86%) as a white solid. LCMS (ES, m/z): 494 [M+H]+

Step 4: tert-butyl 3-(8-fluoro-7-(7-fluoro-2-(methoxymethoxy)-8-((triisopropylsilyl)ethynyl)naphthalen-1-yl)-2-((1-(hydroxymethyl)cyclopropyl)methoxy)pyrido[4,3-d]pyrimidin-4-yl)-3,8-diazabicyclo[3.2.1]octane-8-carboxylate. A solution of intermediate 1-4 (3 g, 6.073 mmol, 1.0 eq) and ((3-fluoro-6-(methoxymethoxy)-8-(4,4,5,5-tetramethyl-1,3,2-dioxaborolan-2-yl)naphthalen-1- yl)ethynyl)triisopropylsilane (2.63 g, 9.110 mmol, 1.5 eq) in 1,4-dioxane (40 mL) was treated with Pd(PPh₃)₄ (1.40 g, 1.215 mmol, 0.2 eq) and K₃P O₄ (3.87 g, 18.219 mmol, 3.0 eq) under nitrogen atmosphere. The resulting mixture was stir ed for overnight at 80° C. under nitrogen atmosphere. The reaction was quenched with Water at room temperature. The resulting mixture was extracted with CH₂Cl₂ (3×10 mL). The co mbined organic layers were washed with sat. NaCl aq. (2×5 mL), dried over anhydrous Na₂ SO₄. The resulting mixture was concentrated under reduced pressure. The residue was purifi ed by silica gel column chromatography, eluted with CH₂Cl₂/MeOH (10:1) to afford intermediate 1-5 (2.5 g, 66.38%) as a yellow solid. LCMS (ES, m/z): 620 [M+H]⁺

Step 5: tert-butyl 3-(8-fluoro-7-(7-fluoro-3-(methoxymethoxy)-8-((triisopropylsilyl)ethynyl)naphthalen-1-yl)-2-((1-formylcyclopropyl)methoxy)pyrido[4,3- d]pyrimidin-4-yl)-3,8-diazabicyclo[3.2.1]octane-8-carboxylate. To a stirred solution of intermediate 5 (200 mg, 0.323 mmol, 1 eq) in DCM was added Dess-Martin (410.38 mg, 0.969 mmol, 3 eq) in portions at room temperature. The resulting mixture was stirred for 2 h at room temperature. The reaction was quenched with sat. NaHCO₃ (aq.) at 0° C. The resulting mixture was extracted with CH2Cl2 (3×10 mL). The combined organic layers were washed with sat. NaCl (aq.) (3×5 mL), dried over anhydrous Na₂SO₄. After filtration, the filtrate was concentrated under reduced pressure. The crude product was used in the next step directly without further purification. LCMS (ES, m/z): 618 [M+H]⁺

Step 6: tert-butyl 3-(8-fluoro-7-(7-fluoro-3-(methoxymethoxy)-8-((triisopropylsilyl)ethynyl)naphthalen-1-yl)-2-((1-((4-fluoropiperidin-1- yl)methyl)cyclopropyl)methoxy)pyrido[4,3-d]pyrimidin-4-yl)-3,8-diazabicyclo[3.2.1]octane-8-carboxylate. To a stirred solution of intermediate 1-6 (100 mg, 0.162 mmol, 1 eq) and 4-fluoropiperidine hydrochloride (45.17 mg, 0.324 mmol, 2 eq) in DMF were added STAB (102.87 mg, 0.486 mmol, 3 eq) in portions at room temperature. The resulting mixture was stirred for 16 h at room temper ature. The reaction was quenched with Water at room temperature. The resulting mixture w as extracted with EtOAc (3×10 mL). The combined organic layers were washed with sat. N aCl aq. (2×5 mL), dried over anhydrous Na₂SO₄. After filtration, the filtrate is concentrated under reduced pressure. The crude product was used in the next step directly without further purification. LCMS (ES, m/z): 605 [M+H]⁺

Step 7: 4-(4-(3,8-diazabicyclo[3.2.1]octan-3-yl)-8-fluoro-2-((1-((4-fluoropiperidin-1-yl)methyl)cyclopropyl)methoxy)pyrido[4,3-d]pyrimidin-7-yl)-5-ethynyl-6-fluoronaphthalen-2-ol. To a stirred solution of intermediate 1-7 (80 mg, 0.132 mmol, 1.0 eq) in DCM was added TFA (1 mL, 13.463 mmol, 101.84 eq) dropwise at room temperature. The resulting mixture was stirred for 1 h at room temperature. The resulting mixture was concentrated under reduced pressure. The crude product was purified by Prep-HPLC with the following conditions (Column, XBridge Prep OBD C₁₈ Column, 30×150 mm, 5 μm; mobile phase, Water (10 mmol/L NH4HCO3) and ACN (30% ACN up to 80% in 10 min); Detector, uv 220 nm) to afford the compound of example 1 (38.15 mg, 47.69%) as a white solid. ¹H NMR (DMSO-d₆, 400 MHz): δ 10.15 (s, 1H), 9.03 (s, 1H), 7.97 (dd, J=9.2, 6.0 Hz, 1H), 7.46 (t, J=9.2 Hz, 1H), 7.39 (d, J=2.4 Hz, 1H), 7.17 (d, J=2.4 Hz, 1H), 4.69 (dt, J=7.6, 3.6 Hz, 1H), 4.48 (d, J=12.0 Hz, 1H), 4.34-4.22 (m, 3H), 3.93 (d, J=1.2 Hz, 1H), 3.67-3.59 (m, 1H), 3.54 (t, J=5.6 Hz, 3H), 2.74 (s, 1H), 2.56 (s, 2H), 2.30 (t, J=7.6 Hz, 4H), 1.88-1.76 (m, 2H), 1.66 (s, 6H), 0.64 (q, J=3.2 Hz, 2H), 0.40 (t, J=3.2 Hz, 2H). LC-MS: m/z 628.9 [M+H]⁺.

The following compounds in Table 1 were prepared according to the methods described above using the appropriate starting materials.

TABLE 1

| | | LCMS: | |
|---|---|---|---|
| | | m/z | ¹H NMR |
| Cmp | Structure | [M + H]⁺ | (DMSO-d₆, 400 MHz) |
| Ref. 1 | 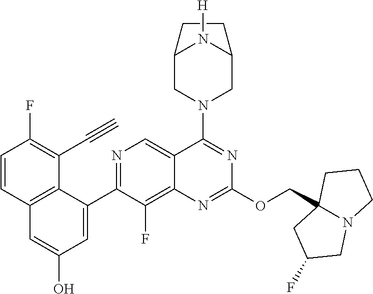 | 601.3 | |
| Ref. 2 | 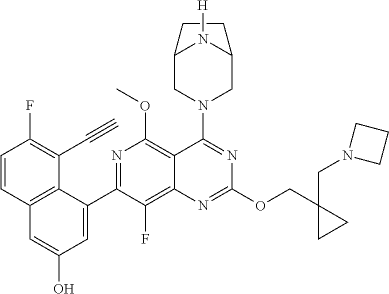 [SMILES: C#Cc1c(F)ccc2cc(O)cc(-c3nc(OC)c4c(N5CC6CCC(C5)N6)nc(OCC5(CN6CCC6)CC5)nc4c3F)c12] | 612.8 | 10.14 (s, 1H), 7.97 (dd, J = 9.2, 5.9 Hz, 1H), 7.46 (t, J = 9.0 Hz, 1H), 7.38 (d, J = 2.6 Hz, 1H), 7.22 (d, J = 2.6 Hz, 1H), 4.18 (s, 2H), 4.10 (d, J = 12.2 Hz, 1H), 3.89 (d, J = 5.4 Hz, 4H), 3.50 (s, 2H), 3.40 (s, 2H), 3.13 (t, J = 6.9 Hz, 4H), 2.38 (s, 2H), 1.94 (p, J = 6.8 Hz, 2H), 1.62 (d, J = 13.9 Hz, 4H), 0.50 (d, J = 4.2 Hz, 2H), 0.43 (d, J = 4.1 Hz, 2H). |
| Ref. 3 | 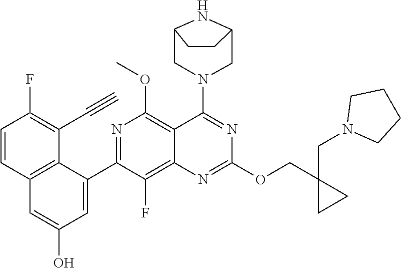 [SMILES: C#Cc1c(F)ccc2cc(O)cc(-c3nc(OC)c4c(N5CC6CCC(C5)N6)nc(OCC5(CN6CCCC6)CC5)nc4c3F)c12] | 627.0 | δ 10.14 (s, 1H), 7.97 (dd, J = 9.2, 5.9 Hz, 1H), 7.46 (t, J = 9.0 Hz, 1H), 7.38 (d, J = 2.6 Hz, 1H), 7.21 (d, J = 2.6 Hz, 1H), 4.24 (s, 2H), 4.09 (s, 1H), 3.88 (d, J = 6.4 Hz, 5H), 3.46 (s, 2H), 3.39 (s, 2H), 2.47 − 2.40 (m, 4H), 2.39 − 2.31 (m, 2H), 1.69 − 1.52 (m, 8H), 0.60 (q, J = 4.0 Hz, 2H), 0.42 (q, J = 4.2 Hz, 2H) |
| Ref. 4 | 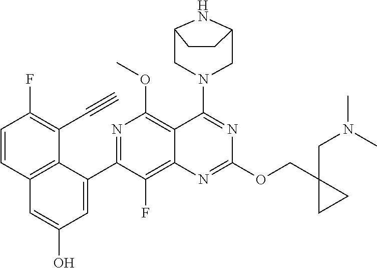 [SMILES: C#Cc1c(F)ccc2cc(O)cc(-c3nc(OC)c4c(N5CC6CCC(C5)N6)nc(OCC5(CN(C)C)CC5)nc4c3F)c12] | 600.7 | δ 8.78 (s, 1H), 8.21 (s, 1H), 7.97 (dd, J = 9.2, 5.9 Hz, 1H), 7.46 (t, J = 9.0 Hz, 1H), 7.38 (d, J = 2.6 Hz, 1H), 7.22 (d, J = 2.6 Hz, 1H), 4.22 (d, J = 1.5 Hz, 2H), 4.11 (d, J = 12.5 Hz, 1H), 3.92 (d, J = 1.1 Hz, 5H), 3.60 (s, 2H), 3.40 (dd, J = 18.6, 12.7 Hz, 2H), 2.31 (m, 1H), 2.17 (s, 6H), 1.66 (d, J = 13.3 Hz, 4H), 0.63 (q, J = 4.0 Hz, 2H), 0.37 (m, 2H). |
| 1 | 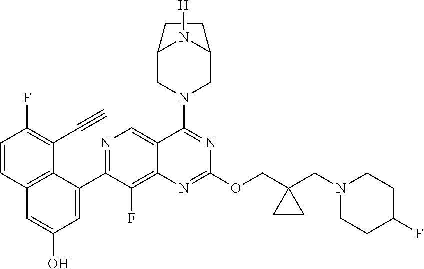 [SMILES: C#Cc1c(F)ccc2cc(O)cc(-c3ncc4c(N5CC6CCC(C5)N6)nc(OCC5(CN6CCC(F)CC6)CC5)nc4c3F)c12] | 628.9 | 10.15 (s, 1H), 9.03 (s, 1H), 7.97 (dd, J = 9.2, 6.0 Hz, 1H), 7.46 (t, J = 9.2 Hz, 1H), 7.39 (d, J = 2.4 Hz, 1H), 7.17 (d, J = 2.4 Hz, 1H), 4.69 (dt, J = 7.6, 3.6 Hz, 1H), 4.48 (d, J = 12.0 Hz, 1H), 4.34 − 4.22 (m, 3H), 3.93 (d, J = 1.2 Hz, 1H), 3.67 − 3.59 (m, 1H), 3.54 (t, J = 5.6 Hz, 3H), 2.74 (s, 1H), 2.56 (s, 2H), 2.30 (t, J = 7.6 Hz, 4H), 1.88 − 1.76 (m, 2H), 1.66 (s, 6H), 0.64 (q, J = 3.2 Hz, 2H), 0.40 (t, J = 3.2 Hz, 2H). |
| 2 | 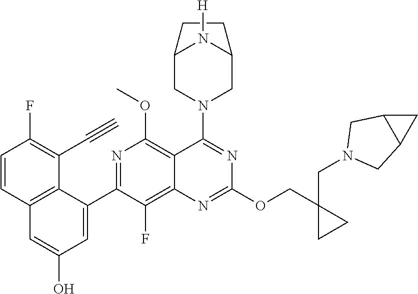 [SMILES: C#Cc1c(F)ccc2cc(O)cc(-c3nc(OC)c4c(N5CC6CCC(C5)N6)nc(OCC5(CN6CC7CC7C6)CC5)nc4c3F)c12] | 639.9 | 10.13 (s, 1H), 7.97 (dd, J = 9.2, 5.9 Hz, 1H), 7.46 (t, J = 9.2 Hz, 1H), 7.38 (d, J = 2.4 Hz, 1H), 7.21 (d, J = 2.4 Hz, 1H), 4.26 − 4.15 (m, 2H), 4.11 (d, J = 12.0 Hz, 1H), 3.87 (d, J = 5.2 Hz, 4H), 3.35 (d, J = 8.0 Hz, 1H), 3.30 − 3.27 (m, 1H), 2.96 (dd, J = 8.4, 5.2 Hz, 2H), 2.43 − 2.23 (m, 2H), 1.60 − 1.53 (m, 4H), 1.32 (dd, J = 7.2, 3.6 Hz, 2H), 0.56 (q, J = 4.0, 2.8 Hz, 3H), 0.41 − 0.34 (m, 2H), 0.25 (td, J = 7.6, 3.6 Hz, 1H). |
| 3 | 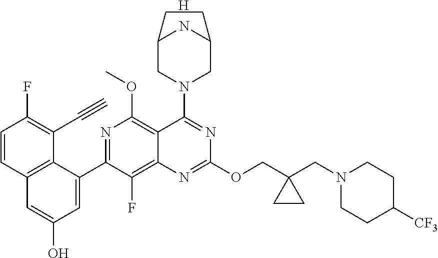 | 709.5 | (MeOH-d₆, 400 MHz): δ 7.89- 7.76 (m, 1H), 7.36-7.28 (m, 2H), 7.24 (d, J = 2.4 Hz, 1H), 4.49- 4.30 (m, 3H), 4.16-4.06 (m, 1H), 4.01 (s, 3H), 3.64-3.55 (m, 3H), 3.47 (d, J = 13.0 Hz, 1H), 3.16 (t, J = 12.2 Hz, 2H), 2.52-2.46 (m, 1H), 2.41-2.34 (m, 1H), 2.15-2.05 (m, 1H), 1.99-1.71 (m, 8H), 1.70- 1.63 (m, 1H), 1.61-1.48 (m, 2H), 0.77-0.67 (m, 2H), 0.54-0.45 (m, 2H). |
| 4 | 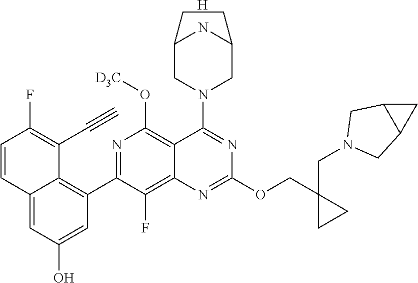 | 642.2 | 8.09-7.94 (m, 1H), 7.77-7.68 (m, 1H), 7.22 (t, J = 8.9 Hz, 1H), 7.14 (d, J = 2.0 Hz, 1H), 6.97 (s, 1H), 4.00-3.92 (m, 2H), 3.92-3.82 (m, 1H), 3.66-3.61 (m, 2H), 3.27 (s, 4H), 2.72 (dd, J = 5.4, 8.4 Hz, 2H), 2.16-2.08 (m, 2H), 1.99 (d, J = 8.4 Hz, 2H), 1.47-1.28 (m, 4H), 1.11-1.04 (m, 2H), 0.37-0.29 (m, 3H), 0.14 (s, 2H), 0.05-0.02 (m, 1H). |
| 5 | 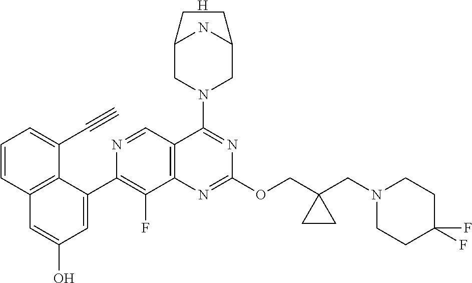 | 629.1 | 10.12 (s, 1H), 9.02 (s, 1H), 7.88 (d, J = 8.0 Hz, 1H), 7.48 − 7.39 (m, 2H), 7.33 (d, J = 2.8 Hz, 1H), 7.10 (d, J = 2.8 Hz, 1H), 4.51 − 4.48 (m, 1H), 4.28 (d, J = 7.6 Hz, 3H), 3.66-3.63 (m, 1H), 3.56 (d, J = 3.6 Hz, 4H), 2.67 (p, J = 2.0 Hz, 3H), 2.40 − 2.31 (m, 2H), 1.96 − 1.83 (m, 5H), 1.66 (s, 4H), 1.24 (s, 1H), 0.65 (d, J = 4.4 Hz, 2H), 0.43 (s, 2H). |
| 6 | 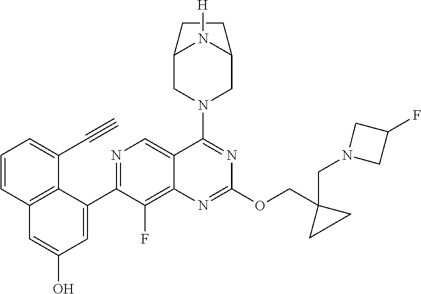 [SMILES: C#Cc1cccc2cc(O)cc(-c3ncc4c(N5CC6CCC(C5)N6)nc(OCC5(CN6CC(F)C6)CC5)nc4c3F)c12] | 583.1 | 10.11 (s, 1H), 9.02 (s, 1H), 7.88 (dd, J = 8.0, 1.7 Hz, 1H), 7.49 − 7.37 (m, 2H), 7.33 (d, J = 2.8 Hz, 1H), 7.11 (d, J = 2.8 Hz, 1H), 5.28 − 4.98 (m, 1H), 4.47 (d, J = 12.0 Hz, 1H), 4.28 (d, J = 12.4 Hz, 1H), 4.18 (s, 2H), 3.61 (s, 2H), 3.58 (s, 2H), 3.56 − 3.50 (m, 4H), 3.11 (dd, J = 8.4, 4.7 Hz, 1H), 3.05 (dd, J = 8.8, 4.8 Hz, 1H),2.48 (s, 2H), 1.66 (s, 4H), 0.56 − 0.48 (m, 2H), 0.46 (d, J = 4.4 Hz, 2H). |
| 7 | 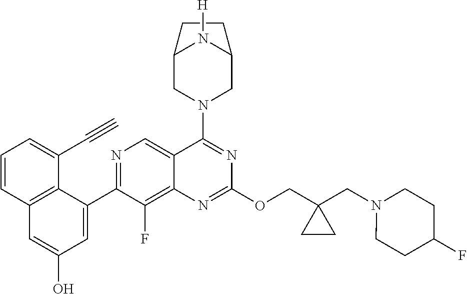 [SMILES: C#Cc1cccc2cc(O)cc(-c3ncc4c(N5CC6CCC(C5)N6)nc(OCC5(CN6CCC(F)CC6)CC5)nc4c3F)c12] | 611.1 | 10.14 (s, 1H), 9.02 (s, 1H), 7.88 (dd, J = 8.0, 1.7 Hz, 1H), 7.51 − 7.38 (m, 2H), 7.34 (d, J = 2.4 Hz, 1H), 7.11 (d, J = 2.4 Hz, 1H), 4.74 − 4.54 (m, 1H), 4.52 − 4.48 (m, 1H), 4.34 − 4.19 (m, 3H), 3.65 (d, J = 12.0 Hz, 1H), 3.57 (d, J = 6.8 Hz, 4H), 3.33 (s, 2H), 2.55 (s, 2H), 2.32 (dd, J = 7.0, 4.0 Hz, 3H), 1.85 − 1.79 (m, 2H), 1.67 (s, 5H), 0.64 (q, J = 4.0, 3.5 Hz, 2H), 0.47 − 0.36 (m, 2H). |
| 8 | 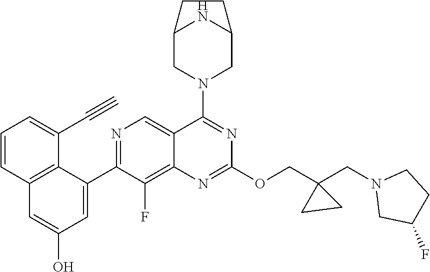 [SMILES: C#Cc1cccc2cc(O)cc(-c3ncc4c(N5CC6CCC(C5)N6)nc(OCC5(CN6CC[C@H](F)C6)CC5)nc4c3F)c12] | 597.1 | 10.14 (s, 1H), 9.02 (s, 1H), 7.88 (dd, J = 8.0, 1.7 Hz, 1H), 7.49 − 7.39 (m, 2H), 7.33 (d, J = 2.8 Hz, 1H), 7.11 (d, J = 2.8 Hz, 1H), 5.31 − 4.96 (m, 1H), 4.50 (d, J = 12.0 Hz, 1H), 4.28 (dd, J = 10.8, 2.9 Hz, 2H), 4.20 − 4.15 (m, 1H), 3.71 − 3.46 (m, 5H), 2.84 (m, 2H), 2.71 − 2.51 (m, 1H), 2.47 (s, 1H), 2.46 − 2.36 (m, 2H), 2.34 − 2.25 (m, 1H), 2.19 − 2.10 (m, 1H), 1.84 − 1.78 (m, 1H), 1.67 (s, 4H), 0.62 (q, J = 3.6 Hz, 2H), 0.48 − 0.38 (m, 2H). |
| 9 | 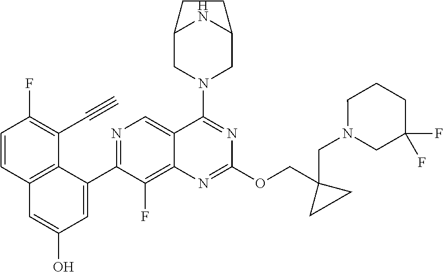 [SMILES: C#Cc1c(F)ccc2cc(O)cc(-c3ncc4c(N5CC6CCC(C5)N6)nc(OCC5(CN6CCCC(F)(F)C6)CC5)nc4c3F)c12] | 647.0 | 10.14 (s, 1H), 9.02 (s, 1H), 7.97 (dd, J = 9.4, 6.0 Hz, 1H), 7.46 (t, J = 9.0 Hz, 1H), 7.39 (d, J = 2.4 Hz, 1H), 7.17 (d, J = 2.4 Hz, 1H), 4.48 (d, J = 12.0 Hz, 1H), 4.33 − 4.18 (m, 3H), 3.92 (s, 1H), 3.64 (d, J = 12.0 Hz, 1H), 3.53 (d, J = 8.0 Hz, 3H), 2.72 − 2.62 (m, 2H), 2.45 (s, 2H), 2.40 − 2.31 (m, 2H), 1.91 − 1.78 (m, 2H), 1.69 − 1.59 (m, 6H), 0.71 − 0.63 (m, 2H), 0.46 − 0.39 (m, 2H). |
| 10 | 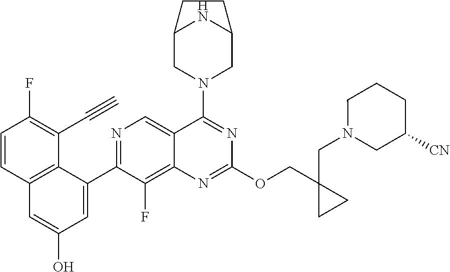 [SMILES: C#Cc1c(F)ccc2cc(O)cc(-c3ncc4c(N5CC6CCC(C5)N6)nc(OCC5(CN6CCC[C@H](C#N)C6)CC5)nc4c3F)c12] | 636.0 | 10.14 (s, 1H), 9.02 (d, J = 1.7 Hz, 1H), 7.97 (dd, J = 9.2, 6.0 Hz, 1H), 7.46 (t, J = 9.2 Hz, 1H), 7.39 (d, J = 2.4 Hz, 1H), 7.17 (d, J = 2.4 Hz, 1H), 4.49 (d, J = 12.8 Hz, 1H), 4.37 (t, J = 11.2 Hz, 1H), 4.29 (d, J = 12.4 Hz, 1H), 4.19 (dd, J = 14.4, 10.8 Hz, 1H), 3.91 (d, J = 8.4 Hz, 1H), 3.64 (d, J = 12.4 Hz, 1H), 3.59 − 3.50 (m, 3H), 2.97 (s, 1H), 2.73 − 2.55 (m, 2H), 2.45 − 2.22 (m, 4H), 1.73 − 1.58 (m, 7H), 0.66 (d, J = 5.6 Hz, 2H), 0.42 (s, 2H). |
| 11 | 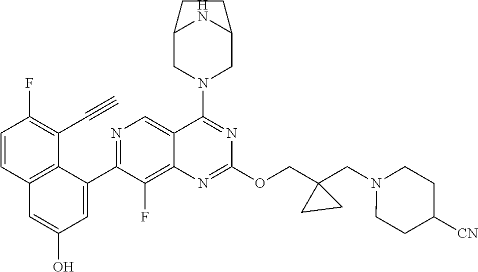 [SMILES: C#Cc1c(F)ccc2cc(O)cc(-c3ncc4c(N5CC6CCC(C5)N6)nc(OCC5(CN6CCC(C#N)CC6)CC5)nc4c3F)c12] | 636.0 | 10.14 (s, 1H), 9.03 (s, 1H), 7.97 (dd, J = 9.2, 6.0 Hz, 1H), 7.46 (t, J = 9.2 Hz, 1H), 7.39 (d, J = 2.4 Hz, 1H), 7.17 (d, J = 2.4 Hz, 1H), 4.52 − 4.46 (m, 1H), 4.26 (d, J = 7.6 Hz, 3H), 3.93 (d, J = 1.2 Hz, 1H), 3.69 − 3.62 (m, 1H), 3.58 − 3.52 (m, 3H), 2.82 (s, 1H), 2.58 (s, 2H), 2.40 − 2.19 (m, 4H), 1.88 − 1.80 (m, 2H), 1.67 (s, 6H), 0.64 (q, J = 3.2 Hz, 2H), 0.41 (t, J = 2.8 Hz, 2H). |
| 12 | 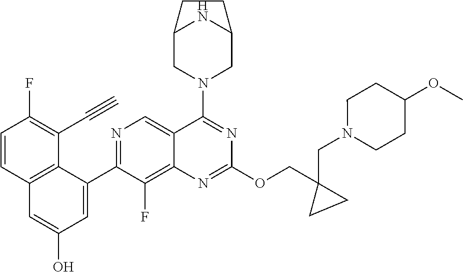 [SMILES: C#Cc1c(F)ccc2cc(O)cc(-c3ncc4c(N5CC6CCC(C5)N6)nc(OCC5(CN6CCC(OC)CC6)CC5)nc4c3F)c12] | 641.0 | 10.14 (s, 1H), 9.02 (s, 1H), 7.97 (dd, J = 9.2, 6.0 Hz, 1H), 7.46 (t, J = 9.2 Hz, 1H), 7.39 (d, J = 2.4 Hz, 1H), 7.17 (d, J = 2.4 Hz, 1H), 4.52 − 4.46 (m, 1H), 4.37 − 4.18 (m, 3H), 3.93 (d, J = 1.2 Hz, 1H), 3.70 − 3.61 (m, 1H), 3.58 − 3.52 (m, 3H), 3.19 (s, 3H), 3.11 (dq, J = 8.4, 4.4 Hz, 1H), 2.81 − 2.63 (m, 2H), 2.36 − 2.20 (m, 2H), 2.04 (s, 2H), 1.82 − 1.78 (m, 2H), 1.67 (s, 4H), 1.43 − 1.29 (m, 2H), 0.63 (q, J = 3.6 Hz, 2H), 0.40 (t, J = 3.2 Hz, 2H). |
| 13 | 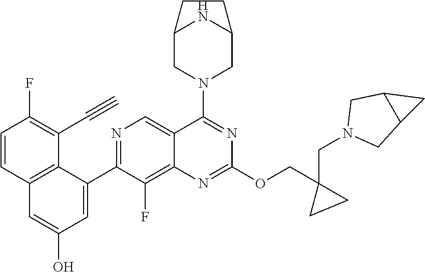 [SMILES: C#Cc1c(F)ccc2cc(O)cc(-c3ncc4c(N5CC6CCC(C5)N6)nc(OCC5(CN6CC7CC7C6)CC5)nc4c3F)c12] | 608.9 | 10.14 (s, 1H), 9.02 (s, 1H), 7.97 (dd, J = 9.2, 6.0 Hz, 1H), 7.46 (t, J = 9.2 Hz, 1H), 7.39 (d, J = 2.4 Hz, 1H), 7.17 (d, J = 2.4 Hz, 1H), 4.48 (d, J = 11.6 Hz, 1H), 4.33 − 4.14 (m, 3H), 3.92 (d, J = 1.2 Hz, 1H), 3.67 − 3.59 (m, 1H), 3.54 (d, J = 10.4 Hz, 3H), 2.96 (dd, J = 8.8, 4.8 Hz, 2H), 2.38 (q, J = 12.0 Hz, 2H), 2.23 (d, J = 8.8 Hz, 2H), 1.66 (s, 4H), 1.35 − 1.27 (m, 2H), 0.61 − 0.50 (m, 3H), 0.42 − 0.35 (m, 2H), 0.29 − 0.20 (m, 1H). |
| 14 | 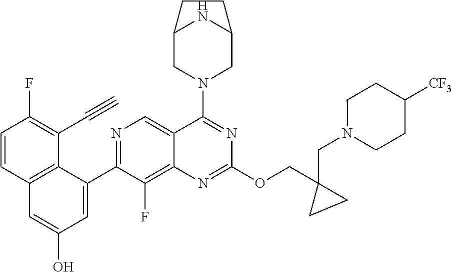 [SMILES: C#Cc1c(F)ccc2cc(O)cc(-c3ncc4c(N5CC6CCC(C5)N6)nc(OCC5(CN6CCC(C(F)(F)F)CC6)CC5)nc4c3F)c12] | 678.9 | 10.14 (s, 1H), 9.02 (s, 1H), 7.97 (dd, J = 9.2, 6.0 Hz, 1H), 7.46 (t, J = 9.2 Hz, 1H), 7.39 (d, J = 2.4 Hz, 1H), 7.16 (d, J = 2.4 Hz, 1H), 4.53 − 4.45 (m, 1H), 4.34 − 4.22 (m, 3H), 3.92 (d, J = 1.2 Hz, 1H), 3.66 − 3.60 (m, 1H), 3.59 − 3.51 (m, 3H), 3.03 (s, 2H), 2.39 − 2.26 (m, 2H), 2.26 − 2.14 (m, 1H), 1.94 − 1.82 (m, 2H), 1.78 − 1.63 (m, 6H), 1.43 − 1.31 (m, 2H), 0.65 (q, J = 2.8 Hz, 2H), 0.44 − 0.36 (m, 2H). |
| 15 | 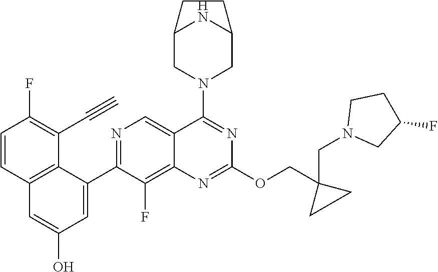 [SMILES: C#Cc1c(F)ccc2cc(O)cc(-c3ncc4c(N5CC6CCC(C5)N6)nc(OCC5(CN6CC[C@H](F)C6)CC5)nc4c3F)c12] | 615.0 | 10.17 (s, 1H), 9.03 (s, 1H), 7.97 (dd, J = 9.2, 6.0 Hz, 1H), 7.46 (t, J = 9.2 Hz, 1H), 7.39 (d, J = 2.4 Hz, 1H), 7.17 (d, J = 2.4 Hz, 1H), 5.24 (dd, J = 6.0 Hz, J = 6.0 Hz 1H), 4.49 (d, J = 12.0 Hz, 1H), 4.32 − 4.20 (m, 3H), 3.95 (d, J = 2.8 Hz, 1H), 3.66-3.60 (m, 1H), 3.57-3.51 (m, 3H), 2.84 − 2.76 (m, 2H), 2.67 − 2.59 (m, 2H), 2.46 − 2.30 (m, 3H), 2.10-2.01 (m, 1H), 1.90-1.75 (m, 1H), 1.67 (s, 4H), 0.63 (s, 2H), 0.44 (s, 2H). |
| 16 | 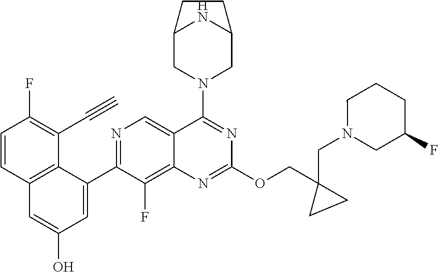 [SMILES: C#Cc1c(F)ccc2cc(O)cc(-c3ncc4c(N5CC6CCC(C5)N6)nc(OCC5(CN6CCC[C@@H](F)C6)CC5)nc4c3F)c12] | 629.0 | 10.16 (s, 1H), 9.03 (s, 1H), 7.98 (dd, J = 9.2, 6.0 Hz, 1H), 7.47 (t, J = 9.2 Hz, 1H), 7.39 (d, J = 2.4 Hz, 1H), 7.17 (d, J = 2.4 Hz, 1H), 4.65 − 4.49 (m, 2H), 4.33 − 4.17 (m, 3H), 3.94 (s, 1H), 3.65 (d, J = 12.0 Hz, 1H), 3.60 − 3.51 (m, 3H), 2.77 (s, 1H),2.56 − 2.50 (m, 1H), 2.35 (d, J = 6.0 Hz, 3H), 2.22 (d, J = 10.4 Hz, 1H), 1.80 (s, 1H), 1.67 (s, 5H), 1.53 − 1.37 (m, 2H), 1.23 (s, 1H), 0.65 (q, J = 4.0 Hz, 2H), 0.42 (s, 2H). |
| 17 | 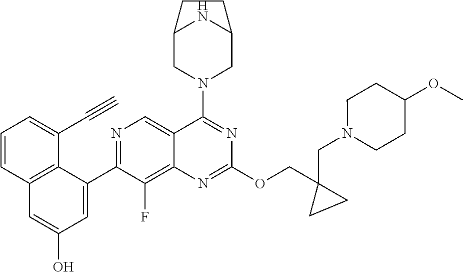 [SMILES: C#Cc1cccc2cc(O)cc(-c3ncc4c(N5CC6CCC(C5)N6)nc(OCC5(CN6CCC(OC)CC6)CC5)nc4c3F)c12] | 623.0 | 10.12 (s, 1H), 9.01 (s, 1H), 7.88 (dd, J = 8.0, 1.6 Hz, 1H), 7.49 − 7.38 (m, 2H), 7.33 (d, J = 2.4 Hz, 1H), 7.11 (d, J = 2.4 Hz, 1H), 4.52-4.46 (m, 1H), 4.33 − 4.19 (m, 3H), 3.64 (d, J = 12.0 Hz, 1H), 3.58 −3.51 (m, 4H), 3.19 (s, 3H), 3.12 (dt, J = 8.8, 4.8 Hz, 1H), 2.73 (s, 2H), 2.36 − 2.21 (m, 2H), 2.04 (t, J = 10.4 Hz, 2H), 1.81 − 1.75 (m, 2H), 1.70 − 1.62 (m, 4H), 1.43 − 1.27 (m, 2H), 0.63 (d, J = 5.2 Hz, 2H), 0.39 (t, J = 2.8 Hz, 2H). |
| 18 | 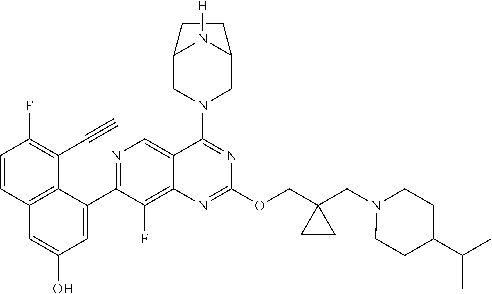 [SMILES: C#Cc1c(F)ccc2cc(O)cc(-c3ncc4c(N5CC6CCC(C5)N6)nc(OCC5(CN6CCC(C(C)C)CC6)CC5)nc4c3F)c12] | 653.1 | 10.15 (s, 1H), 9.02 (s, 1H), 7.97 (dd, J = 9.2, 6.0 Hz, 1H), 7.46 (t, J = 9.2 Hz, 1H), 7.39 (d, J = 2.4 Hz, 1H), 7.16 (d, J = 2.4 Hz, 1H), 4.51 −4.46 (m, 1H), 4.31 − 4.26 (m, 3H), 3.94 (s, 1H), 3.65 − 3.61 (m, 1H), 3.53 (d, J = 7.6 Hz, 3H), 2.97 (s, 2H), 2.33 − 2.27 (m, 2H), 2.25 − 2.21 (m, 2H), 1.75 (s, 4H), 1.66 (s, 2H), 1.57 − 1.51 (m, 1H), 1.34-1.24 (m, 2H), 1.09 − 0.93 (s, 1H), 0.80 (d, J = 6.8 Hz, 6H), 0.63 (s, 2H), 0.39 (s, 2H). |
| 19 | 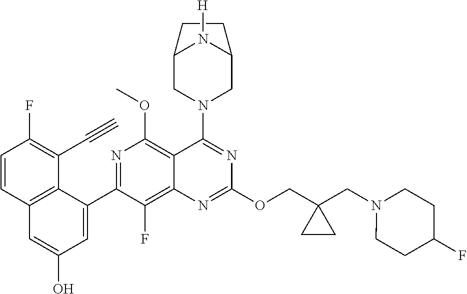 [SMILES: C#Cc1c(F)ccc2cc(O)cc(-c3nc(OC)c4c(N5CC6CCC(C5)N6)nc(OCC5(CN6CCC(F)CC6)CC5)nc4c3F)c12] | 659.0 | 10.15 (s, 1H), 7.97 (dd, J = 9.2, 6.0 Hz, 1H), 7.47 (t, J = 9.2 Hz, 1H), 7.38 (d, J = 2.4 Hz, 1H), 7.21 (d, J = 2.4 Hz, 1H), 4.88- 4.65 (m, 1H), 4.25 (s, 2H), 4.10 (s, 1H), 3.88 (d, J = 4.4 Hz, 5H), 3.64 − 3.43 (m, 2H), 3.40 − 3.35 (m, 1H), (s, 1H), 3.31 (s, 1H), 2.57 (s, 2H), 2.37 − 2.24 (m, 4H), 1.81 (t, J = 8.0 Hz, 2H), 1.64-55 (m, 6H), 0.63 (s, 2H), 0.44 − 0.37 (m, 2H). |
| 20 | 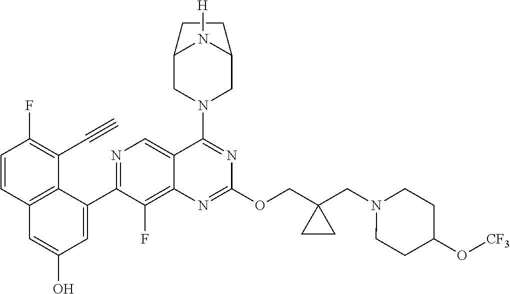 [SMILES: C#Cc1c(F)ccc2cc(O)cc(-c3ncc4c(N5CC6CCC(C5)N6)nc(OCC5(CN6CCC(OC(F)(F)F)CC6)CC5)nc4c3F)c12] | 694.9 | 9.04 (s, 1H), 8.21 (s, 1H), 7.98 (dd, J = 9.2, 6.0 Hz, 1H), 7.47 (t, J = 9.2 Hz, 1H), 7.39 (d, J = 2.4 Hz, 1H), 7.17 (d, J = 2.4 Hz, 1H), 4.53 −4.47 (m, 1H), 4.40 − 4.27 (s, 4H), 3.94 (d, J = 1.2 Hz, 1H), 3.66 − 3.50 (m, 4H), 2.71 (s, 2H), 2.37 − 2.21 (m, 4H), 1.90 − 1.85 (m, 2H), 1.77 − 1.57 (m, 6H), 0.64 (q, J = 3.7, 3.2 Hz, 2H), 0.45 − 0.38 (m, 2H). |
| 21 | 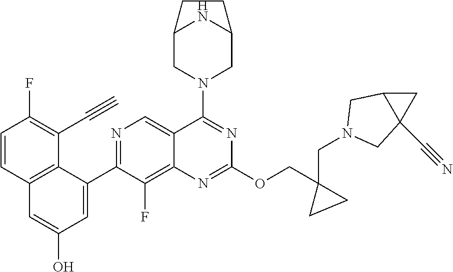 [SMILES: C#Cc1c(F)ccc2cc(O)cc(-c3ncc4c(N5CC6CCC(C5)N6)nc(OCC5(CN6CC7CC7(C#N)C6)CC5)nc4c3F)c12] | 633.9 | 10.14 (s, 1H), 9.02 (s, 1H), 7.97 (dd, J = 9.2, 5.9 Hz, 1H), 7.46 (t, J = 9.0 Hz, 1H), 7.39 (d, J = 2.6 Hz, 1H), 7.17 (d, J = 2.5 Hz, 1H), 4.47 (d, J = 11.8 Hz, 1H), 4.29 (d, J = 12.1 Hz, 1H), 4.21 (s, 2H), 3.91 (d, J = 1.0 Hz, 1H), 3.63 (d, J = 12.2 Hz, 1H), 3.55 (d, J = 9.6 Hz, 3H), 3.22 (d, J = 8.5 Hz, 1H), 2.99 (dd, J = 9.3, 2.3 Hz, 1H), 2.49 − 2.34 (m, 4H), 2.18 (ddd, J = 8.4, 5.0, 3.4 Hz, 1H), 1.66 (s, 4H), 1.31 (t, J = 4.8 Hz, 1H), 1.10 (dd, J = 8.3, 4.4 Hz, 1H), 0.59 (s, 2H), 0.42 (dd, J = 9.1, 6.3 Hz, 2H). |
| 22 | 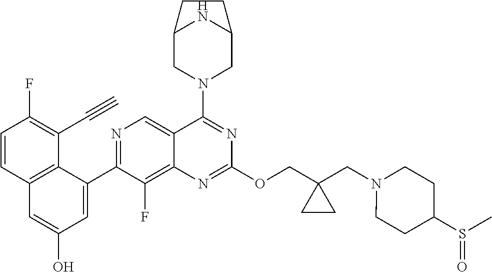 [SMILES: C#Cc1c(F)ccc2cc(O)cc(-c3ncc4c(N5CC6CCC(C5)N6)nc(OCC5(CN6CCC(S(C)=O)CC6)CC5)nc4c3F)c12] | 672.9 | 9.03 (s, 1H), 8.26 (s, 3H), 7.97 (dd, J = 9.2, 5.9 Hz, 1H), 7.46 (t, J = 9.0 Hz, 1H), 7.39 (d, J = 2.6 Hz, 1H), 7.18 (d, J = 2.6 Hz, 1H), 4.50 (d, J = 12.1 Hz, 1H), 4.33 − 4.21 (m, 3H), 3.94 (s, 1H), 3.66 (d, J = 12.1 Hz, 2H), 3.57 (d, J = 12.4 Hz, 2H), 3.55 (s, 1H), 3.04 (s, 2H), 2.46 (s, 3H), 2.45 (s, 2H), 1.87 (d, J = 12.5 Hz, 2H), 1.74 (d, J = 12.6 Hz, 1H), 1.69 (s, 5H), 1.58 − 1.41 (m, 2H), 0.65 (q, J = 3.0 Hz, 2H), 0.42 (s, 2H). |
| 23 | 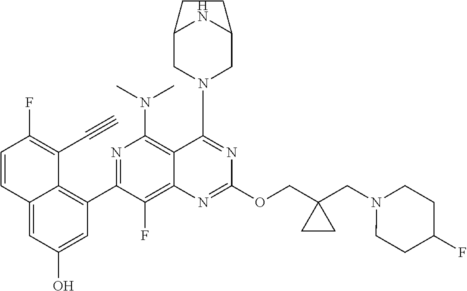 [SMILES: C#Cc1c(F)ccc2cc(O)cc(-c3nc(N(C)C)c4c(N5CC6CCC(C5)N6)nc(OCC5(CN6CCC(F)CC6)CC5)nc4c3F)c12] | 672.3 | 8.22 (s, 1H), 7.99-7.92 (m, 1H), 7.45 (t, J = 9.0 Hz, 1H), 7.35 (m, 1H), 7.31-7.27 (m, 1H), 5.04-4.91 (m, 1H), 4.73-4.54 (m, 1H), 4.33- 4.26 (m, 1H), 4.21-4.14 (m, 1H), 4.04-3.90 (m, 1H), 3.59-3.52 (m, 4H), 2.97-2.82 (m, 5H), 2.69 (m, 2H), 2.58 (m, 2H), 2.39-2.35 (m, 1H), 2.30-2.21 (m, 2H), 1.95-1.41 (m, 9H), 1.09-0.96 (m, 1H), 0.64 (m, 2H), 0.41 (s, 2H). |
| 24 | 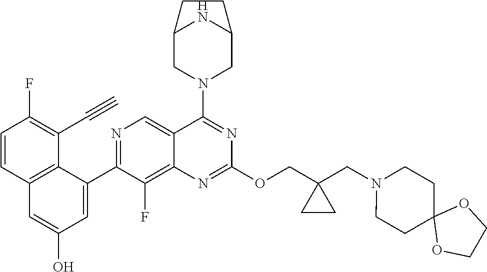 [SMILES: C#Cc1c(F)ccc2cc(O)cc(-c3ncc4c(N5CC6CCC(C5)N6)nc(OCC5(CN6CCC7(CC6)OCCO7)CC5)nc4c3F)c12] | 669.9 | 9.03 (s, 1H), 8.18 (s, 1H), 7.97 (dd, J = 9.2, 5.9 Hz, 1H), 7.46 (t, J = 9.0 Hz, 1H), 7.39 (d, J = 2.6 Hz, 1H), 7.18 (d, J = 2.6 Hz, 1H), 4.51 (d, J = 12.4 Hz, 1H), 4.38 (s, 5H), 3.92 (s, 1H), 3.83 (s, 4H), 3.70 − 3.61 (m, 4H), 3.58 (d, J = 12.5 Hz, 1H), 3.43 (t, J = 5.4 Hz, 1H), 2.36 (d, J = 12.5 Hz, 2H), 1.70 (s, 4H), 1.56 (t, J = 5.7 Hz, 4H), 0.64 (q, J = 3.5, 3.1 Hz, 2H), 0.41 (s, 2H). |
| 25 | 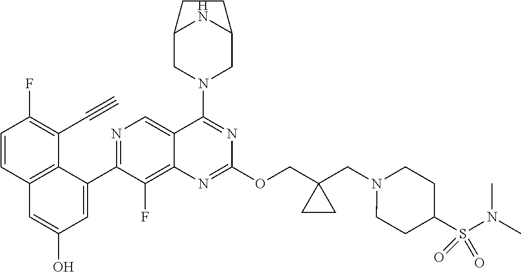 [SMILES: C#Cc1c(F)ccc2cc(O)cc(-c3ncc4c(N5CC6CCC(C5)N6)nc(OCC5(CN6CCC(S(=O)(=O)N(C)C)CC6)CC5)nc4c3F)c12] | 717.9 | 10.14 (s, 1H), 9.02 (s, 1H), 7.97 (dd, J = 9.2, 5.9 Hz, 1H), 7.46 (t, J = 9.0 Hz, 1H), 7.39 (d, J = 2.6 Hz, 1H), 7.18 (d, J = 2.6 Hz, 1H), 4.52 − 4.43 (m, 1H), 4.38 (t, J = 5.6 Hz, 1H), 4.33 − 4.21 (m, 1H), 4.15 (m, 1H), 3.93 (d, J = 1.1 Hz, 1H), 3.64 (d, J = 12.2 Hz, 1H), 3.55 (d, J = 11.8 Hz, 3H), 3.04 (d, J = 10.4 Hz, 1H), 2.82 (d, J = 2.6 Hz, 2H), 2.79 (s, 6H), 2.35 (d, J = 12.7 Hz, 1H), 2.27 (d, J = 12.9 Hz, 1H), 2.23(m,1H)1.95 − 1.81 (m, 4H), 1.67 (s, 4H), 1.57 (q, J = 12.8, 12.3 Hz, 2H), 0.64 (s, 2H), 0.43 − 0.38 (m, 2H). |
| 26 | 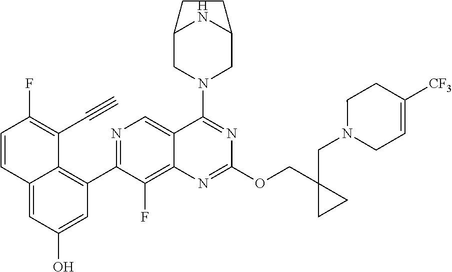 [SMILES: C#Cc1c(F)ccc2cc(O)cc(-c3ncc4c(N5CC6CCC(C5)N6)nc(OCC5(CN6CC=C(C(F)(F)F)CC6)CC5)nc4c3F)c12] | 676.9 | 10.16 (s, 1H), 9.02 (s, 1H), 7.97 (dd, J = 9.2, 5.9 Hz, 1H), 7.46 (t, J = 9.0 Hz, 1H), 7.38 (d, J = 2.6 Hz, 1H), 7.17 (d, J = 2.6 Hz, 1H), 6.42 − 6.39 (m, 1H), 4.50 − 4.45 (m, 1H), 4.30 − 4.25 (m, 3H), 3.92 (s, 1H), 3.65 − 3.60 (m, 1H), 3.53 (d, J = 7.9 Hz, 3H), 3.09 (p, J = 3.1 Hz, 2H), 2.65 (q, J = 6.7, 5.8 Hz, 2H), 2.47 − 2.36 (m, 2H), 2.18 (s, 2H), 1.65 (s, 4H), 0.74 − 0.55 (m, 2H), 0.53 − 0.33 (m, 2H). |
| 27 | 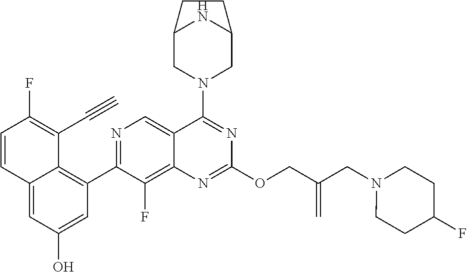 [SMILES: C#Cc1c(F)ccc2cc(O)cc(-c3ncc4c(N5CC6CCC(C5)N6)nc(OCC(=C)CN5CCC(F)CC5)nc4c3F)c12] | 614.7 | 10.42 − 10.02 (m, 1H), 9.07 (s, 1H), 8.05 − 7.90 (m, 1H), 7.47 (t, J = 9.0 Hz, 1H), 7.40 (s, 1H), 7.19 (s, 1H), 5.21 (s, 1H), 5.14 (s, 1H), 4.93 (s, 2H), 4.72 (d, J = 7.4 Hz, 1H), 4.64 − 4.51 (m, 1H), 4.45 − 4.35 (m, 1H), 3.91 (d, J = 6.2 Hz, 2H), 3.83 − 3.76 (m, 2H), 3.65 − 3.57 (m, 2H), 3.02 (s, 2H), 2.48 (s, 1H), 2.28 (t, J = 9.3 Hz, 2H), 1.92 − 1.74 (m, 6H), 1.74 − 1.59 (m, 2H). |
| 28 | 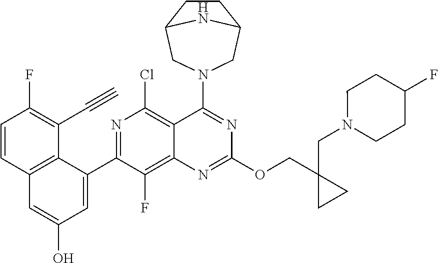 [SMILES: C#Cc1c(F)ccc2cc(O)cc(-c3nc(Cl)c4c(N5CC6CCC(C5)N6)nc(OCC5(CN6CCC(F)CC6)CC5)nc4c3F)c12] | 663.6 | 8.24 (s, 1H), 7.99 (dd, J = 5.8, 9.2 Hz, 1H), 7.48 (t, J = 8.8 Hz, 1H), 7.41 (d, J = 2.4 Hz, 1H), 7.25 (s, 1H), 4.72-4.55 (m, 1H), 4.31 (s, 2H), 3.83 (s, 8H), 2.54 (d, J = 4.0 Hz, 2H), 2.37-2.23 (m, 4H), 1.91- 1.70 (m, 3H), 1.62 (s, 4H), 0.64 (s, 2H), 0.41 (s, 1H). |
| 29 | 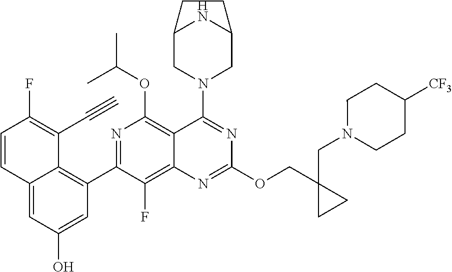 [SMILES: C#Cc1c(F)ccc2cc(O)cc(-c3nc(OC(C)C)c4c(N5CC6CCC(C5)N6)nc(OCC5(CN6CCC(C(F)(F)F)CC6)CC5)nc4c3F)c12] | 737.5 | 8.23 (s, 1H), 8.00-7.93 (m, 1H), 7.46 (t, J = 9.0 Hz, 1H), 7.38 (d, J = 2.4 Hz, 1H), 7.19 (d, J = 2.4 Hz, 1H), 5.36-5.24 (m, 1H), 4.27 (s, 2H), 3.92-3.83 (m, 2H), 3.64- 3.54 (m, 5H), 3.06-2.97 (m, 3H), 2.38-2.25 (m, 3H), 1.93-1.82 (m, 2H), 1.74-1.69 (m, 2H), 1.62 (s, 3H), 1.44-1.35 (m, 2H), 1.31 (t, J = 6.0 Hz, 6H), 0.64 (s, 2H), 0.41 (s, 2H). |
| 30 | 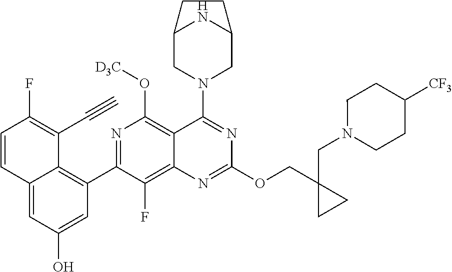 [SMILES: [2H]C([2H])([2H])Oc1nc(-c2cc(O)cc3ccc(F)c(C#C)c23)c(F)c2nc(OCC3(CN4CCC(C(F)(F)F)CC4)CC3)nc(N3CC4CCC(C3)N4)c12] | 712.7 | 7.96-7.90 (m, 1H), 7.47-7.40 (m, 1H), 7.35-7.30 (m, 1H), 7.18 (s, 1H), 4.26 (s, 2H), 4.14-4.06 (m, 1H), 3.89-3.83 (m, 1H), 3.48-3.44 (m, 2H), 3.19-3.15 (m, 2H), 3.06- 3.01 (m, 2H), 2.31-2.26 (m, 2H), 1.91-1.84 (m, 2H), 1.74 (d, J = 10.6 Hz, 2H), 1.61-1.55 (m, 4H), 1.43-1.36 (m, 2H), 1.35-1.28 (m, 2H), 0.64 (s, 2H), 0.41 (s, 2H). |
| 31 | 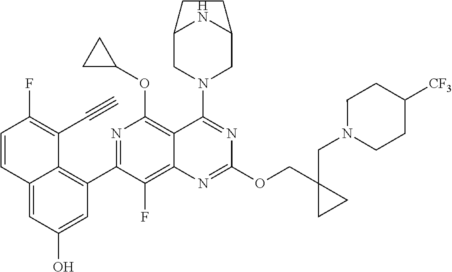 [SMILES: C#Cc1c(F)ccc2cc(O)cc(-c3nc(OC4CC4)c4c(N5CC6CCC(C5)N6)nc(OCC5(CN6CCC(C(F)(F)F)CC6)CC5)nc4c3F)c12] | 735.4 | 8.23 (s, 1H), 7.96 (dd, J = 5.8, 9.2 Hz, 1H), 7.46 (t, J = 9.0 Hz, 1H), 7.38 (d, J = 2.4 Hz, 1H), 7.21 (d, J = 2.4 Hz, 1H), 4.28-4.23 (m, 3H), 4.08-3.93 (m, 2H), 3.88 (s, 1H), 3.52 (d, J = 9.0 Hz, 4H), 3.05-2.97 (m, 3H), 2.30 ( d, J = 9.6 Hz, 2H), 2.22-2.16 (m, 1H), 1.90-1.83 (m, 2H), 1.72 (d, J = 11.8 Hz, 2H), 1.59 (s, 4H), 1.40- 1.32 (m, 2H), 0.74-0.66 (m, 4H), 0.64-0.61 (m, 2H), 0.40 (s, 2H). |
| 32 | 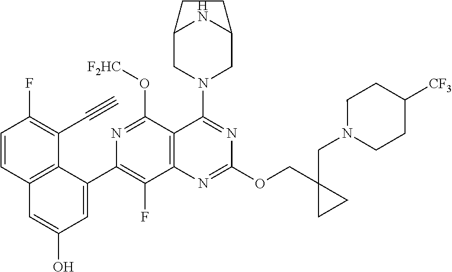 [SMILES: C#Cc1c(F)ccc2cc(O)cc(-c3nc(OC(F)F)c4c(N5CC6CCC(C5)N6)nc(OCC5(CN6CCC(C(F)(F)F)CC6)CC5)nc4c3F)c12] | 745.4 | 8.22 (s, 1H), 7.98 (dd, J = 6.0, 9.0 Hz, 1H), 7.88-7.44 (m, 2H), 7.40 (d, J = 2.4 Hz, 1H), 7.23 (d, J = 2.4 Hz, 1H), 4.35-4.25 (m, 2H), 4.24-4.00 (m, 2H), 3.96 (s, 1H), 3.54 (d, J = 9.4 Hz, 3H), 3.46 (d, J = 13.6 Hz, 3H), 3.01 (d, J = 10.4 Hz, 2H), 2.36-2.26 (m, 2H), 2.24-2.15 (m, 1H), 1.93-1.82 (m, 2H), 1.73 (d, J = 11.4 Hz, 2H), 1.62 (s, 2H), 1.42-1.30 (m, 2H), 0.64 (s, 2H), 0.41 (s, 2H). |
| 33 | 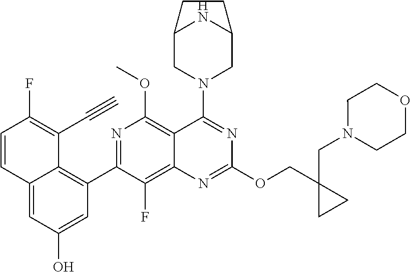 [SMILES: C#Cc1c(F)ccc2cc(O)cc(-c3nc(OC)c4c(N5CC6CCC(C5)N6)nc(OCC5(CN6CCOCC6)CC5)nc4c3F)c12] | 642.9 | 10.04 (s, 1H), 8.14 (td, J = 7.9, 7.3, 1.4 Hz, 1H), 7.76 − 7.66 (m, 2H), 7.63 − 7.52 (m, 1H),, 4.58 − 4.49 (m, 2H), 4.34 − 4.28 (m, 1H), 3.75 − 3.61 (m, 10H), 3.54 − 3.45 (m, 2H), 2.87 − 2.34 (m, 4H), 2.70 − 2.59 (m, 2H), 1.81 (s, 4H), 1.66 (s, 1H), 0.62 (q, J = 3.4 Hz, 1H), 0.44 (t, J = 3.0 Hz, 2H). |
| 34 | 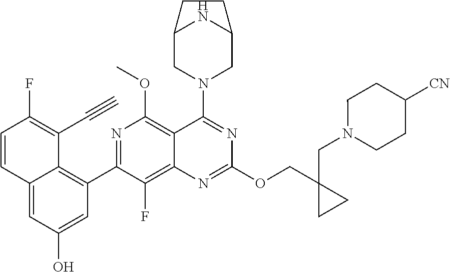 [SMILES: C#Cc1c(F)ccc2cc(O)cc(-c3nc(OC)c4c(N5CC6CCC(C5)N6)nc(OCC5(CN6CCC(C#N)CC6)CC5)nc4c3F)c12] | 655.9 | 10.04 (s, 1H), 8.14 (td, J = 7.9, 7.3, 1.4 Hz, 1H), 7.76 − 7.66 (m, 2H), 7.63 − 7.52 (m, 1H),, 4.58 − 4.49 (m, 2H), 4.34 − 4.28 (m, 5H), 3.75 − 3.61 (m, 2H), 3.54 − 3.45 (m, 2H), 2.97 − 2.84 (m, 1H), 2.70 − 2.59 (m, 2H), 2.45 − 2.37 (m, 3H), 1.81 (s, 2H), 1.66 (s, 6H), 0.62 (q, J = 3.4 Hz, 1H), 0.44 (t, J = 3.0 Hz, 1H). |
| 35 | 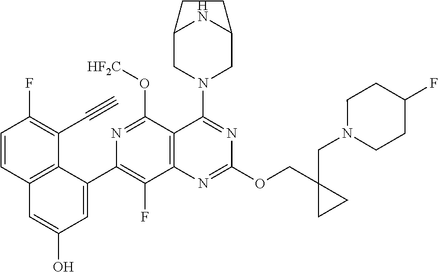 | 695.5 | 8.22-8.17 (m, 1H), 8.02-7.94 (m, 1H), 7.45 (s, 2H), 7.42-7.38 (m, 1H), 7.27-7.20 (m, 1H), 4.73-4.54 (m, 1H), 4.31-4.26 (m, 2H), 3.98- 3.96 (m, 1H), 3.55-3.48 (m, 7H), 2.59-2.53 (m, 2H), 2.33-2.27 (m, 4H), 1.86-1.75 (m, 2H), 1.69-1.57 (m, 6H), 0.66-0.57 (m, 2H), 0.44- 0.37 (m, 2H). |
| 36 | 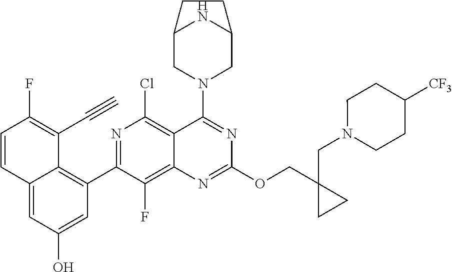 [SMILES: C#Cc1c(F)ccc2cc(O)cc(-c3nc(Cl)c4c(N5CC6CCC(C5)N6)nc(OCC5(CN6CCC(C(F)(F)F)CC6)CC5)nc4c3F)c12] | 713.6 | 8.21 (s, 1H), 7.99 (dd, J = 6.2, 9.0 Hz, 1H), 7.48 (t, J = 9.0 Hz, 1H), 7.41 (d, J = 2.4 Hz, 1H), 7.24 (s, 1H), 4.31 (s, 2H), 3.92-3.76 (m, 2H), 3.58-3.45 (m, 6H), 3.05-2.98 (m, 2H), 2.54 (s, 1H), 2.38-2.33 (m, 1H), 2.28-2.16 (m, 2H), 1.91- 1.83 (m, 2H), 1.73 (d, J = 11.2 Hz, 2H), 1.57 (d, J = 2.8 Hz, 2H), 1.42-1.31 (m, 2H), 0.64 (s, 2H), 0.41 (s, 2H). |
| 37 | 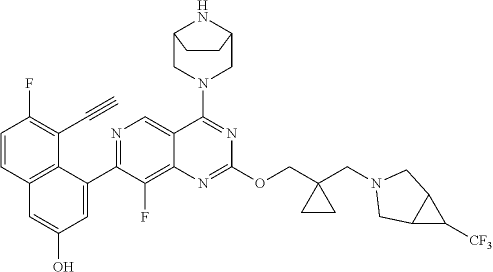 [SMILES: C#Cc1c(F)ccc2cc(O)cc(-c3ncc4c(N5CC6CCC(C5)N6)nc(OCC5(CN6CC7C(C6)C7C(F)(F)F)CC5)nc4c3F)c12] | 676.7 | 10.16 (s, 1H), 9.02 (s, 1H), 7.97 (dd, J = 9.2, 5.9 Hz, 1H), 7.46 (t, J = 9.0 Hz, 1H), 7.39 (d, J = 2.5 Hz, 1H), 7.16 (d, J = 2.5 Hz, 1H), 4.53 − 4.41 (m, 1H), 4.34 − 4.15 (m, 3H), 3.91 (d, J = 1.0 Hz, 1H), 3.65 − 3.60 (m, 1H), 3.58 − 3.52 (m, 3H), 3.10 − 3.00 (m, 2H), 2.47 (s, 1H), 2.37 − 2.24 (m, 3H), 1.81 (s, 3H), 1.65 (s, 4H), 0.59 (d, J = 4.8 Hz, 2H), 0.40 (s, 2H). |
| 38 | 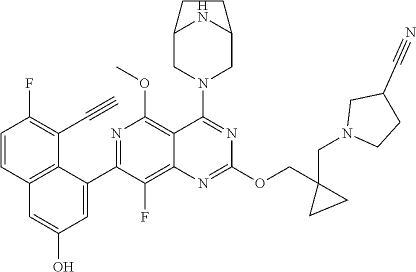 [SMILES: C#Cc1c(F)ccc2cc(O)cc(-c3nc(OC)c4c(N5CC6CCC(C5)N6)nc(OCC5(CN6CCC(C#N)C6)CC5)nc4c3F)c12] | 651.8 | 10.14 (s, 1H), 7.97 (dd, J = 9.2, 5.9 Hz, 1H), 7.46 (t, J = 9.0 Hz, 1H), 7.38 (d, J = 2.6 Hz, 1H), 7.22 (d, J = 2.5 Hz, 1H), 4.34 − 4.25 (m, 1H), 4.23 − 4.17 (m, 1H), 4.11 (s, 1H), 3.88 (d, J = 3.8 Hz, 5H), 3.47 (s, 2H), 3.40 (s, 1H), 3.36 (s, 1H), 3.25-3.18 (m, 1H), 2.81 − 2.73 (m, 1H), 2.72 − 2.63 (m, 2H), 2.54 (s, 1H), 2.46 (s, 1H), 2.45 − 2.33 (m, 1H), 2.21 − 2.08 (m, 1H), 1.95 − 1.84 (m, 1H), 1.70 − 1.49 (m, 4H), 0.63 (d, J = 4.5 Hz, 2H), 0.44 (q, J = 3.9 Hz, 2H). |
| 39 | 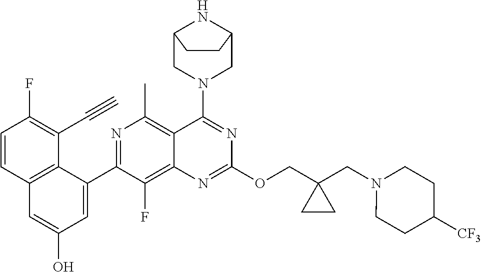 [SMILES: C#Cc1c(F)ccc2cc(O)cc(-c3nc(C)c4c(N5CC6CCC(C5)N6)nc(OCC5(CN6CCC(C(F)(F)F)CC6)CC5)nc4c3F)c12] | 693.3 | 10.11 (s, 1H), 7.97 (dd, J = 9.2, 5.9 Hz, 1H), 7.46 (t, J = 9.0 Hz, 1H), 7.38 (d, J = 2.6 Hz, 1H), 7.20 (d, J = 2.5 Hz, 1H), 4.32 − 4.25 (m, 2H), 3.61 (s, 2H), 3.44 (s, 3H), 3.32 (s, 4H), 3.07 − 2.95 (m, 2H), 2.40 − 2.31 (m, 2H), 2.29 − 2.24 (m, 2H), 2.23 − 2.10 (m, 1H), 1.91 − 1.81 (m, 2H), 1.78 − 1.72 (m, 2H), 1.53 (s, 3H), 1.39 − 1.31 (m, 2H), 0.63 (t, J = 2.9 Hz, 2H), 0.44 − 0.37 (m, 2H). |
| 40 | 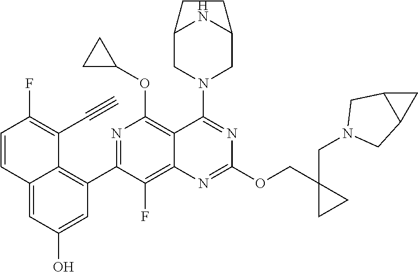 [SMILES: C#Cc1c(F)ccc2cc(O)cc(-c3nc(OC4CC4)c4c(N5CC6CCC(C5)N6)nc(OCC5(CN6CC7CC7C6)CC5)nc4c3F)c12] | 665.5 | 8.23 (s, 1H), 7.97 (dd, J = 6.0, 9.0 Hz, 1H), 7.46 (t, J = 9.0 Hz, 1H), 7.38 (d, J = 2.4 Hz, 1H), 7.22 (d, J = 2.4 Hz, 1H), 4.20 (d, J = 2.6 Hz, 2H), 3.91 (s, 1H), 3.63 (d, J = 9.8 Hz, 4H), 3.42 (td, J = 6.6, 8.8 Hz, 4H), 2.95 (dd, J = 4.6, 8.6 Hz, 2H), 2.43-2.30 (m, 3H), 2.23 (d, J = 8.0 Hz, 2H), 1.70-1.57 (m, 4H), 1.34-1.29 (m, 2H), 0.77-0.67 (m, 4H), 0.58-0.53 (m, 3H), 0.40- 0.35 (m, 2H), 0.25 (dt, J = 3.6, 7.6 Hz, 1H). |
| 41 | 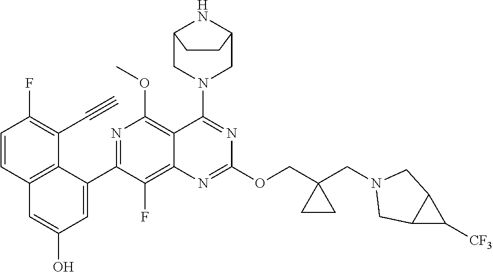 [SMILES: C#Cc1c(F)ccc2cc(O)cc(-c3nc(OC)c4c(N5CC6CCC(C5)N6)nc(OCC5(CN6CC7C(C6)C7C(F)(F)F)CC5)nc4c3F)c12] | 706.7 | 10.15 (s, 1H), 7.97 (dd, J = 9.2, 5.9 Hz, 1H), 7.46 (t, J = 9.0 Hz, 1H), 7.38 (d, J = 2.6 Hz, 1H), 7.20 (d, J = 2.6 Hz, 1H), 4.30 − 4.18 (m, 2H), 4.16 − 4.01 (m, 1H), 3.86 (d, J = 7.5 Hz, 5H), 3.48 (d, J = 6.5 Hz, 2H), 3.41 (s, 1H), 3.10 − 3.00 (m, 2H), 2.46 (s, 2H), 2.37 − 2.24 (m, 3H), 1.82 (s, 3H), 1.64 − 1.51 (m, 4H), 0.59 (d, J = 5.2 Hz, 2H), 0.40 (s, 2H). |
| 42 | 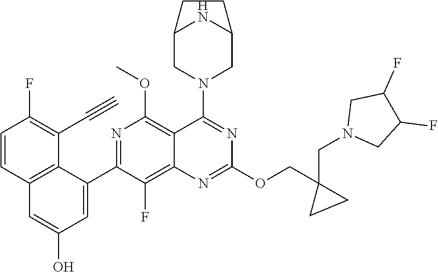 [SMILES: C#Cc1c(F)ccc2cc(O)cc(-c3nc(OC)c4c(N5CC6CCC(C5)N6)nc(OCC5(CN6CC(F)C(F)C6)CC5)nc4c3F)c12] | 662.9 | 10.13 (s, 1H), 7.97 (dd, J = 9.2, 5.9 Hz, 1H), 7.46 (t, J = 9.0 Hz, 1H), 7.38 (d, J = 2.6 Hz, 1H), 7.21 (d, J = 2.6 Hz, 1H), 5.28 − 5.17 (m, 1H), 5.17 − 5.08 (m, 1H), 4.31 − 4.25 (m, 1H), 4.19- 4.09 (m, 2H), 3.88 (d, J = 2.8 Hz, 5H), 3.46 (s, 2H), 3.12 − 3.05 (m, 2H), 3.05-2.99 (m, 2H), 2.75 − 2.69 (m, 1H), 2.67 (dd, J = 3.9, 2.0 Hz, 2H), 2.42 − 2.30 (m, 2H), 1.69 − 1.45 (m, 4H), 0.63 (s, 2H), 0.45 (s, 2H). |
| 43 | 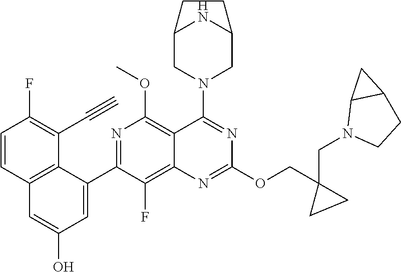 [SMILES: C#Cc1c(F)ccc2cc(O)cc(-c3nc(OC)c4c(N5CC6CCC(C5)N6)nc(OCC5(CN6CCC7CC76)CC5)nc4c3F)c12] | 638.9 | 10.13 (s, 1H), 7.97 (dd, J = 9.2, 5.9 Hz, 1H), 7.46 (t, J = 9.0 Hz, 1H), 7.38 (d, J = 2.6 Hz, 1H), 7.22 (d, J = 2.6 Hz, 1H), 4.37 − 4.27 (m, 1H), 4.26 − 4.17 (m, 1H), 4.16 − 4.03 (m, 1H), 3.88 (d, J = 3.1 Hz, 5H), 3.47 (s, 2H), 3.39 (s, 1H), 3.31 (s, 2H), 2.85 (s, 1H), 2.72 − 2.63 (m, 1H), 2.46 − 2.29 (m, 2H), 1.86 − 1.70 (m, 3H), 1.68 − 1.50 (m, 4H), 1.37 − 1.27 (m, 1H), 0.68 − 0.53 (m, 3H), 0.53 − 0.47 (m, 1H), 0.46 0.37 (m, 1H). |
| 44 | 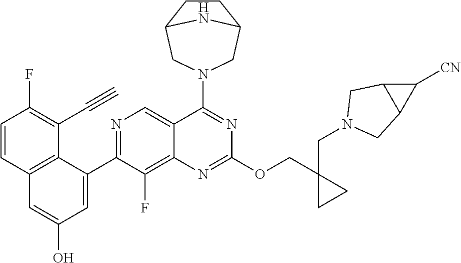 [SMILES: C#Cc1c(F)ccc2cc(O)cc(-c3ncc4c(N5CC6CCC(C5)N6)nc(OCC5(CN6CC7C(C#N)C7C6)CC5)nc4c3F)c12] | 633.9 | 10.13 (s, 1H), 9.02 (s, 1H), 7.97 (dd, J = 9.2, 5.9 Hz, 1H), 7.46 (t, J = 9.0 Hz, 1H), 7.38 (d, J = 2.6 Hz, 1H), 7.17 (d, J = 2.6 Hz, 1H), 4.47 (d, J = 12.1 Hz, 1H), 4.30 (d, J = 12.1 Hz, 1H), 4.22 (d, J = 2.5 Hz, 2H), 3.90 (s, 1H), 3.63 (d, J = 12.1 Hz, 1H), 3.55 (d, J = 4.3 Hz, 3H), 3.06 (dd, J = 9.5, 5.0 Hz, 2H), 2.43 − 2.33 (m, 2H), 2.28 (d, J = 9.4 Hz, 2H), 2.10 (d, J = 2.6 Hz, 2H), 1.75 (t, J = 3.3 Hz, 1H), 1.66 (s, 4H), 0.58 (s, 2H), 0.39 (s, 2H). |
| 45 | 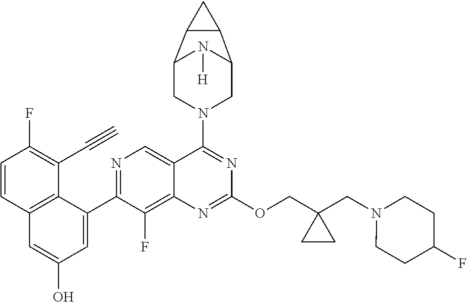 [SMILES: C#Cc1c(F)ccc2cc(O)cc(-c3ncc4c(N5CC6NC(C5)C5(C)CC65)nc(OCC5(CN6CCC(F)CC6)CC5)nc4c3F)c12] | 641.2 | 10.14 (s, 1H), 9.08 (s, 1H), 7.97 (dd, J = 9.2, 5.9 Hz, 1H), 7.46 (t, J = 9.0 Hz, 1H), 7.39 (d, J = 2.6 Hz, 1H), 7.16 (d, J = 2.6 Hz, 1H), 4.60 (d, J = 13.1 Hz, 2H), 4.34 (d, J = 12.1 Hz, 1H), 4.27 (d, J = 2.1 Hz, 2H), 3.96 (s, 1H), 3.71 (d, J = 12.0 Hz, 1H), 3.57 (s, 1H), 3.23 (d, J = 2.2 Hz, 2H), 2.67 (p, J = 2.0 Hz, 2H), 2.37 − 2.23 (m, 4H), 1.86 − 1.74 (m, 2H), 1.65 (s, 2H), 1.22 (dt, J = 7.5, 3.6 Hz, 2H), 0.64 (s, 2H), 0.40 (d, J = 4.9 Hz, 3H), 0.21 (td, J = 7.3, 5.1 Hz, 1H). |
| 46 | 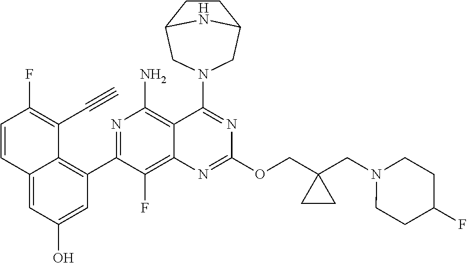 [SMILES: C#Cc1c(F)ccc2cc(O)cc(-c3nc(N)c4c(N5CC6CCC(C5)N6)nc(OCC5(CN6CCC(F)CC6)CC5)nc4c3F)c12] | 644.4 | 8.20 (s, 1H), 7.92 (dd, J = 6.0, 9.2 Hz, 1H), 7.42 (t, J = 9.2 Hz, 1H), 7.31 (d, J = 2.4 Hz, 1H), 7.16 (d, J = 2.0 Hz, 1H), 6.77-6.35 (m, 2H), 4.72-4.53 (m, 1H), 4.42-4.10 (m, 4H), 2.61-2.53 (m, 6H), 2.36- 2.23 (m, 5H), 1.87-1.72 (m, 3H), 1.71-1.57 (m, 4H), 1.35-1.20 (m, 1H), 0.62 (s, 1H), 0.40 (s, 1H). |
| 47 | 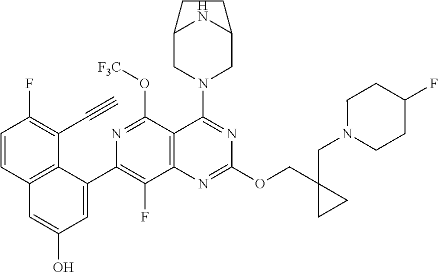 [SMILES: C#Cc1c(F)ccc2cc(O)cc(-c3nc(OC(F)(F)F)c4c(N5CC6CCC(C5)N6)nc(OCC5(CN6CCC(F)CC6)CC5)nc4c3F)c12] | 713.5 | 8.23 (s, 1H), 7.99 (dd, J = 6.0, 9.2 Hz, 1H), 7.48 (t, J = 9.0 Hz, 1H), 7.42 (d, J = 2.4 Hz, 1H), 7.22 (d, J = 2.4 Hz, 1H), 4.72-4.55 (m, 1H), 4.31 (s, 2H), 3.97 (s, 1H), 3.59-3.47 (m, 8H), 2.56 (br d, J = 4.6 Hz, 2H), 2.36-2.26 (m, 4H), 1.86-1.74 (m, 2H), 1.70-1.47 (m, 6H), 0.68-0.59 (m, 2H), 0.45-0.37 (m, 2H). |
| 48 | 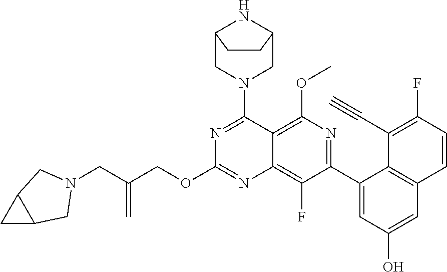 [SMILES: C#Cc1c(F)ccc2cc(O)cc(-c3nc(OC)c4c(N5CC6CCC(C5)N6)nc(OCC(=C)CN5CC6CC6C5)nc4c3F)c12] | 624.7 | 10.14 (s, 1H), 7.97 (dd, J = 9.2, 5.9 Hz, 1H), 7.46 (t, J = 9.0 Hz, 1H), 7.38 (d, J = 2.5 Hz, 1H), 7.22 (d, J = 2.5 Hz, 1H), 5.12 − 5.06 (m, 2H), 4.85 (s, 2H), 4.13 − 4.05 (m, 1H), 3.87 (d, J = 5.8 Hz, 5H), 3.47 (s, 2H), 3.44 − 3.36 (m, 2H), 3.10 (s, 2H), 2.89 (d, J = 8.6 Hz, 2H), 2.26 − 2.18 (m, 2H), 2.07 (s, 1H), 1.66 − 1.48 (m, 4H), 1.39 − 1.29 (m, 2H), 0.58 (q, J = 3.7 Hz, 1H), 0.28 (td, J = 7.7, 3.8 Hz, 1H). |
| 49 | 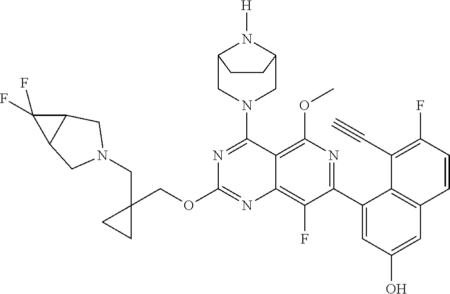 [SMILES: C#Cc1c(F)ccc2cc(O)cc(-c3nc(OC)c4c(N5CC6CCC(C5)N6)nc(OCC5(CN6CC7C(C6)C7(F)F)CC5)nc4c3F)c12] | 674.7 | 10.13 (s, 1H), 7.97 (dd, J = 9.2, 5.9 Hz, 1H), 7.46 (t, J = 9.0 Hz, 1H), 7.38 (d, J = 2.6 Hz, 1H), 7.21 (d, J = 2.6 Hz, 1H) , 4.20 (d, J = 2.0 Hz, 2H), 4.10 (s, 1H), 3.88 (s, 5H), 3.50 (s, 3H), 3.07 (m, 2H), 2.76 (d, J = 10.0 Hz, 2H), 2.42 (p, J = 1.9 Hz, 2H), 2.27 (m, 2H), 1.57 (d, J = 29.0 Hz, 5H), 0.59 (d, J = 4.9 Hz, 2H), 0.41 (q, J = 4.2 Hz, 2H). |
| 50 | 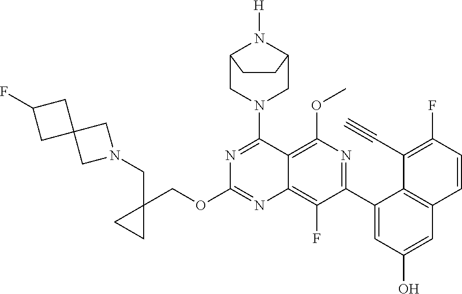 [SMILES: C#Cc1c(F)ccc2cc(O)cc(-c3nc(OC)c4c(N5CC6CCC(C5)N6)nc(OCC5(CN6CC7(CC(F)C7)C6)CC5)nc4c3F)c12] | 670.9 | 10.14 (s, 1H), 7.97 (dd, J = 9.2, 5.9 Hz, 1H), 7.46 (t, J = 9.0 Hz, 1H), 7.38 (d, J = 2.6 Hz, 1H), 7.22 (d, J = 2.6 Hz, 1H), 4.98- 4.83 (m, 1H), 4.16 (s, 2H), 4.11 (s, 1H), 3.88 (d, J = 4.6 Hz, 5H), 3.47 (s, 2H), 3.42-3.36 (m, 1H), 3.29 (s, 1H), 3.11 (d, J = 8.0 Hz, 4H), 2.48 − 2.41 (m, 2H), 2.39 − 2.28 (m, 2H), 2.24 − 2.12 (m, 2H), 1.70-1.50 (m, 4H), 0.49 (d, J = 4.4 Hz, 2H), 0.40 (t, J = 2.8 Hz, 2H). |
| 51 | 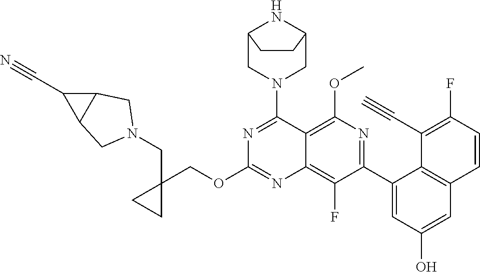 [SMILES: C#Cc1c(F)ccc2cc(O)cc(-c3nc(OC)c4c(N5CC6CCC(C5)N6)nc(OCC5(CN6CC7C(C#N)C7C6)CC5)nc4c3F)c12] | 664.6 | 10.13 (s, 1H), 7.97 (dd, J = 9.2, 5.9 Hz, 1H), 7.46 (t, J = 9.0 Hz, 1H), 7.38 (d, J = 2.6 Hz, 1H), 7.22 (d, J = 2.6 Hz, 1H), 4.26 − 4.15 (m, 2H), 4.10 (s, 1H), 3.87 (d, J = 6.7 Hz,5H), 3.51 (s, 3H), 3.40 (d, J = 9.5 Hz, 2H) 3.05 (dd, J = 9.4, 4.0 Hz, 2H), 2.39 (d, J = 10.7 Hz, 2H), 2.28 (d, J = 9.5 Hz, 2H), 2.10 (d, J = 2.7 Hz, 2H), 1.63 (s, 4H), 1.56 (s, 4H), 0.57 (d, J = 4.6 Hz, 2H), 0.41 − 0.36 (m, 2H). |
| 52 | 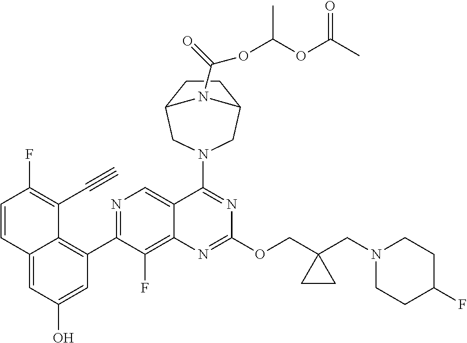 [SMILES: C#Cc1c(F)ccc2cc(O)cc(-c3ncc4c(N5CC6CCC(C5)N6C(=O)OC(C)OC(C)=O)nc(OCC5(CN6CCC(F)CC6)CC5)nc4c3F)c12] | 758.9 | 10.15 (s, 1H), 9.05 (s, 1H), 7.98 (dd, J = 9.2, 5.9 Hz, 1H), 7.46 (t, J = 9.0 Hz, 1H), 7.39 (d, J = 2.6 Hz, 1H), 7.17 (d, J = 2.6 Hz, 1H), 6.80 − 6.69 (m, 1H), 4.77 − 4.53 (m, 2H), 4.43 − 4.38 (m, 2H), 4.34 − 4.22 (m, 2H), 3.93 (d, J = 1.1 Hz, 1H), 3.72 − 3.55 (m, 2H), 2.56 (s, 2H), 2.37 − 2.22 (m, 4H), 2.06 (s, 3H), 1.95 − 1.68 (m, 6H), 1.65 (s, 2H), 1.48 (s, 3H), 1.24 (s, 4H), 0.89 − 0.81 (m, 1H), 0.65 (q, J = 3.4, 3.0 Hz, 2H), 0.49 − 0.34 (m, 2H). |

A compound described herein may also selected from any one of the following

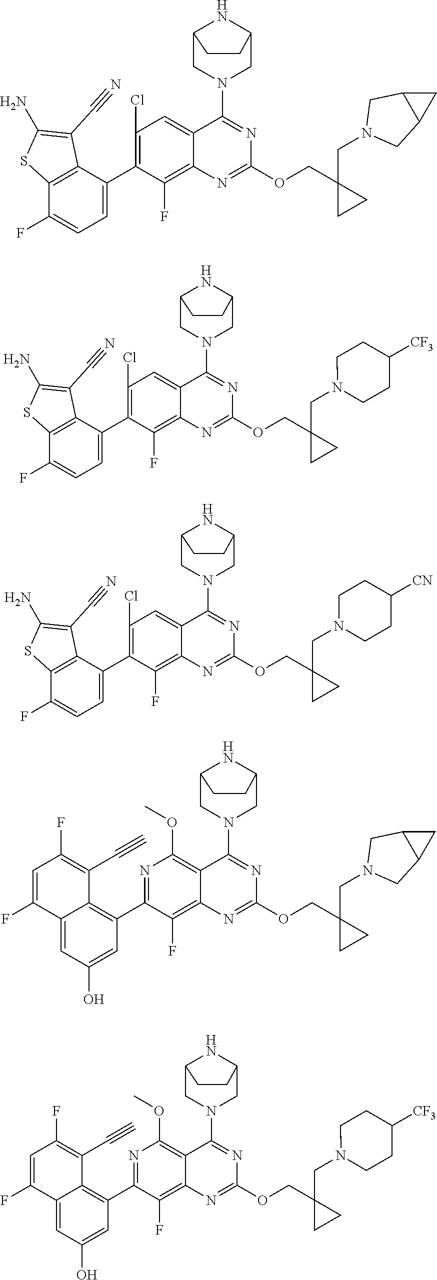 [SMILES: N#Cc1c(N)sc2c(F)ccc(-c3c(Cl)cc4c(N5CC6CCC(C5)N6)nc(OCC5(CN6CC7CC7C6)CC5)nc4c3F)c12 ; N#Cc1c(N)sc2c(F)ccc(-c3c(Cl)cc4c(N5CC6CCC(C5)N6)nc(OCC5(CN6CCC(C(F)(F)F)CC6)CC5)nc4c3F)c12 ; N#Cc1c(N)sc2c(F)ccc(-c3c(Cl)cc4c(N5CC6CCC(C5)N6)nc(OCC5(CN6CCC(C#N)CC6)CC5)nc4c3F)c12 ; C#Cc1c(F)cc(F)c2cc(O)cc(-c3nc(OC)c4c(N5CC6CCC(C5)N6)nc(OCC5(CN6CC7CC7C6)CC5)nc4c3F)c12 ; C#Cc1c(F)cc(F)c2cc(O)cc(-c3nc(OC)c4c(N5CC6CCC(C5)N6)nc(OCC5(CN6CCC(C(F)(F)F)CC6)CC5)nc4c3F)c12]

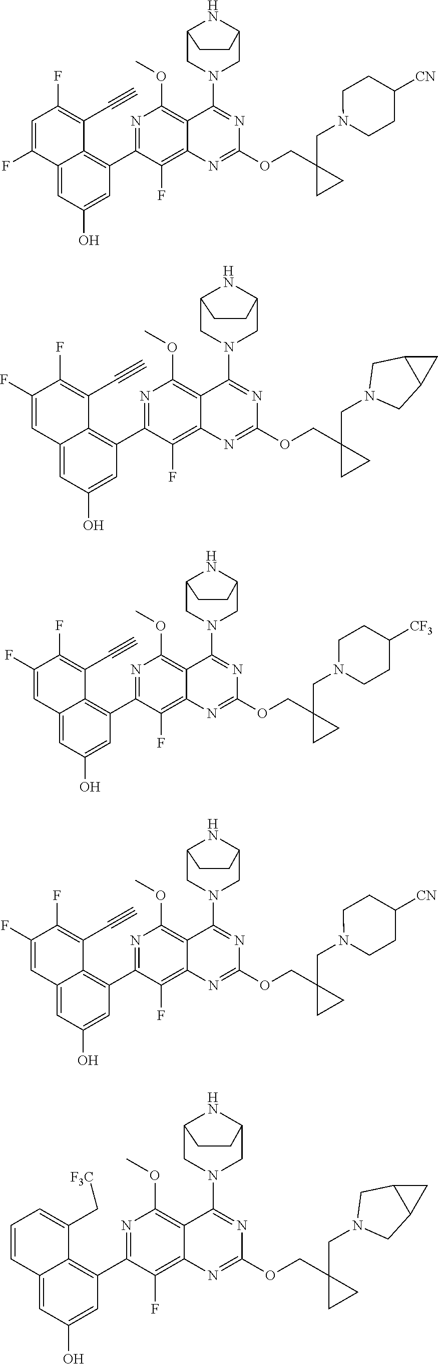 [SMILES: C#Cc1c(F)cc(F)c2cc(O)cc(-c3nc(OC)c4c(N5CC6CCC(C5)N6)nc(OCC5(CN6CCC(C#N)CC6)CC5)nc4c3F)c12 ; C#Cc1c(F)c(F)cc2cc(O)cc(-c3nc(OC)c4c(N5CC6CCC(C5)N6)nc(OCC5(CN6CC7CC7C6)CC5)nc4c3F)c12 ; C#Cc1c(F)c(F)cc2cc(O)cc(-c3nc(OC)c4c(N5CC6CCC(C5)N6)nc(OCC5(CN6CCC(C(F)(F)F)CC6)CC5)nc4c3F)c12 ; C#Cc1c(F)c(F)cc2cc(O)cc(-c3nc(OC)c4c(N5CC6CCC(C5)N6)nc(OCC5(CN6CCC(C#N)CC6)CC5)nc4c3F)c12 ; COc1nc(-c2cc(O)cc3cccc(CC(F)(F)F)c23)c(F)c2nc(OCC3(CN4CC5CC5C4)CC3)nc(N3CC4CCC(C3)N4)c12]

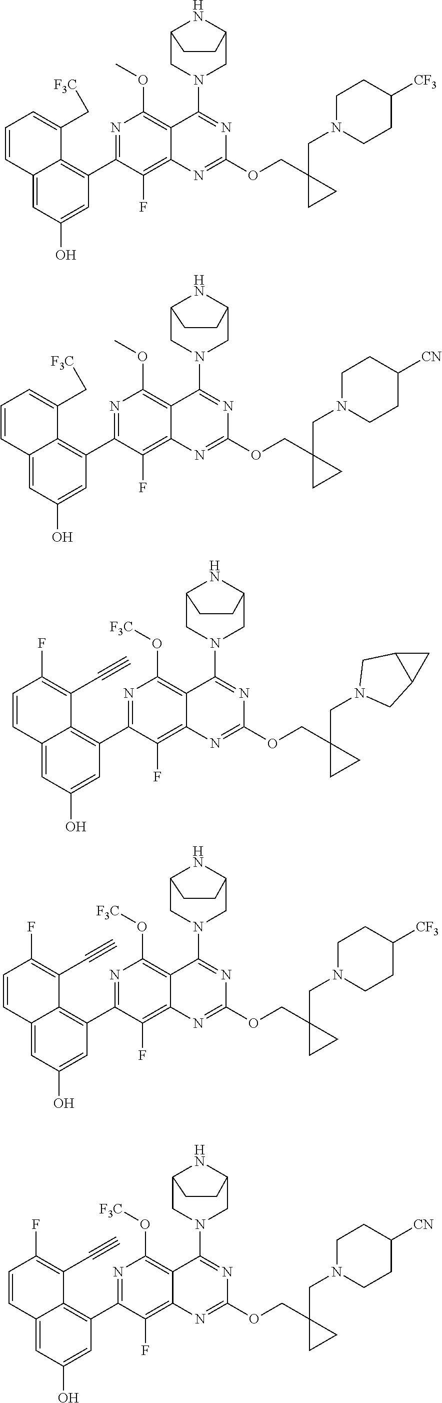 [SMILES: COc1nc(-c2cc(O)cc3cccc(CC(F)(F)F)c23)c(F)c2nc(OCC3(CN4CCC(C(F)(F)F)CC4)CC3)nc(N3CC4CCC(C3)N4)c12 ; COc1nc(-c2cc(O)cc3cccc(CC(F)(F)F)c23)c(F)c2nc(OCC3(CN4CCC(C#N)CC4)CC3)nc(N3CC4CCC(C3)N4)c12 ; C#Cc1c(F)ccc2cc(O)cc(-c3nc(OC(F)(F)F)c4c(N5CC6CCC(C5)N6)nc(OCC5(CN6CC7CC7C6)CC5)nc4c3F)c12 ; C#Cc1c(F)ccc2cc(O)cc(-c3nc(OC(F)(F)F)c4c(N5CC6CCC(C5)N6)nc(OCC5(CN6CCC(C(F)(F)F)CC6)CC5)nc4c3F)c12 ; C#Cc1c(F)ccc2cc(O)cc(-c3nc(OC(F)(F)F)c4c(N5CC6CCC(C5)N6)nc(OCC5(CN6CCC(C#N)CC6)CC5)nc4c3F)c12]

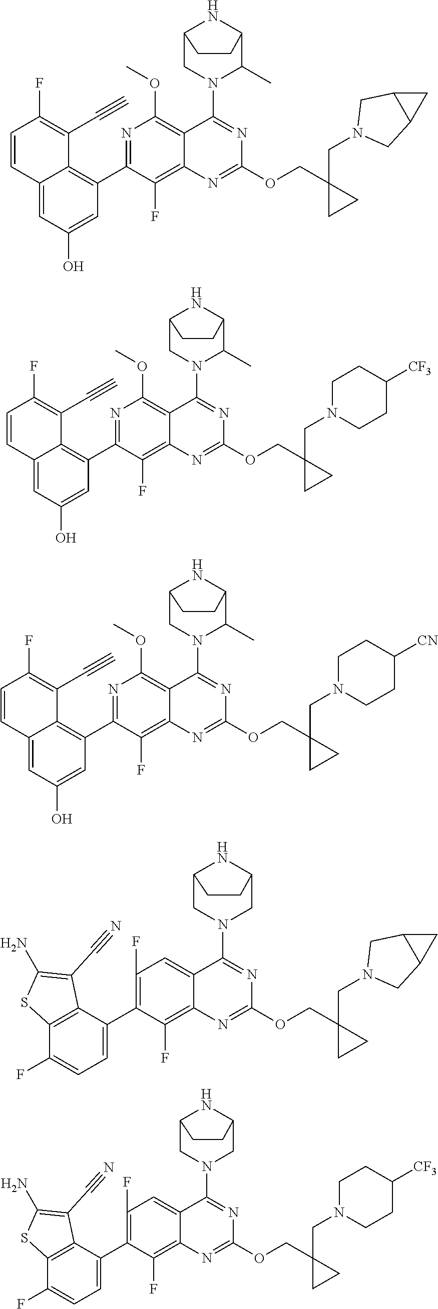 [SMILES: C#Cc1c(F)ccc2cc(O)cc(-c3nc(OC)c4c(N5CC6CCC(N6)C5C)nc(OCC5(CN6CC7CC7C6)CC5)nc4c3F)c12 ; C#Cc1c(F)ccc2cc(O)cc(-c3nc(OC)c4c(N5CC6CCC(N6)C5C)nc(OCC5(CN6CCC(C(F)(F)F)CC6)CC5)nc4c3F)c12 ; C#Cc1c(F)ccc2cc(O)cc(-c3nc(OC)c4c(N5CC6CCC(N6)C5C)nc(OCC5(CN6CCC(C#N)CC6)CC5)nc4c3F)c12 ; N#Cc1c(N)sc2c(F)ccc(-c3c(F)cc4c(N5CC6CCC(C5)N6)nc(OCC5(CN6CC7CC7C6)CC5)nc4c3F)c12 ; N#Cc1c(N)sc2c(F)ccc(-c3c(F)cc4c(N5CC6CCC(C5)N6)nc(OCC5(CN6CCC(C(F)(F)F)CC6)CC5)nc4c3F)c12]

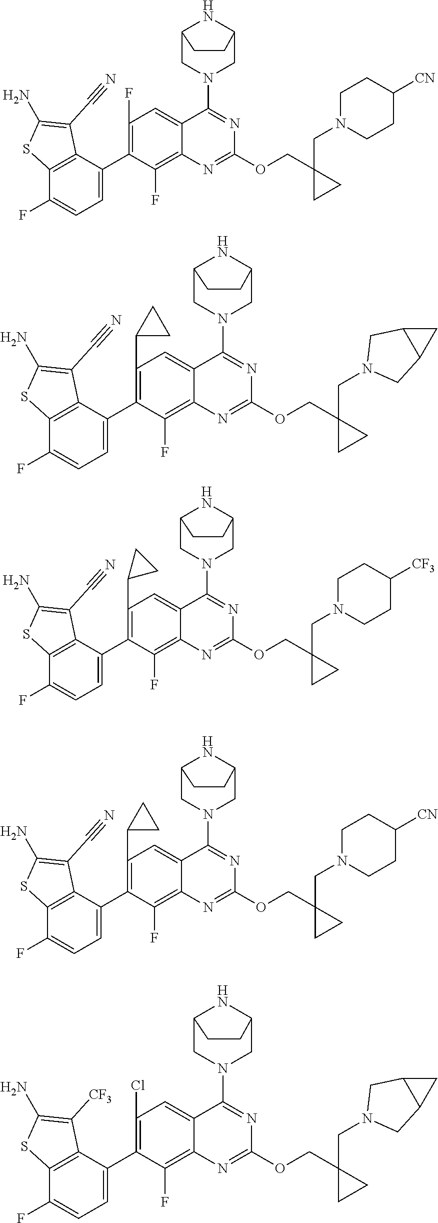 [SMILES: N#Cc1c(N)sc2c(F)ccc(-c3c(F)cc4c(N5CC6CCC(C5)N6)nc(OCC5(CN6CCC(C#N)CC6)CC5)nc4c3F)c12 ; N#Cc1c(N)sc2c(F)ccc(-c3c(C4CC4)cc4c(N5CC6CCC(C5)N6)nc(OCC5(CN6CC7CC7C6)CC5)nc4c3F)c12 ; N#Cc1c(N)sc2c(F)ccc(-c3c(C4CC4)cc4c(N5CC6CCC(C5)N6)nc(OCC5(CN6CCC(C(F)(F)F)CC6)CC5)nc4c3F)c12 ; N#Cc1c(N)sc2c(F)ccc(-c3c(C4CC4)cc4c(N5CC6CCC(C5)N6)nc(OCC5(CN6CCC(C#N)CC6)CC5)nc4c3F)c12 ; Nc1sc2c(F)ccc(-c3c(Cl)cc4c(N5CC6CCC(C5)N6)nc(OCC5(CN6CC7CC7C6)CC5)nc4c3F)c2c1C(F)(F)F]

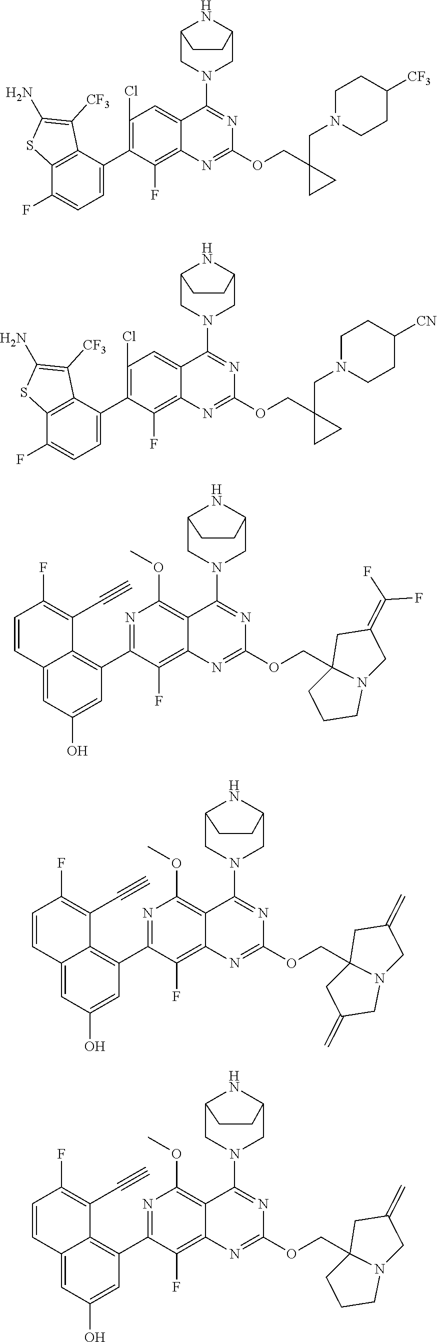 [SMILES: Nc1sc2c(F)ccc(-c3c(Cl)cc4c(N5CC6CCC(C5)N6)nc(OCC5(CN6CCC(C(F)(F)F)CC6)CC5)nc4c3F)c2c1C(F)(F)F ; N#CC1CCN(CC2(COc3nc(N4CC5CCC(C4)N5)c4cc(Cl)c(-c5ccc(F)c6sc(N)c(C(F)(F)F)c56)c(F)c4n3)CC2)CC1 ; C#Cc1c(F)ccc2cc(O)cc(-c3nc(OC)c4c(N5CC6CCC(C5)N6)nc(OCC56CCCN5CC(=C(F)F)C6)nc4c3F)c12 ; C#Cc1c(F)ccc2cc(O)cc(-c3nc(OC)c4c(N5CC6CCC(C5)N6)nc(OCC56CC(=C)CN5CC(=C)C6)nc4c3F)c12 ; C#Cc1c(F)ccc2cc(O)cc(-c3nc(OC)c4c(N5CC6CCC(C5)N6)nc(OCC56CCCN5CC(=C)C6)nc4c3F)c12]

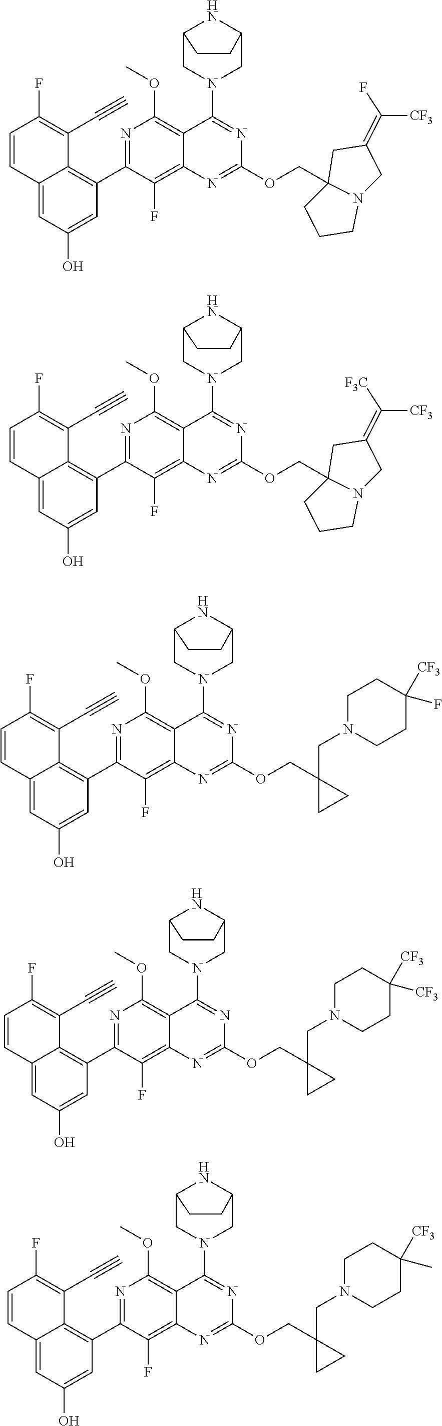 [SMILES: C#Cc1c(F)ccc2cc(O)cc(-c3nc(OC)c4c(N5CC6CCC(C5)N6)nc(OCC56CCCN5C/C(=C(/F)C(F)(F)F)C6)nc4c3F)c12 ; C#Cc1c(F)ccc2cc(O)cc(-c3nc(OC)c4c(N5CC6CCC(C5)N6)nc(OCC56CCCN5CC(=C(C(F)(F)F)C(F)(F)F)C6)nc4c3F)c12 ; C#Cc1c(F)ccc2cc(O)cc(-c3nc(OC)c4c(N5CC6CCC(C5)N6)nc(OCC5(CN6CCC(F)(C(F)(F)F)CC6)CC5)nc4c3F)c12 ; C#Cc1c(F)ccc2cc(O)cc(-c3nc(OC)c4c(N5CC6CCC(C5)N6)nc(OCC5(CN6CCC(C(F)(F)F)(C(F)(F)F)CC6)CC5)nc4c3F)c12 ; C#Cc1c(F)ccc2cc(O)cc(-c3nc(OC)c4c(N5CC6CCC(C5)N6)nc(OCC5(CN6CCC(C)(C(F)(F)F)CC6)CC5)nc4c3F)c12]

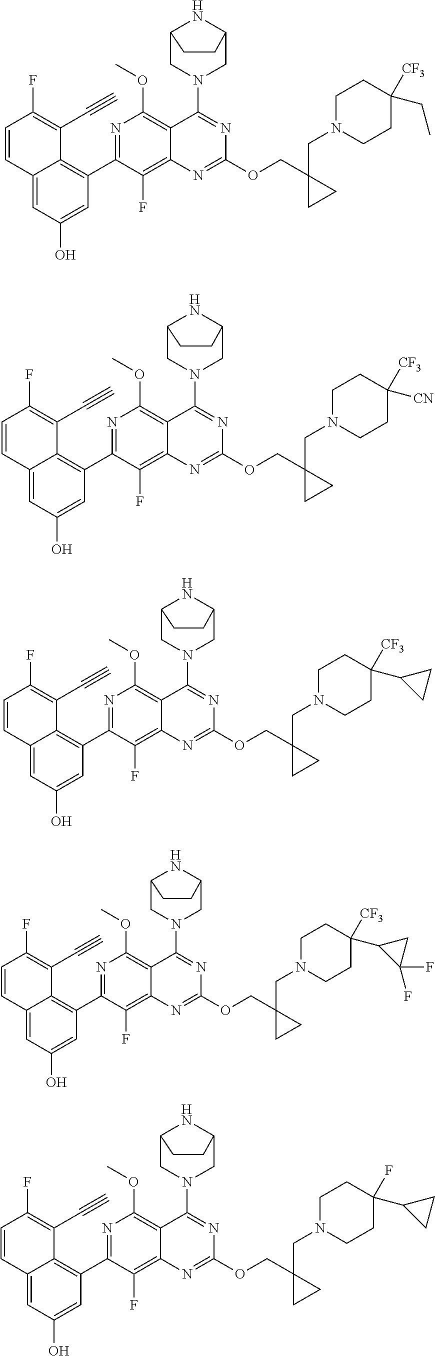 [SMILES: C#Cc1c(F)ccc2cc(O)cc(-c3nc(OC)c4c(N5CC6CCC(C5)N6)nc(OCC5(CN6CCC(CC)(C(F)(F)F)CC6)CC5)nc4c3F)c12 ; C#Cc1c(F)ccc2cc(O)cc(-c3nc(OC)c4c(N5CC6CCC(C5)N6)nc(OCC5(CN6CCC(C#N)(C(F)(F)F)CC6)CC5)nc4c3F)c12 ; C#Cc1c(F)ccc2cc(O)cc(-c3nc(OC)c4c(N5CC6CCC(C5)N6)nc(OCC5(CN6CCC(C7CC7)(C(F)(F)F)CC6)CC5)nc4c3F)c12 ; C#Cc1c(F)ccc2cc(O)cc(-c3nc(OC)c4c(N5CC6CCC(C5)N6)nc(OCC5(CN6CCC(C7CC7(F)F)(C(F)(F)F)CC6)CC5)nc4c3F)c12 ; C#Cc1c(F)ccc2cc(O)cc(-c3nc(OC)c4c(N5CC6CCC(C5)N6)nc(OCC5(CN6CCC(F)(C7CC7)CC6)CC5)nc4c3F)c12]

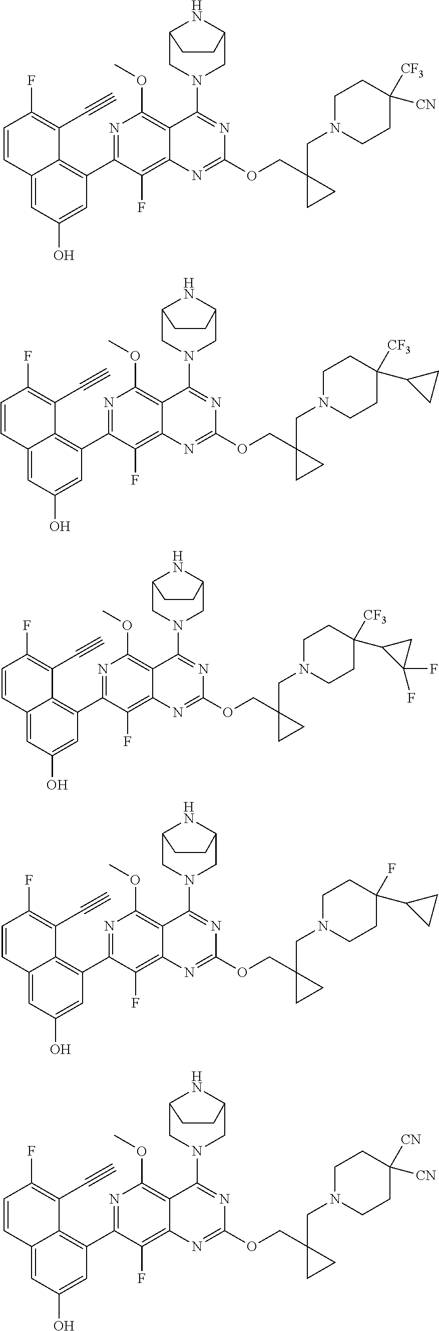 [SMILES: C#Cc1c(F)ccc2cc(O)cc(-c3nc(OC)c4c(N5CC6CCC(C5)N6)nc(OCC5(CN6CCC(C#N)(C(F)(F)F)CC6)CC5)nc4c3F)c12 ; C#Cc1c(F)ccc2cc(O)cc(-c3nc(OC)c4c(N5CC6CCC(C5)N6)nc(OCC5(CN6CCC(C7CC7)(C(F)(F)F)CC6)CC5)nc4c3F)c12 ; C#Cc1c(F)ccc2cc(O)cc(-c3nc(OC)c4c(N5CC6CCC(C5)N6)nc(OCC5(CN6CCC(C7CC7(F)F)(C(F)(F)F)CC6)CC5)nc4c3F)c12 ; C#Cc1c(F)ccc2cc(O)cc(-c3nc(OC)c4c(N5CC6CCC(C5)N6)nc(OCC5(CN6CCC(F)(C7CC7)CC6)CC5)nc4c3F)c12 ; C#Cc1c(F)ccc2cc(O)cc(-c3nc(OC)c4c(N5CC6CCC(C5)N6)nc(OCC5(CN6CCC(C#N)(C#N)CC6)CC5)nc4c3F)c12]

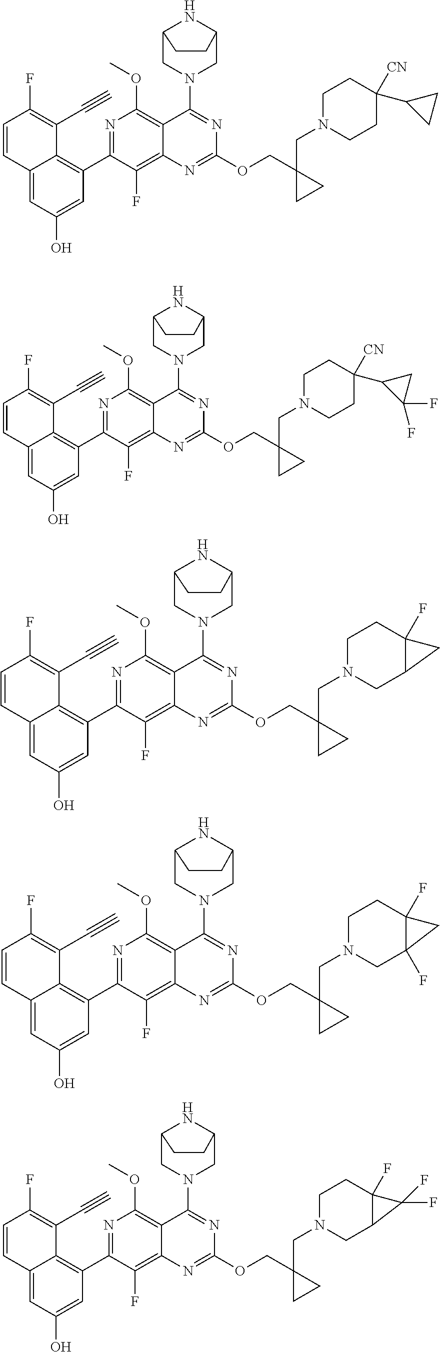 [SMILES: C#Cc1c(F)ccc2cc(O)cc(-c3nc(OC)c4c(N5CC6CCC(C5)N6)nc(OCC5(CN6CCC(C#N)(C7CC7)CC6)CC5)nc4c3F)c12 ; C#Cc1c(F)ccc2cc(O)cc(-c3nc(OC)c4c(N5CC6CCC(C5)N6)nc(OCC5(CN6CCC(C#N)(C7CC7(F)F)CC6)CC5)nc4c3F)c12 ; C#Cc1c(F)ccc2cc(O)cc(-c3nc(OC)c4c(N5CC6CCC(C5)N6)nc(OCC5(CN6CCC7(F)CC7C6)CC5)nc4c3F)c12 ; C#Cc1c(F)ccc2cc(O)cc(-c3nc(OC)c4c(N5CC6CCC(C5)N6)nc(OCC5(CN6CCC7(F)CC7(F)C6)CC5)nc4c3F)c12 ; C#Cc1c(F)ccc2cc(O)cc(-c3nc(OC)c4c(N5CC6CCC(C5)N6)nc(OCC5(CN6CCC7(F)C(C6)C7(F)F)CC5)nc4c3F)c12]

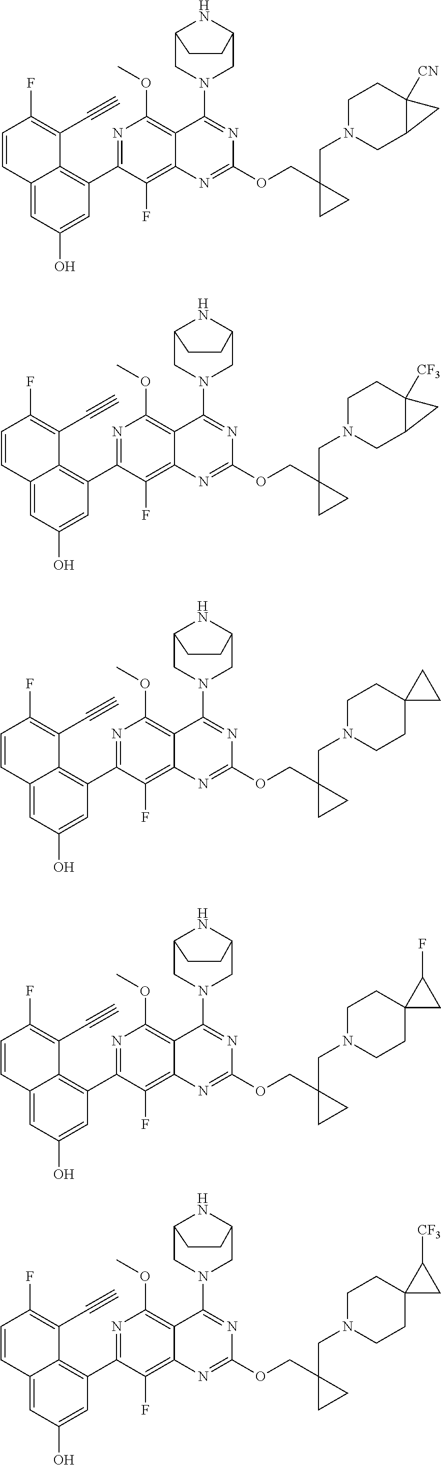 [SMILES: C#Cc1c(F)ccc2cc(O)cc(-c3nc(OC)c4c(N5CC6CCC(C5)N6)nc(OCC5(CN6CCC7(C#N)CC7C6)CC5)nc4c3F)c12 ; C#Cc1c(F)ccc2cc(O)cc(-c3nc(OC)c4c(N5CC6CCC(C5)N6)nc(OCC5(CN6CCC7(C(F)(F)F)CC7C6)CC5)nc4c3F)c12 ; C#Cc1c(F)ccc2cc(O)cc(-c3nc(OC)c4c(N5CC6CCC(C5)N6)nc(OCC5(CN6CCC7(CC6)CC7)CC5)nc4c3F)c12 ; C#Cc1c(F)ccc2cc(O)cc(-c3nc(OC)c4c(N5CC6CCC(C5)N6)nc(OCC5(CN6CCC7(CC6)CC7F)CC5)nc4c3F)c12 ; C#Cc1c(F)ccc2cc(O)cc(-c3nc(OC)c4c(N5CC6CCC(C5)N6)nc(OCC5(CN6CCC7(CC6)CC7C(F)(F)F)CC5)nc4c3F)c12]

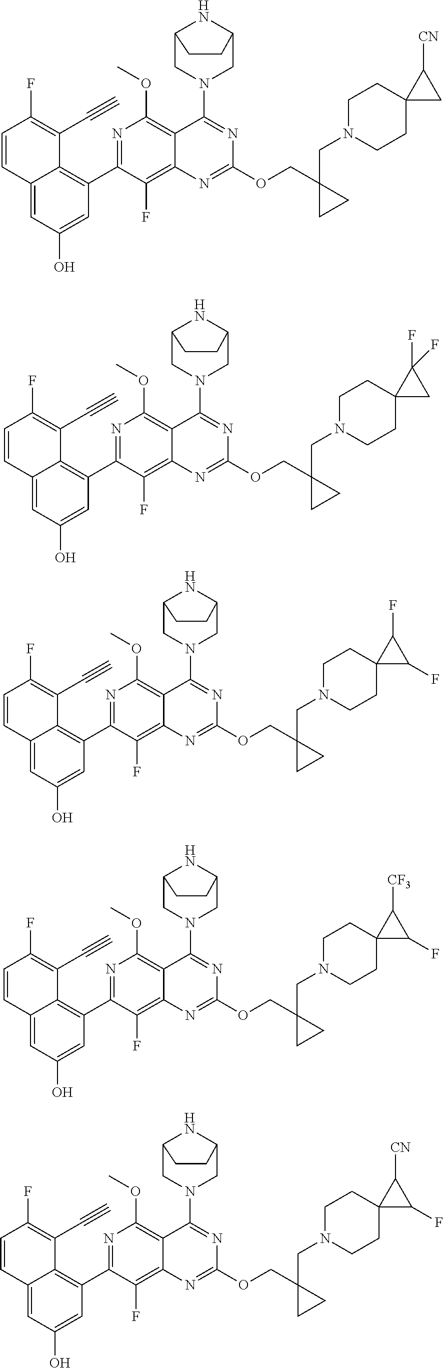 [SMILES: C#Cc1c(F)ccc2cc(O)cc(-c3nc(OC)c4c(N5CC6CCC(C5)N6)nc(OCC5(CN6CCC7(CC6)CC7C#N)CC5)nc4c3F)c12 ; C#Cc1c(F)ccc2cc(O)cc(-c3nc(OC)c4c(N5CC6CCC(C5)N6)nc(OCC5(CN6CCC7(CC6)CC7(F)F)CC5)nc4c3F)c12 ; C#Cc1c(F)ccc2cc(O)cc(-c3nc(OC)c4c(N5CC6CCC(C5)N6)nc(OCC5(CN6CCC7(CC6)C(F)C7F)CC5)nc4c3F)c12 ; C#Cc1c(F)ccc2cc(O)cc(-c3nc(OC)c4c(N5CC6CCC(C5)N6)nc(OCC5(CN6CCC7(CC6)C(F)C7C(F)(F)F)CC5)nc4c3F)c12 ; C#Cc1c(F)ccc2cc(O)cc(-c3nc(OC)c4c(N5CC6CCC(C5)N6)nc(OCC5(CN6CCC7(CC6)C(F)C7C#N)CC5)nc4c3F)c12]

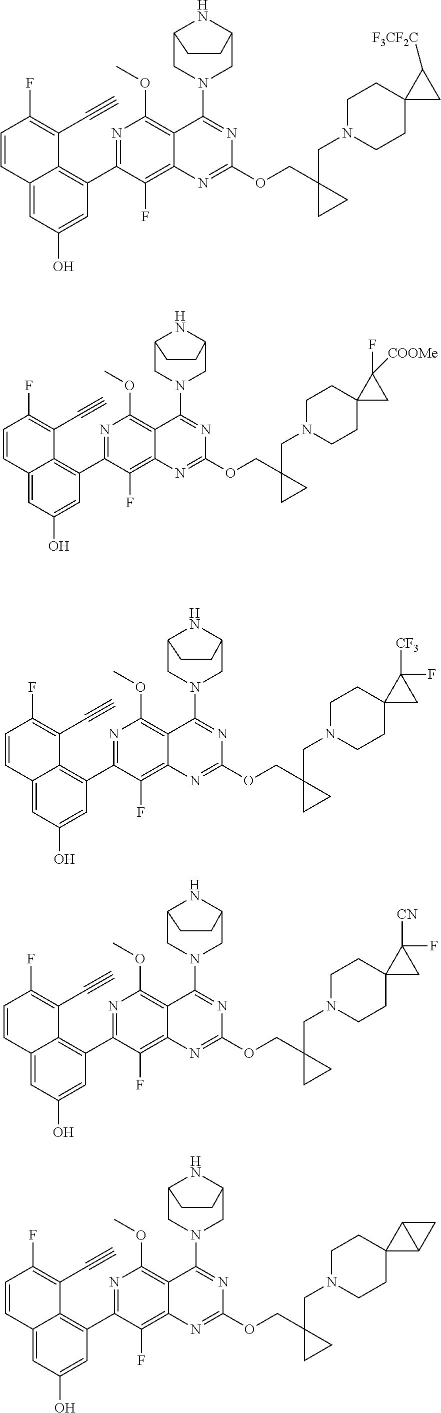

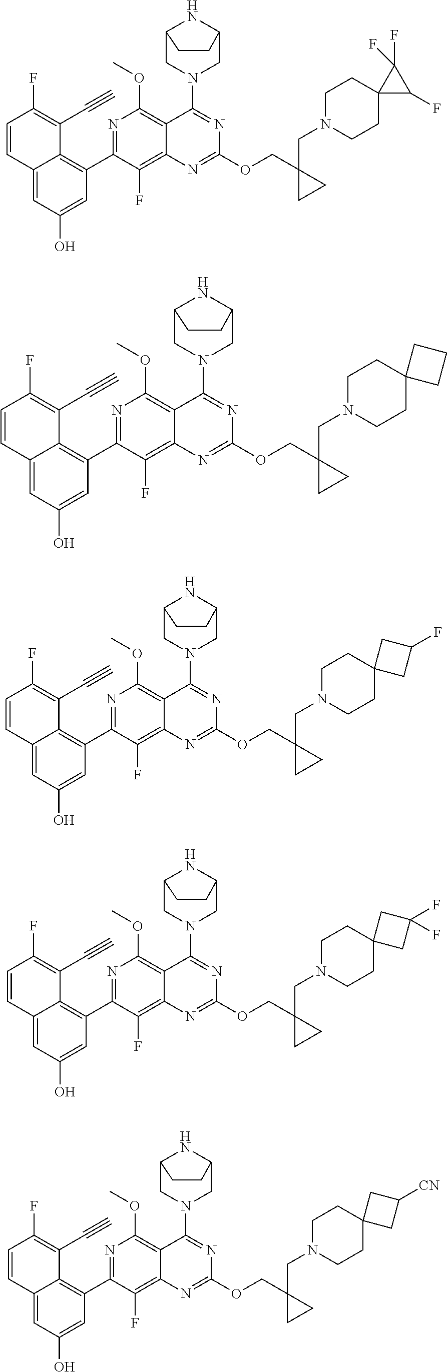 [SMILES: C#Cc1c(F)ccc2cc(O)cc(-c3nc(OC)c4c(N5CC6CCC(C5)N6)nc(OCC5(CN6CCC7(CC6)C(F)C7(F)F)CC5)nc4c3F)c12 ; C#Cc1c(F)ccc2cc(O)cc(-c3nc(OC)c4c(N5CC6CCC(C5)N6)nc(OCC5(CN6CCC7(CCC7)CC6)CC5)nc4c3F)c12 ; C#Cc1c(F)ccc2cc(O)cc(-c3nc(OC)c4c(N5CC6CCC(C5)N6)nc(OCC5(CN6CCC7(CC6)CC(F)C7)CC5)nc4c3F)c12 ; C#Cc1c(F)ccc2cc(O)cc(-c3nc(OC)c4c(N5CC6CCC(C5)N6)nc(OCC5(CN6CCC7(CC6)CC(F)(F)C7)CC5)nc4c3F)c12 ; C#Cc1c(F)ccc2cc(O)cc(-c3nc(OC)c4c(N5CC6CCC(C5)N6)nc(OCC5(CN6CCC7(CC6)CC(C#N)C7)CC5)nc4c3F)c12]

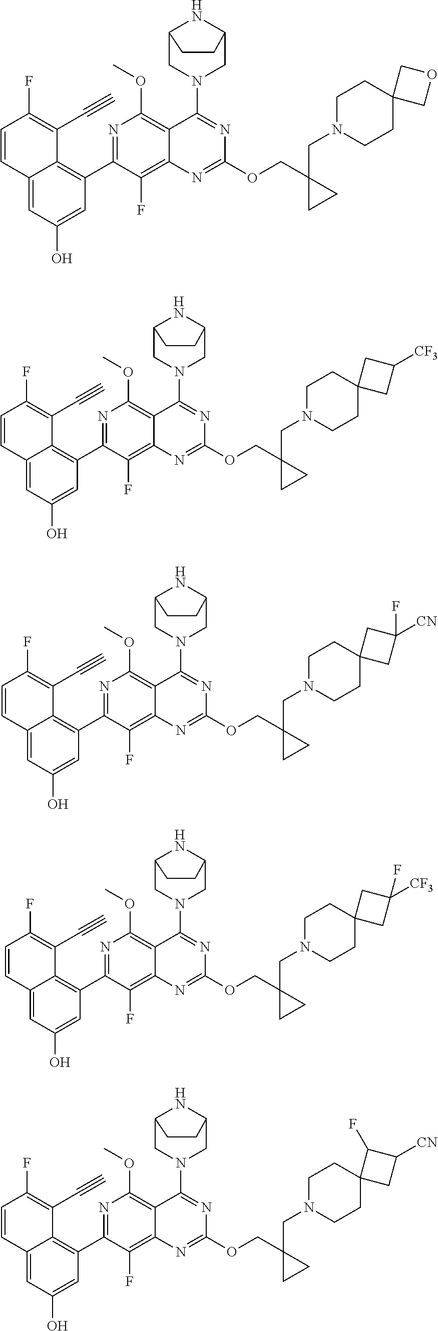 [SMILES: C#Cc1c(F)ccc2cc(O)cc(-c3nc(OC)c4c(N5CC6CCC(C5)N6)nc(OCC5(CN6CCC7(CC6)COC7)CC5)nc4c3F)c12 ; C#Cc1c(F)ccc2cc(O)cc(-c3nc(OC)c4c(N5CC6CCC(C5)N6)nc(OCC5(CN6CCC7(CC6)CC(C(F)(F)F)C7)CC5)nc4c3F)c12 ; C#Cc1c(F)ccc2cc(O)cc(-c3nc(OC)c4c(N5CC6CCC(C5)N6)nc(OCC5(CN6CCC7(CC6)CC(F)(C#N)C7)CC5)nc4c3F)c12 ; C#Cc1c(F)ccc2cc(O)cc(-c3nc(OC)c4c(N5CC6CCC(C5)N6)nc(OCC5(CN6CCC7(CC6)CC(F)(C(F)(F)F)C7)CC5)nc4c3F)c12 ; C#Cc1c(F)ccc2cc(O)cc(-c3nc(OC)c4c(N5CC6CCC(C5)N6)nc(OCC5(CN6CCC7(CC6)CC(C#N)C7F)CC5)nc4c3F)c12]

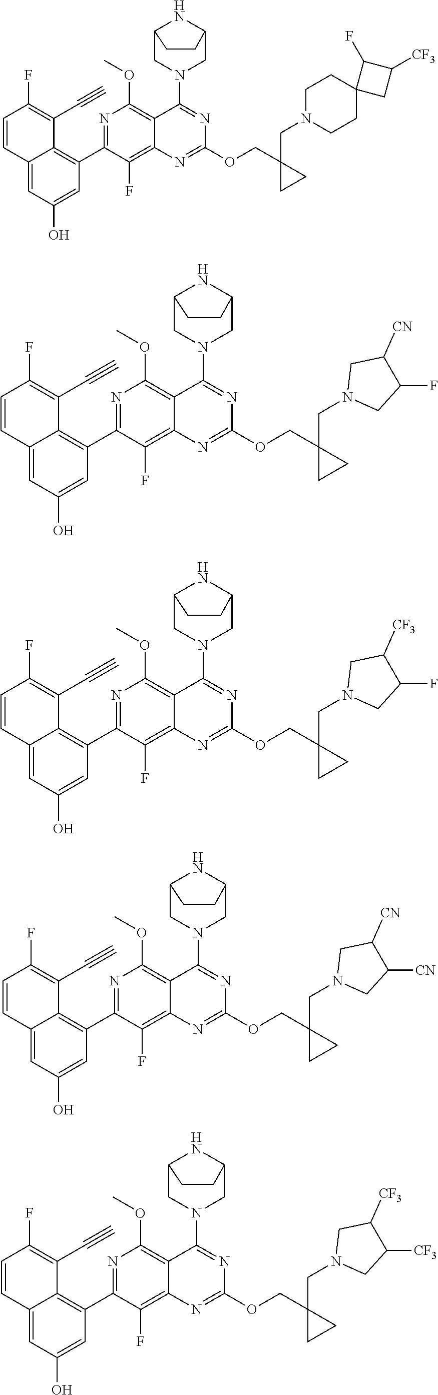 [SMILES: C#Cc1c(F)ccc2cc(O)cc(-c3nc(OC)c4c(N5CC6CCC(C5)N6)nc(OCC5(CN6CCC7(CC6)CC(C(F)(F)F)C7F)CC5)nc4c3F)c12 ; C#Cc1c(F)ccc2cc(O)cc(-c3nc(OC)c4c(N5CC6CCC(C5)N6)nc(OCC5(CN6CC(F)C(C#N)C6)CC5)nc4c3F)c12 ; C#Cc1c(F)ccc2cc(O)cc(-c3nc(OC)c4c(N5CC6CCC(C5)N6)nc(OCC5(CN6CC(F)C(C(F)(F)F)C6)CC5)nc4c3F)c12 ; C#Cc1c(F)ccc2cc(O)cc(-c3nc(OC)c4c(N5CC6CCC(C5)N6)nc(OCC5(CN6CC(C#N)C(C#N)C6)CC5)nc4c3F)c12 ; C#Cc1c(F)ccc2cc(O)cc(-c3nc(OC)c4c(N5CC6CCC(C5)N6)nc(OCC5(CN6CC(C(F)(F)F)C(C(F)(F)F)C6)CC5)nc4c3F)c12]

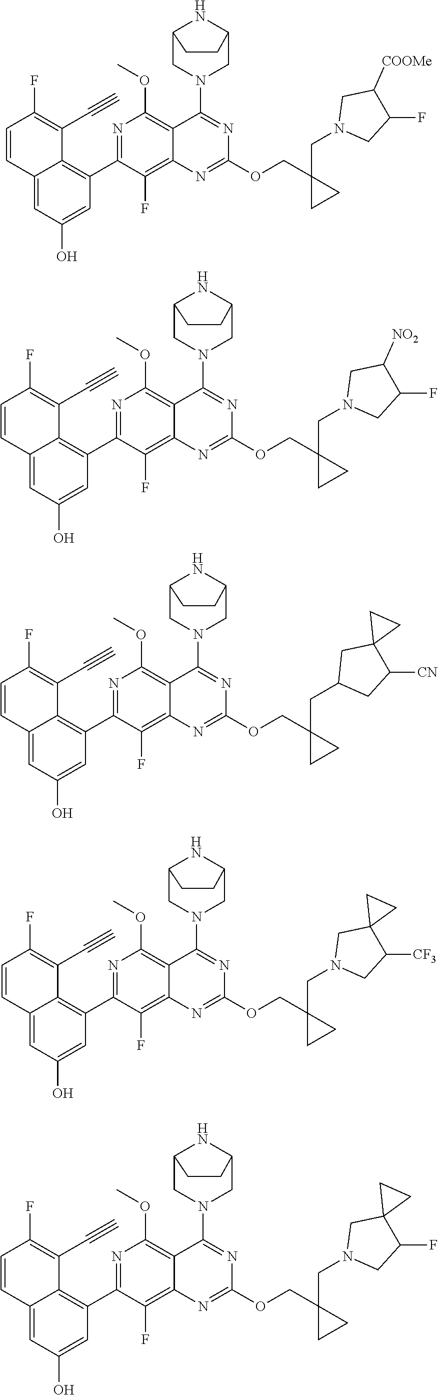 [SMILES: C#Cc1c(F)ccc2cc(O)cc(-c3nc(OC)c4c(N5CC6CCC(C5)N6)nc(OCC5(CN6CC(F)C(C(=O)OC)C6)CC5)nc4c3F)c12 ; C#Cc1c(F)ccc2cc(O)cc(-c3nc(OC)c4c(N5CC6CCC(C5)N6)nc(OCC5(CN6CC(F)C([N+](=O)[O-])C6)CC5)nc4c3F)c12 ; C#Cc1c(F)ccc2cc(O)cc(-c3nc(OC)c4c(N5CC6CCC(C5)N6)nc(OCC5(CC6CC(C#N)C7(CC7)C6)CC5)nc4c3F)c12 ; C#Cc1c(F)ccc2cc(O)cc(-c3nc(OC)c4c(N5CC6CCC(C5)N6)nc(OCC5(CN6CC(C(F)(F)F)C7(CC7)C6)CC5)nc4c3F)c12 ; C#Cc1c(F)ccc2cc(O)cc(-c3nc(OC)c4c(N5CC6CCC(C5)N6)nc(OCC5(CN6CC(F)C7(CC7)C6)CC5)nc4c3F)c12]

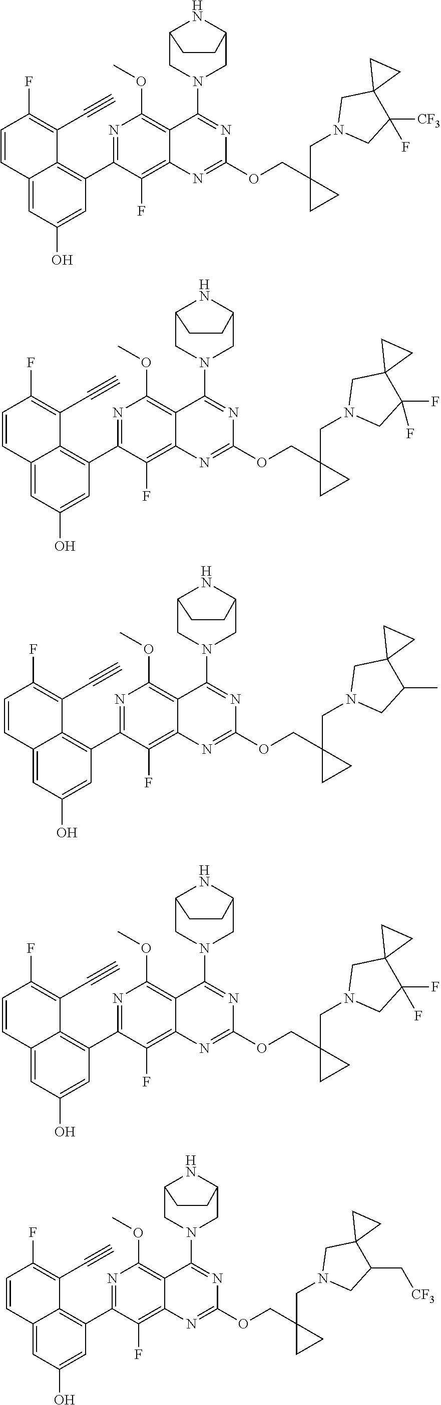 [SMILES: C#Cc1c(F)ccc2cc(O)cc(-c3nc(OC)c4c(N5CC6CCC(C5)N6)nc(OCC5(CN6CC7(CC7)C(F)(C(F)(F)F)C6)CC5)nc4c3F)c12 ; C#Cc1c(F)ccc2cc(O)cc(-c3nc(OC)c4c(N5CC6CCC(C5)N6)nc(OCC5(CN6CC(F)(F)C7(CC7)C6)CC5)nc4c3F)c12 ; C#Cc1c(F)ccc2cc(O)cc(-c3nc(OC)c4c(N5CC6CCC(C5)N6)nc(OCC5(CN6CC(C)C7(CC7)C6)CC5)nc4c3F)c12 ; C#Cc1c(F)ccc2cc(O)cc(-c3nc(OC)c4c(N5CC6CCC(C5)N6)nc(OCC5(CN6CC(F)(F)C7(CC7)C6)CC5)nc4c3F)c12 ; C#Cc1c(F)ccc2cc(O)cc(-c3nc(OC)c4c(N5CC6CCC(C5)N6)nc(OCC5(CN6CC(CC(F)(F)F)C7(CC7)C6)CC5)nc4c3F)c12]

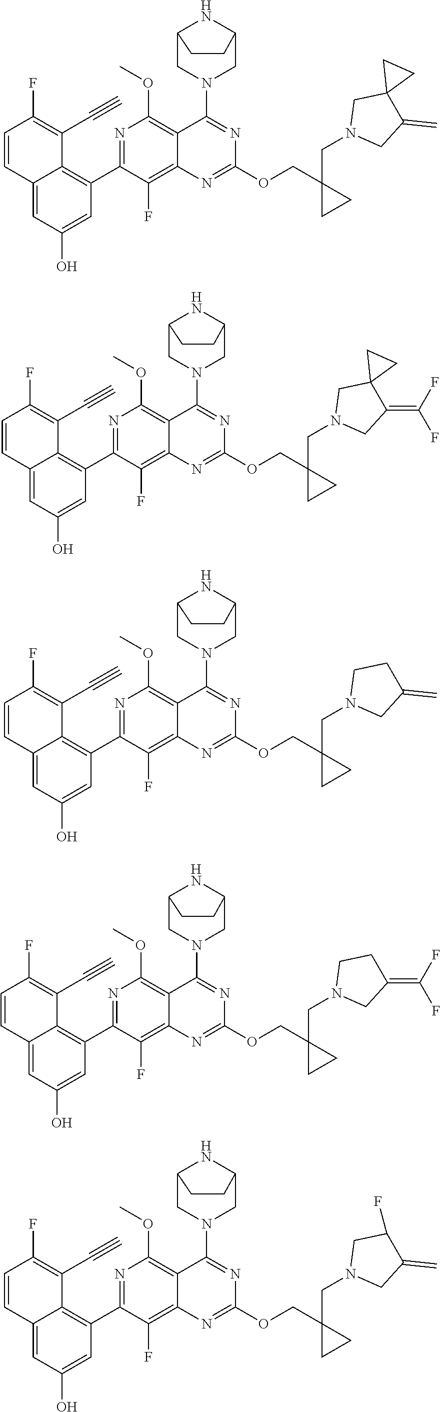 [SMILES: C#Cc1c(F)ccc2cc(O)cc(-c3nc(OC)c4c(N5CC6CCC(C5)N6)nc(OCC5(CN6CC(=C)C7(CC7)C6)CC5)nc4c3F)c12 ; C#Cc1c(F)ccc2cc(O)cc(-c3nc(OC)c4c(N5CC6CCC(C5)N6)nc(OCC5(CN6CC(=C(F)F)C7(CC7)C6)CC5)nc4c3F)c12 ; C#Cc1c(F)ccc2cc(O)cc(-c3nc(OC)c4c(N5CC6CCC(C5)N6)nc(OCC5(CN6CCC(=C)C6)CC5)nc4c3F)c12 ; C#Cc1c(F)ccc2cc(O)cc(-c3nc(OC)c4c(N5CC6CCC(C5)N6)nc(OCC5(CN6CCC(=C(F)F)C6)CC5)nc4c3F)c12 ; C#Cc1c(F)ccc2cc(O)cc(-c3nc(OC)c4c(N5CC6CCC(C5)N6)nc(OCC5(CN6CC(=C)C(F)C6)CC5)nc4c3F)c12]

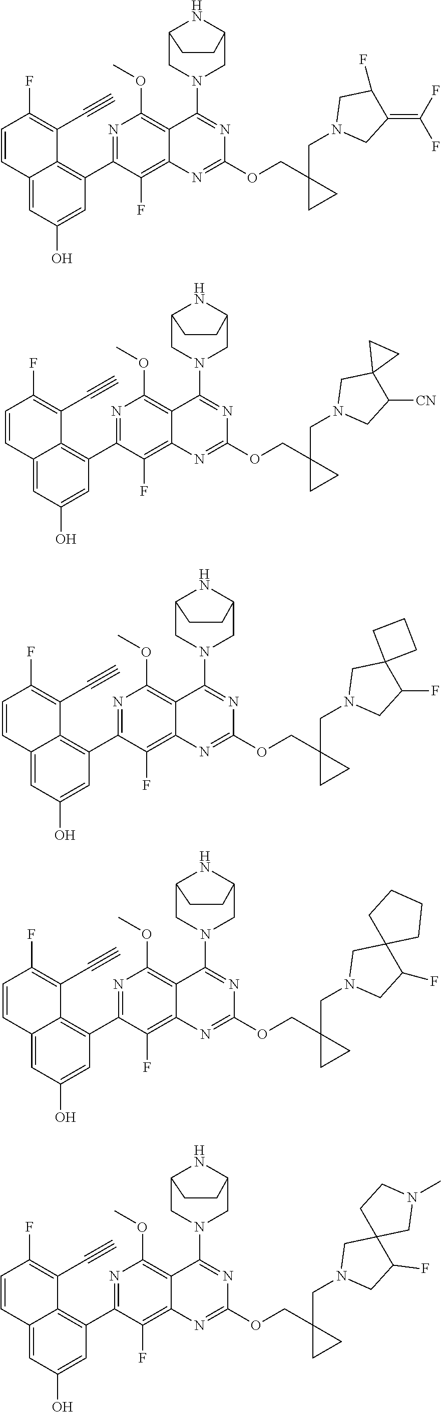 [SMILES: C#Cc1c(F)ccc2cc(O)cc(-c3nc(OC)c4c(N5CC6CCC(C5)N6)nc(OCC5(CN6CC(=C(F)F)C(F)C6)CC5)nc4c3F)c12 ; C#Cc1c(F)ccc2cc(O)cc(-c3nc(OC)c4c(N5CC6CCC(C5)N6)nc(OCC5(CN6CC(C#N)C7(CC7)C6)CC5)nc4c3F)c12 ; C#Cc1c(F)ccc2cc(O)cc(-c3nc(OC)c4c(N5CC6CCC(C5)N6)nc(OCC5(CN6CC(F)C7(CCC7)C6)CC5)nc4c3F)c12 ; C#Cc1c(F)ccc2cc(O)cc(-c3nc(OC)c4c(N5CC6CCC(C5)N6)nc(OCC5(CN6CC(F)C7(CCCC7)C6)CC5)nc4c3F)c12 ; C#Cc1c(F)ccc2cc(O)cc(-c3nc(OC)c4c(N5CC6CCC(C5)N6)nc(OCC5(CN6CC(F)C7(CCN(C)C7)C6)CC5)nc4c3F)c12]

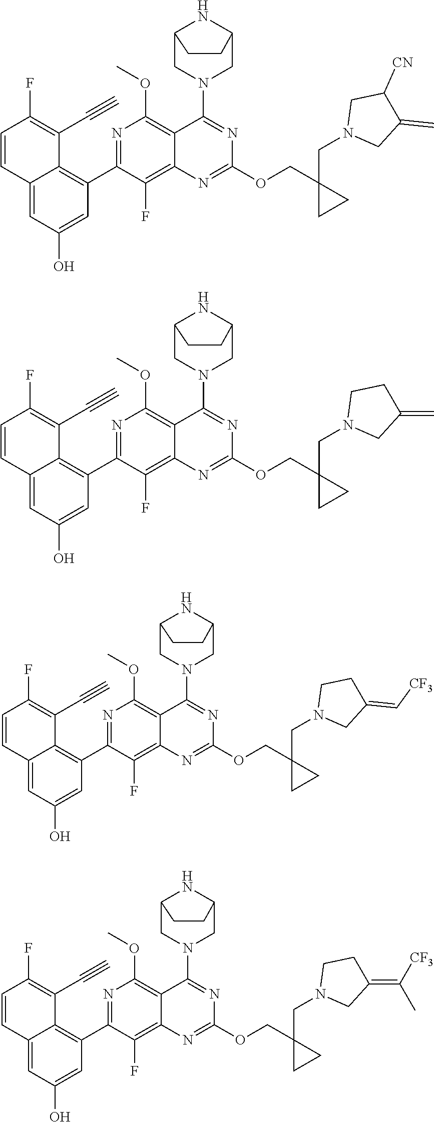 [SMILES: C#Cc1c(F)ccc2cc(O)cc(-c3nc(OC)c4c(N5CC6CCC(C5)N6)nc(OCC5(CN6CC(=C)C(C#N)C6)CC5)nc4c3F)c12 ; C#Cc1c(F)ccc2cc(O)cc(-c3nc(OC)c4c(N5CC6CCC(C5)N6)nc(OCC5(CN6CCC(=C)C6)CC5)nc4c3F)c12 ; C#Cc1c(F)ccc2cc(O)cc(-c3nc(OC)c4c(N5CC6CCC(C5)N6)nc(OCC5(CN6CC/C(=C\C(F)(F)F)C6)CC5)nc4c3F)c12 ; C#Cc1c(F)ccc2cc(O)cc(-c3nc(OC)c4c(N5CC6CCC(C5)N6)nc(OCC5(CN6CC/C(=C(/C)C(F)(F)F)C6)CC5)nc4c3F)c12]

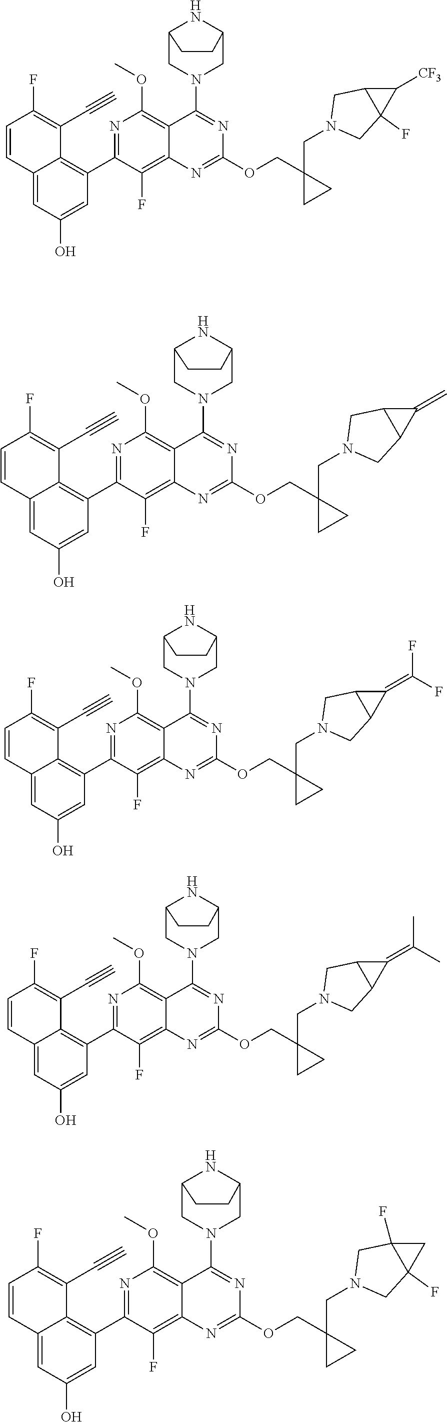 [SMILES: C#Cc1c(F)ccc2cc(O)cc(-c3nc(OC)c4c(N5CC6CCC(C5)N6)nc(OCC5(CN6CC7C(C(F)(F)F)C7(F)C6)CC5)nc4c3F)c12 ; C#Cc1c(F)ccc2cc(O)cc(-c3nc(OC)c4c(N5CC6CCC(C5)N6)nc(OCC5(CN6CC7C(=C)C7C6)CC5)nc4c3F)c12 ; C#Cc1c(F)ccc2cc(O)cc(-c3nc(OC)c4c(N5CC6CCC(C5)N6)nc(OCC5(CN6CC7C(=C(F)F)C7C6)CC5)nc4c3F)c12 ; C#Cc1c(F)ccc2cc(O)cc(-c3nc(OC)c4c(N5CC6CCC(C5)N6)nc(OCC5(CN6CC7C(=C(C)C)C7C6)CC5)nc4c3F)c12 ; C#Cc1c(F)ccc2cc(O)cc(-c3nc(OC)c4c(N5CC6CCC(C5)N6)nc(OCC5(CN6CC7(F)CC7(F)C6)CC5)nc4c3F)c12]

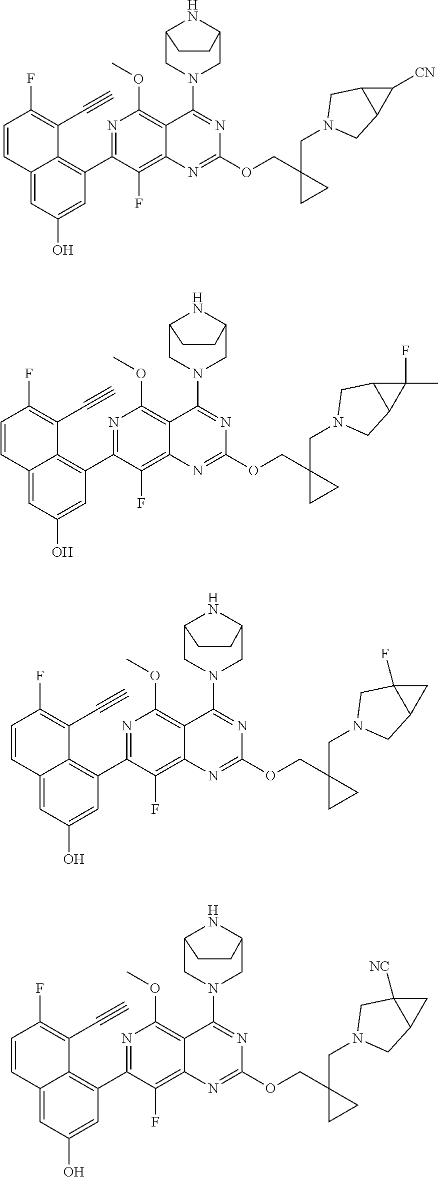 [SMILES: C#Cc1c(F)ccc2cc(O)cc(-c3nc(OC)c4c(N5CC6CCC(C5)N6)nc(OCC5(CN6CC7C(C#N)C7C6)CC5)nc4c3F)c12 ; C#Cc1c(F)ccc2cc(O)cc(-c3nc(OC)c4c(N5CC6CCC(C5)N6)nc(OCC5(CN6CC7C(C6)C7(C)F)CC5)nc4c3F)c12 ; C#Cc1c(F)ccc2cc(O)cc(-c3nc(OC)c4c(N5CC6CCC(C5)N6)nc(OCC5(CN6CC7CC7(F)C6)CC5)nc4c3F)c12 ; C#Cc1c(F)ccc2cc(O)cc(-c3nc(OC)c4c(N5CC6CCC(C5)N6)nc(OCC5(CN6CC7CC7(C#N)C6)CC5)nc4c3F)c12]

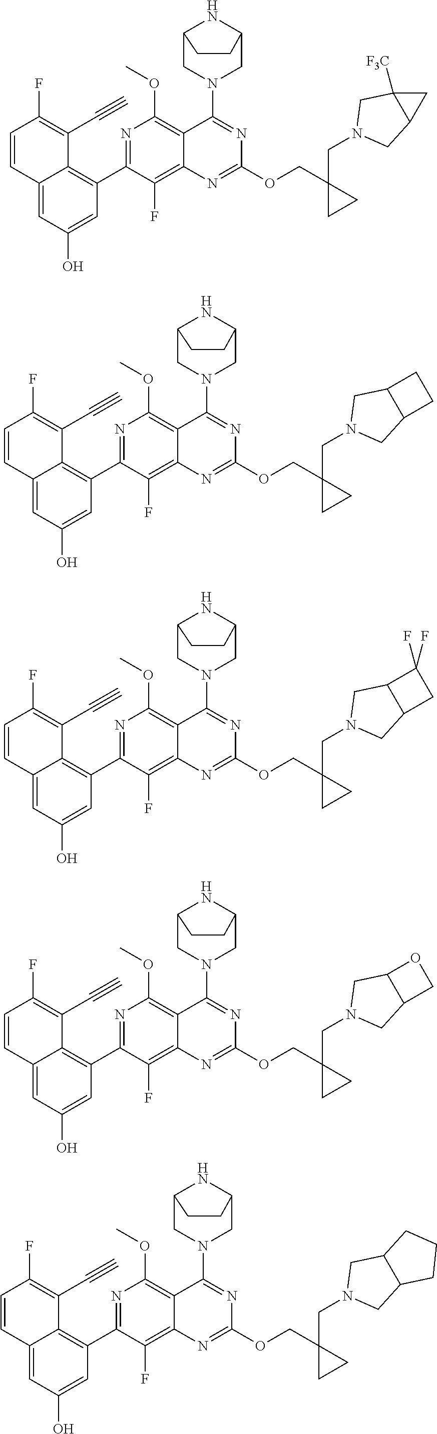 [SMILES: C#Cc1c(F)ccc2cc(O)cc(-c3nc(OC)c4c(N5CC6CCC(C5)N6)nc(OCC5(CN6CC7CC7(C(F)(F)F)C6)CC5)nc4c3F)c12 ; C#Cc1c(F)ccc2cc(O)cc(-c3nc(OC)c4c(N5CC6CCC(C5)N6)nc(OCC5(CN6CC7CCC7C6)CC5)nc4c3F)c12 ; C#Cc1c(F)ccc2cc(O)cc(-c3nc(OC)c4c(N5CC6CCC(C5)N6)nc(OCC5(CN6CC7CC(F)(F)C7C6)CC5)nc4c3F)c12 ; C#Cc1c(F)ccc2cc(O)cc(-c3nc(OC)c4c(N5CC6CCC(C5)N6)nc(OCC5(CN6CC7COC7C6)CC5)nc4c3F)c12 ; C#Cc1c(F)ccc2cc(O)cc(-c3nc(OC)c4c(N5CC6CCC(C5)N6)nc(OCC5(CN6CC7CCCC7C6)CC5)nc4c3F)c12]

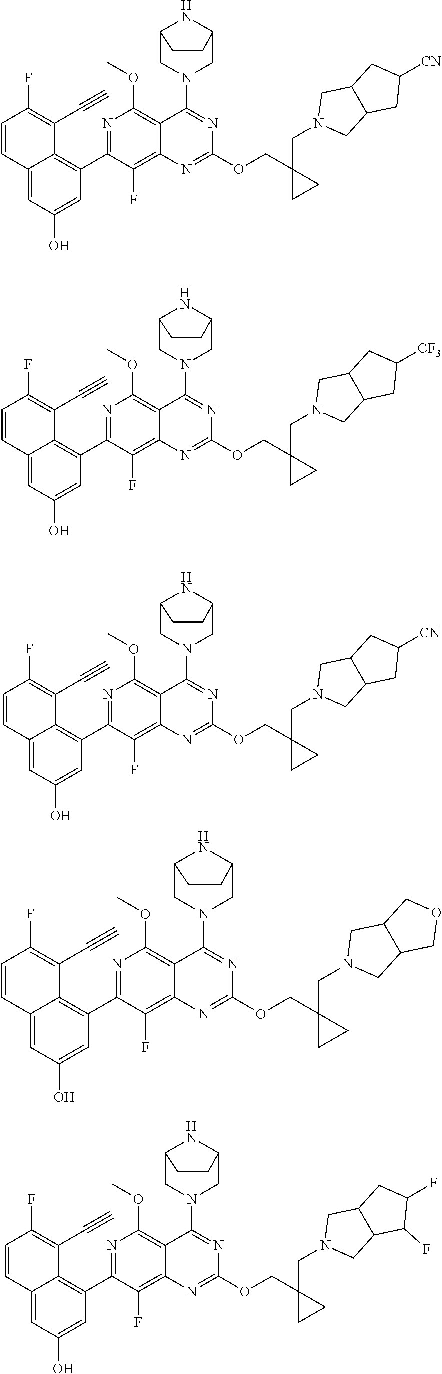 [SMILES: C#Cc1c(F)ccc2cc(O)cc(-c3nc(OC)c4c(N5CC6CCC(C5)N6)nc(OCC5(CN6CC7CC(C#N)CC7C6)CC5)nc4c3F)c12 ; C#Cc1c(F)ccc2cc(O)cc(-c3nc(OC)c4c(N5CC6CCC(C5)N6)nc(OCC5(CN6CC7CC(C(F)(F)F)CC7C6)CC5)nc4c3F)c12 ; C#Cc1c(F)ccc2cc(O)cc(-c3nc(OC)c4c(N5CC6CCC(C5)N6)nc(OCC5(CN6CC7CC(C#N)CC7C6)CC5)nc4c3F)c12 ; C#Cc1c(F)ccc2cc(O)cc(-c3nc(OC)c4c(N5CC6CCC(C5)N6)nc(OCC5(CN6CC7COCC7C6)CC5)nc4c3F)c12 ; C#Cc1c(F)ccc2cc(O)cc(-c3nc(OC)c4c(N5CC6CCC(C5)N6)nc(OCC5(CN6CC7CC(F)C(F)C7C6)CC5)nc4c3F)c12]

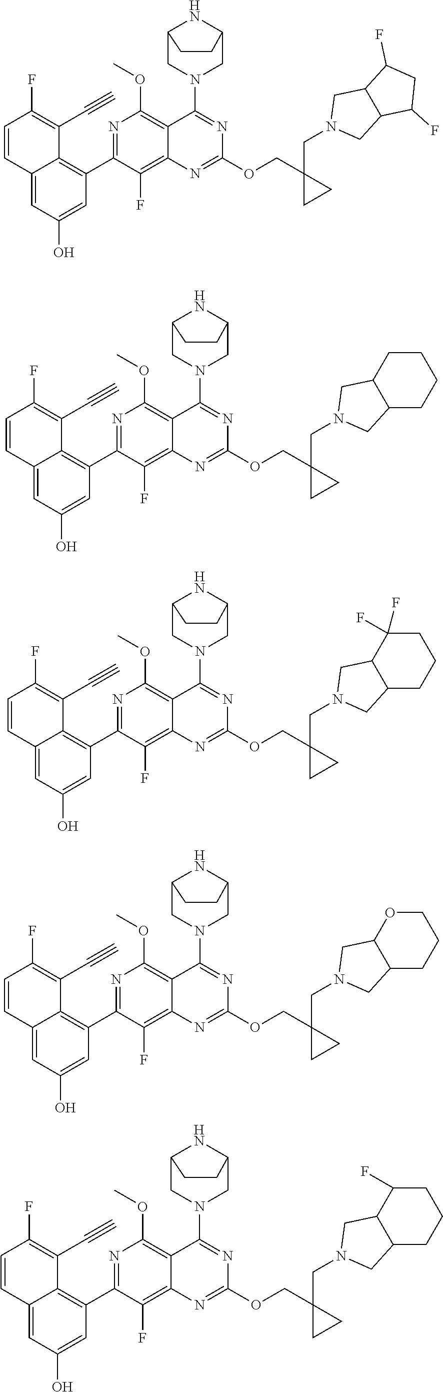 [SMILES: C#Cc1c(F)ccc2cc(O)cc(-c3nc(OC)c4c(N5CC6CCC(C5)N6)nc(OCC5(CN6CC7C(F)CC(F)C7C6)CC5)nc4c3F)c12 ; C#Cc1c(F)ccc2cc(O)cc(-c3nc(OC)c4c(N5CC6CCC(C5)N6)nc(OCC5(CN6CC7CCCCC7C6)CC5)nc4c3F)c12 ; C#Cc1c(F)ccc2cc(O)cc(-c3nc(OC)c4c(N5CC6CCC(C5)N6)nc(OCC5(CN6CC7CCCC(F)(F)C7C6)CC5)nc4c3F)c12 ; C#Cc1c(F)ccc2cc(O)cc(-c3nc(OC)c4c(N5CC6CCC(C5)N6)nc(OCC5(CN6CC7CCCOC7C6)CC5)nc4c3F)c12 ; C#Cc1c(F)ccc2cc(O)cc(-c3nc(OC)c4c(N5CC6CCC(C5)N6)nc(OCC5(CN6CC7CCCC(F)C7C6)CC5)nc4c3F)c12]

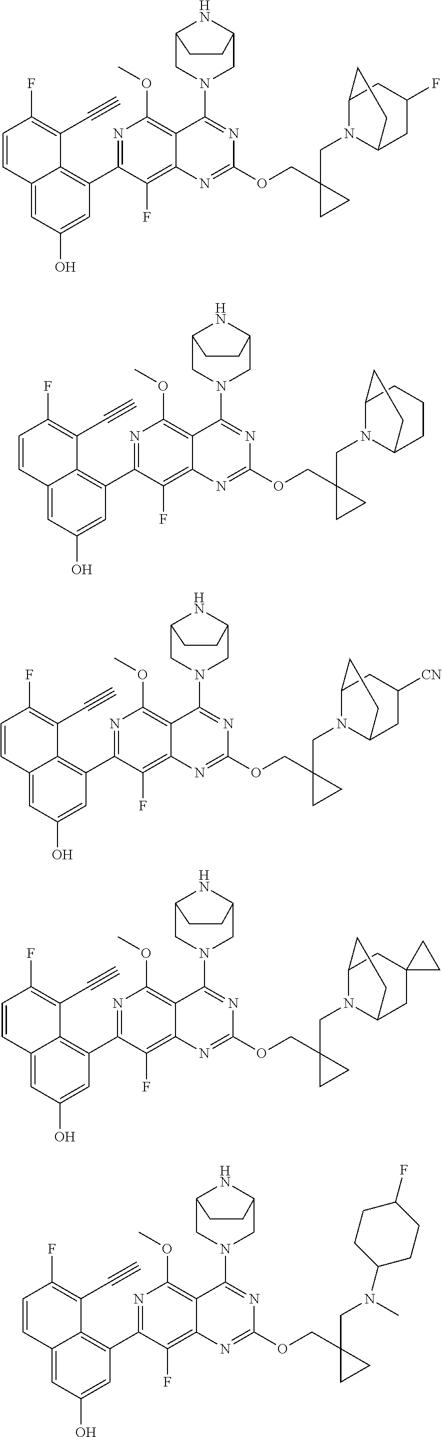 [SMILES: C#Cc1c(F)ccc2cc(O)cc(-c3nc(OC)c4c(N5CC6CCC(C5)N6)nc(OCC5(CN6C7CCC6CC(F)C7)CC5)nc4c3F)c12 ; C#Cc1c(F)ccc2cc(O)cc(-c3nc(OC)c4c(N5CC6CCC(C5)N6)nc(OCC5(CN6C7CCCC6CC7)CC5)nc4c3F)c12 ; C#Cc1c(F)ccc2cc(O)cc(-c3nc(OC)c4c(N5CC6CCC(C5)N6)nc(OCC5(CN6C7CCC6CC(C#N)C7)CC5)nc4c3F)c12 ; C#Cc1c(F)ccc2cc(O)cc(-c3nc(OC)c4c(N5CC6CCC(C5)N6)nc(OCC5(CN6C7CCC6CC6(CC6)C7)CC5)nc4c3F)c12 ; C#Cc1c(F)ccc2cc(O)cc(-c3nc(OC)c4c(N5CC6CCC(C5)N6)nc(OCC5(CN(C)C6CCC(F)CC6)CC5)nc4c3F)c12]

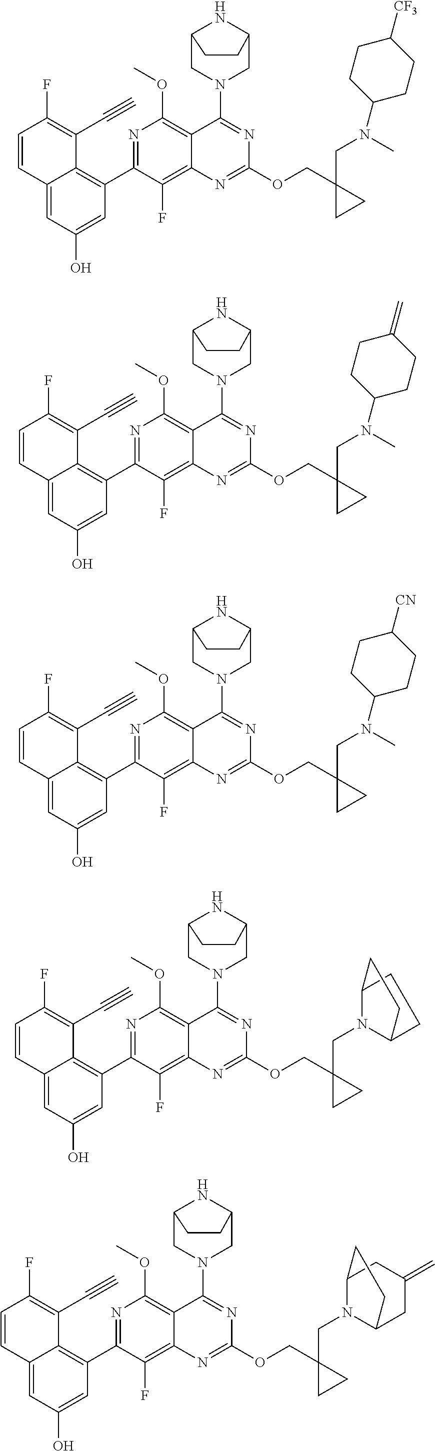 [SMILES: C#Cc1c(F)ccc2cc(O)cc(-c3nc(OC)c4c(N5CC6CCC(C5)N6)nc(OCC5(CN(C)C6CCC(C(F)(F)F)CC6)CC5)nc4c3F)c12 ; C#Cc1c(F)ccc2cc(O)cc(-c3nc(OC)c4c(N5CC6CCC(C5)N6)nc(OCC5(CN(C)C6CCC(=C)CC6)CC5)nc4c3F)c12 ; C#Cc1c(F)ccc2cc(O)cc(-c3nc(OC)c4c(N5CC6CCC(C5)N6)nc(OCC5(CN(C)C6CCC(C#N)CC6)CC5)nc4c3F)c12 ; C#Cc1c(F)ccc2cc(O)cc(-c3nc(OC)c4c(N5CC6CCC(C5)N6)nc(OCC5(CN6C7CCC6CC7)CC5)nc4c3F)c12 ; C#Cc1c(F)ccc2cc(O)cc(-c3nc(OC)c4c(N5CC6CCC(C5)N6)nc(OCC5(CN6C7CCC6CC(=C)C7)CC5)nc4c3F)c12]

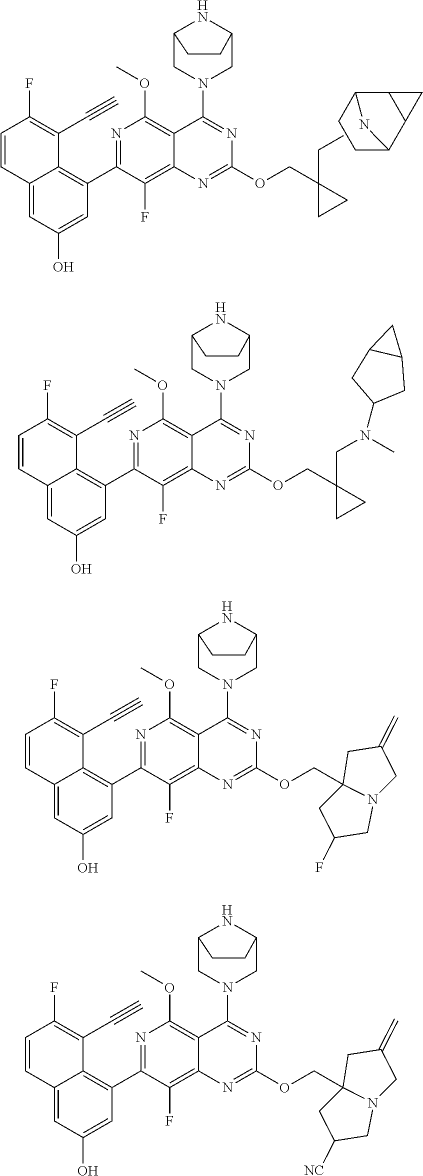

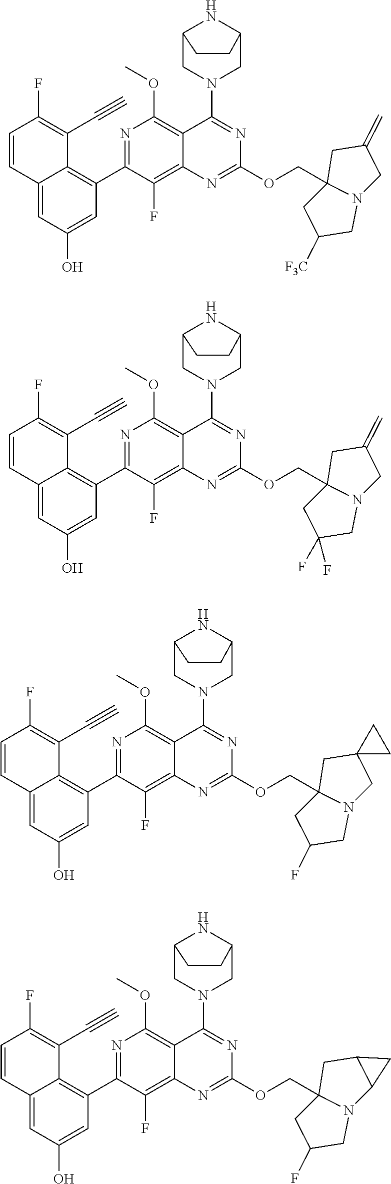 [SMILES: C#Cc1c(F)ccc2cc(O)cc(-c3nc(OC)c4c(N5CC6CCC(C5)N6)nc(OCC56CC(=C)CN5CC(C(F)(F)F)C6)nc4c3F)c12 ; C#Cc1c(F)ccc2cc(O)cc(-c3nc(OC)c4c(N5CC6CCC(C5)N6)nc(OCC56CC(=C)CN5CC(F)(F)C6)nc4c3F)c12 ; C#Cc1c(F)ccc2cc(O)cc(-c3nc(OC)c4c(N5CC6CCC(C5)N6)nc(OCC56CC(F)CN5CC5(CC5)C6)nc4c3F)c12 ; C#Cc1c(F)ccc2cc(O)cc(-c3nc(OC)c4c(N5CC6CCC(C5)N6)nc(OCC56CC(F)CN5C5CC5C6)nc4c3F)c12]

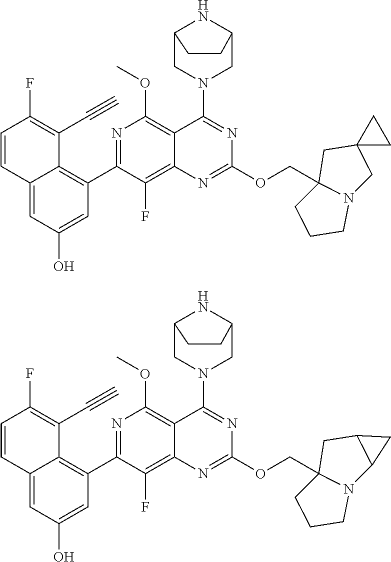 [SMILES: C#Cc1c(F)ccc2cc(O)cc(-c3nc(OC)c4c(N5CC6CCC(C5)N6)nc(OCC56CCCN5CC5(CC5)C6)nc4c3F)c12 ; C#Cc1c(F)ccc2cc(O)cc(-c3nc(OC)c4c(N5CC6CCC(C5)N6)nc(OCC56CCCN5C5CC5C6)nc4c3F)c12]

or a pharmaceutically acceptable salt of any one of the foregoing.

### Biological Assays/Testing

### Cell Lines

The following cancer cell lines were employed: AGS gastric carcinoma [heterozygous G12D] (ATCC, CRL-1739); A-427 lung carcinoma [heterozygous G12D] (ATCC, HTB-53); ASPC₁ pancreatic adenocarcinoma [homozygous G12D] (ATCC, CRL-1682) and SW1990 pancreatic adenocarcinoma [homozygous G12D] (ATCC CRL-2172). Cell lines were cultured essentially according to ATCC recommendations.

### KRAS(G12D)/SOS1 Homogeneous Time-Resolved Fluorescence (HTRF) Assay

Binding of test compounds to KRAS(G12D) target protein, which in turn blocks KRAS(G12D) interaction with the SOS1 protein, was measured in the absences of GTP by homogeneous time-resolved fluorescence using the KRAS-G12D/SOS1 Binding Assay Kit (Cisbio, 63ADK000CB17PEH), following the manufacturer's instructions, except as noted. 3-fold serial dilutions of each test compound were prepared ranging from 20 μM to 1.02 nM. The test compound was mixed and incubated with reaction components, incubated in a sealed plate at 4° C. for 3 hr and fluorescence was measured using a PerkinElmer Envision plate reader. The KRAS(G12D)-SOS1 IC₅₀ values (the concentration of 50% of the maximal inhibition) were calculated using GraphPad Prism 7 software. Results are listed in Cancer cell line proliferation (CellTiter-Glo® assays)

AGS, A-427, ASPC₁, SW 1990, and GP2D cells were plated in 96-well tissue culture plates at 4,000 cells/well and incubated at 37° C./5% CO2 for 72 hr in 100 μl of media. 3-fold serial dilutions of each test compound were prepared ranging from 20 μM to 1.02 nM. Each cell line was then treated with test compounds at various concentrations with a final concentration of 0.5% DMSO/well, and then incubated at 37° C./5% CO2 for 72 hr. 100 μl of CellTiter-Glo® Reagent (Promega Corporation, Madison, WI) was added to each well and processed according manufacturer's protocol. Results were analyzed and IC50 values were calculated in GraphPad 7 software. Results are listed in Table 2. KRAS(G12D)/SOS1 HTRF assay: A. IC_{50<100} nM; B. IC₅₀₌₁₀₀-1000 nM; C. IC_{50>1000} nM; AGS proliferation assay: A. EC_{50<100} nM; B. EC₅₀-100-1000 nM; C. EC_{50>1000} nM; A427 proliferation assay: A. EC_{50<100} nM; B. EC₅₀-100-1000 nM; C. EC_{50>1000} nM; ASPC proliferation assay: A. EC_{50<100} nM; B. EC₅₀₌₁₀₀-1000 nM; C. EC_{50>1000} nM; SW1990 proliferation assay: A. EC_{50<100} nM; B. EC₅₀₌₁₀₀-1000 nM; C. EC_{50>1000} nM.

TABLE 2
Biochemical and Cell-based Assays of Compounds

| | HTRF of | | | | |
|---|---|---|---|---|---|
| | KRAS^{G12D}- | CTG(IC50); ABS-72 H(nM) | | | |
| Ex | SOS1 | AGS | A427 | ASPC-1 | SW1990 |
| Ref. 1 | A | A | A | B | A |
| Ref. 2 | A | A | — | B | B |
| Ref. 3 | | A | | | |
| Ref. 4 | | A | | | |
| 1 | A | A | A | A | A |
| 2 | A | A | A | A | A |
| 3 | A | A | A | A | A |
| 4 | A | A | A | A | A |
| 5 | A | A | A | A | — |
| 6 | A | A | A | B | — |
| 7 | A | A | A | A | A |
| 8 | A | A | A | A | A |
| 9 | A | A | B | B | — |
| 10 | A | A | A | A | — |
| 11 | A | A | A | A | — |
| 12 | A | A | B | B | — |
| 13 | A | A | A | A | A |
| 14 | A | A | A | A | A |
| 15 | A | A | A | A | — |
| 16 | A | A | A | A | — |
| 17 | A | A | B | — | — |
| 18 | A | A | B | — | — |
| 19 | A | A | A | A | A |
| 20 | A | A | B | A | A |
| 21 | A | A | B | B | A |
| 22 | B | C | C | C | — |
| 23 | B | C | C | C | C |
| 24 | A | A | B | C | A |
| 25 | A | A | C | B | B |
| 26 | A | A | B | A | A |
| 27 | A | A | B | B | A |
| 28 | A | A | B | A | A |
| 29 | A | A | B | A | A |
| 30 | A | A | A | A | A |
| 31 | A | A | B | A | A |
| 32 | A | A | B | B | A |
| 33 | A | A | B | A | A |
| 34 | A | A | B | A | A |
| 35 | A | A | | A | A |
| 36 | A | A | B | A | A |
| 37 | A | A | B | A | A |
| 38 | A | A | — | A | A |
| 39 | A | A | B | A | A |
| 40 | A | A | B | A | A |
| 41 | A | A | B | A | A |
| 42 | A | A | B | A | A |
| 43 | A | A | B | A | A |
| 44 | A | A | B | A | A |
| 45 | A | B | B | B | B |
| 46 | A | A | — | B | A |
| 47 | A | A | — | A | A |
| 48 | A | A | — | A | A |
| 49 | A | A | — | A | A |
| 50 | A | A | — | A | A |
| 51 | A | A | — | A | A |
| 52 | A | A | B | A | A |

### Oral Bioavailability

CD-1 mice were randomly assigned to 6 groups with 3 male mice in each compound group. Control groups included reference compound 1 (50 mg/kg), reference compound 2 (25 mg/kg) and example 1 (25 mg/kg). Inventive compound groups included prodrug compound of Example 52 (50 mg/kg), compound of example 2 (50 mg/kg), compound of example 3 (50 mg/kg) and compound of example 4 (25 mg/kg). Compounds were orally administered (PO) in a single dose to each mouse in its group. Blood samples were taken within 72 hours. Bioavailability (F %) was determined and by liquid chromatography-mass spectrometry (LC-MS/MS). The mean oral % F is provided in Table 3.

TABLE 3

| Ex | Structure | % F |
|---|---|---|
| Ref 1 | 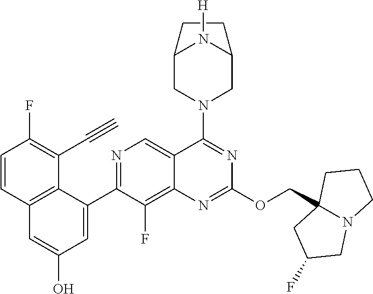 | 0.45 |
| Ref 2 | 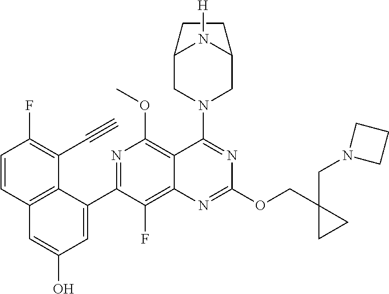 [SMILES: C#Cc1c(F)ccc2cc(O)cc(-c3nc(OC)c4c(N5CC6CCC(C5)N6)nc(OCC5(CN6CCC6)CC5)nc4c3F)c12] | 0.15 |
| Ref. 3 | 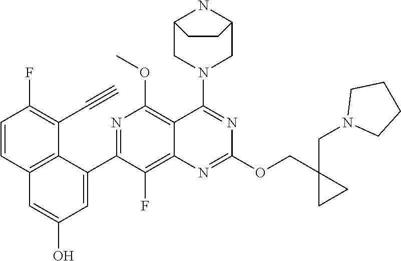 [SMILES: C#Cc1c(F)ccc2cc(O)cc(-c3nc(OC)c4c(N5CC6CCC(C5)N6)nc(OCC5(CN6CCCC6)CC5)nc4c3F)c12] | 0.23 |
| Ref. 4 | 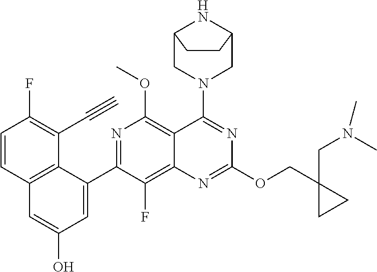 [SMILES: C#Cc1c(F)ccc2cc(O)cc(-c3nc(OC)c4c(N5CC6CCC(C5)N6)nc(OCC5(CN(C)C)CC5)nc4c3F)c12] | 0.23 |
| 1 | 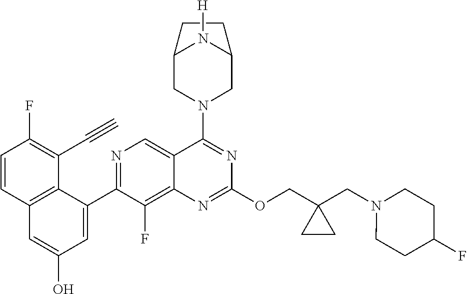 [SMILES: C#Cc1c(F)ccc2cc(O)cc(-c3ncc4c(N5CC6CCC(C5)N6)nc(OCC5(CN6CCC(F)CC6)CC5)nc4c3F)c12] | 0.8 |
| 2 | 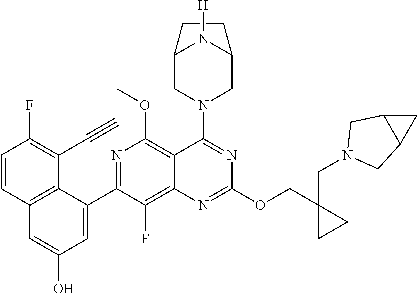 [SMILES: C#Cc1c(F)ccc2cc(O)cc(-c3nc(OC)c4c(N5CC6CCC(C5)N6)nc(OCC5(CN6CC7CC7C6)CC5)nc4c3F)c12] | 10.6 |
| 3 | 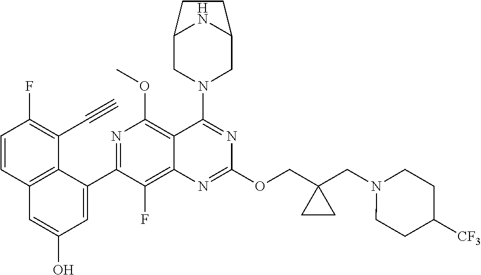 [SMILES: C#Cc1c(F)ccc2cc(O)cc(-c3nc(OC)c4c(N5CC6CCC(C5)N6)nc(OCC5(CN6CCC(C(F)(F)F)CC6)CC5)nc4c3F)c12] | 13.1 |
| 4 | 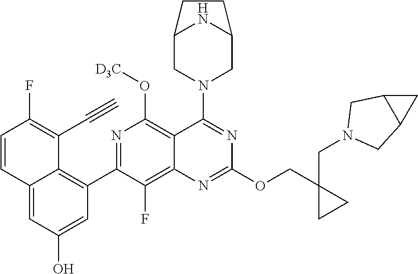 [SMILES: [2H]C([2H])([2H])Oc1nc(-c2cc(O)cc3ccc(F)c(C#C)c23)c(F)c2nc(OCC3(CN4CC5CC5C4)CC3)nc(N3CC4CCC(C3)N4)c12] | 5.4 |
| 52 | 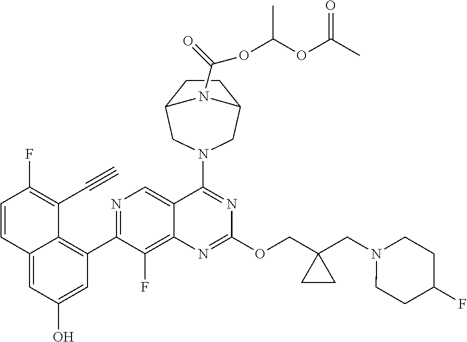 [SMILES: C#Cc1c(F)ccc2cc(O)cc(-c3ncc4c(N5CC6CCC(C5)N6C(=O)OC(C)OC(C)=O)nc(OCC5(CN6CCC(F)CC6)CC5)nc4c3F)c12] | 10.1 |
| 13 | 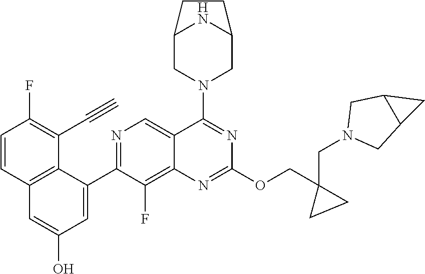 [SMILES: C#Cc1c(F)ccc2cc(O)cc(-c3ncc4c(N5CC6CCC(C5)N6)nc(OCC5(CN6CC7CC7C6)CC5)nc4c3F)c12] | 1.98 |
| 14 | 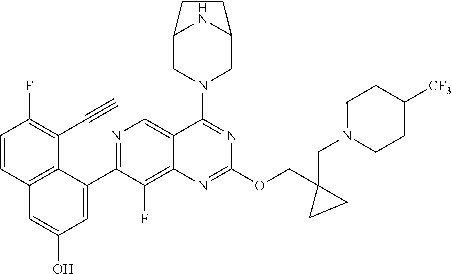 [SMILES: C#Cc1c(F)ccc2cc(O)cc(-c3ncc4c(N5CC6CCC(C5)N6)nc(OCC5(CN6CCC(C(F)(F)F)CC6)CC5)nc4c3F)c12] | 5.81 |
| 19 | 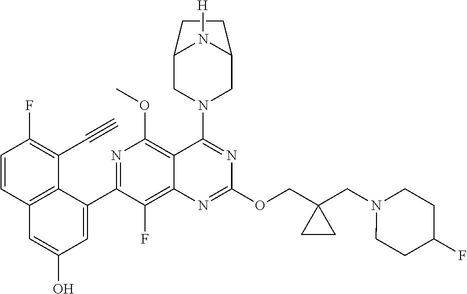 [SMILES: C#Cc1c(F)ccc2cc(O)cc(-c3nc(OC)c4c(N5CC6CCC(C5)N6)nc(OCC5(CN6CCC(F)CC6)CC5)nc4c3F)c12] | 9.39 |
| 34 | 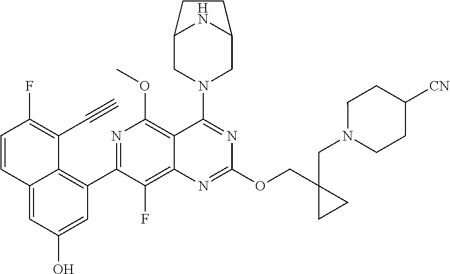 [SMILES: C#Cc1c(F)ccc2cc(O)cc(-c3nc(OC)c4c(N5CC6CCC(C5)N6)nc(OCC5(CN6CCC(C#N)CC6)CC5)nc4c3F)c12] | 1.03 |
| 37 | 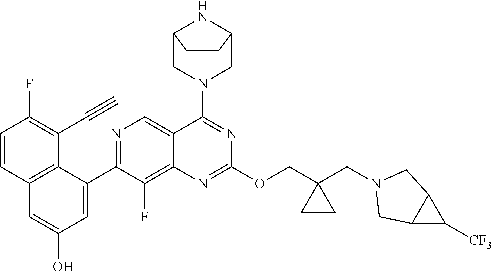 [SMILES: C#Cc1c(F)ccc2cc(O)cc(-c3ncc4c(N5CC6CCC(C5)N6)nc(OCC5(CN6CC7C(C6)C7C(F)(F)F)CC5)nc4c3F)c12] | 10.6 |
| 38 | 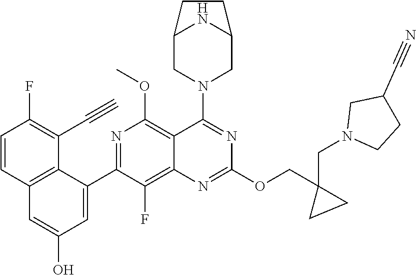 [SMILES: C#Cc1c(F)ccc2cc(O)cc(-c3nc(OC)c4c(N5CC6CCC(C5)N6)nc(OCC5(CN6CCC(C#N)C6)CC5)nc4c3F)c12] | 1.8 |
| 43 | 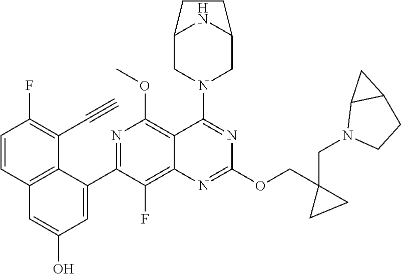 [SMILES: C#Cc1c(F)ccc2cc(O)cc(-c3nc(OC)c4c(N5CC6CCC(C5)N6)nc(OCC5(CN6CCC7CC76)CC5)nc4c3F)c12] | 2.37 |
| 44 | 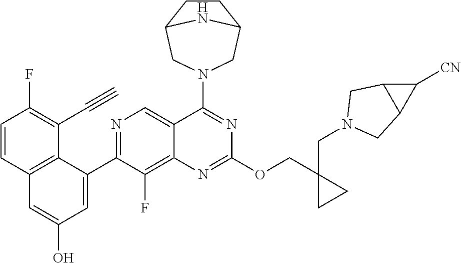 [SMILES: C#Cc1c(F)ccc2cc(O)cc(-c3ncc4c(N5CC6CCC(C5)N6)nc(OCC5(CN6CC7C(C#N)C7C6)CC5)nc4c3F)c12] | 2.38 |
| 49 | 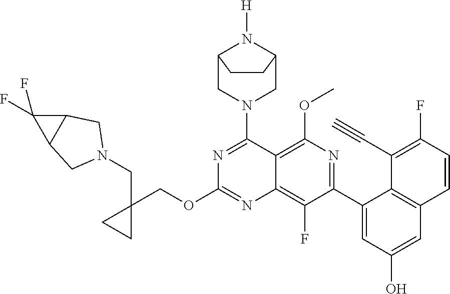 [SMILES: C#Cc1c(F)ccc2cc(O)cc(-c3nc(OC)c4c(N5CC6CCC(C5)N6)nc(OCC5(CN6CC7C(C6)C7(F)F)CC5)nc4c3F)c12] | 3.82 |
| 51 | 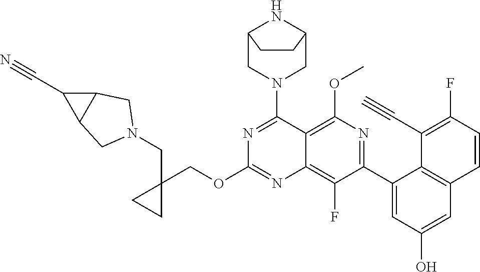 [SMILES: C#Cc1c(F)ccc2cc(O)cc(-c3nc(OC)c4c(N5CC6CCC(C5)N6)nc(OCC5(CN6CC7C(C#N)C7C6)CC5)nc4c3F)c12] | 2.3 |
| 100 | 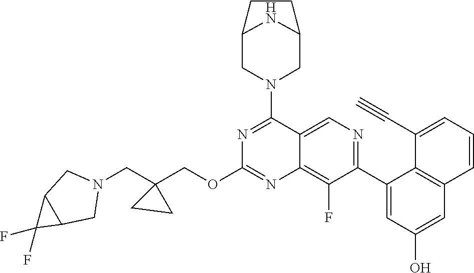 [SMILES: C#Cc1cccc2cc(O)cc(-c3ncc4c(N5CC6CCC(C5)N6)nc(OCC5(CN6CC7C(C6)C7(F)F)CC5)nc4c3F)c12] | 1.70 |
| 146 | 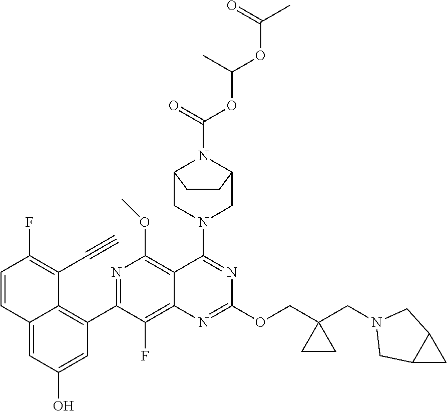 [SMILES: C#Cc1c(F)ccc2cc(O)cc(-c3nc(OC)c4c(N5CC6CCC(C5)N6C(=O)OC(C)OC(C)=O)nc(OCC5(CN6CC7CC7C6)CC5)nc4c3F)c12] | 8.3 |
| 148 | 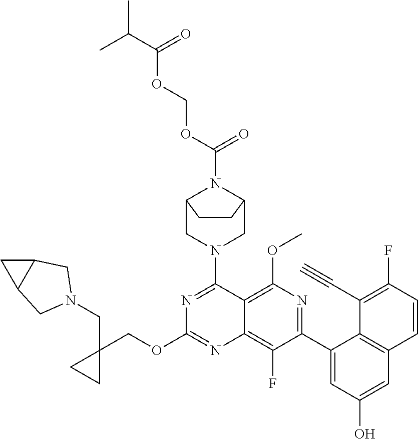 [SMILES: C#Cc1c(F)ccc2cc(O)cc(-c3nc(OC)c4c(N5CC6CCC(C5)N6C(=O)OCOC(=O)C(C)C)nc(OCC5(CN6CC7CC7C6)CC5)nc4c3F)c12] | 6.8 |
| 149 | 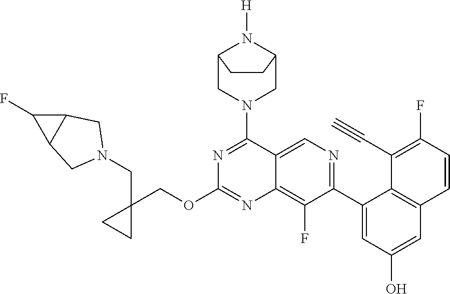 [SMILES: C#Cc1c(F)ccc2cc(O)cc(-c3ncc4c(N5CC6CCC(C5)N6)nc(OCC5(CN6CC7C(F)C7C6)CC5)nc4c3F)c12] | 12.9 |
| 155 | 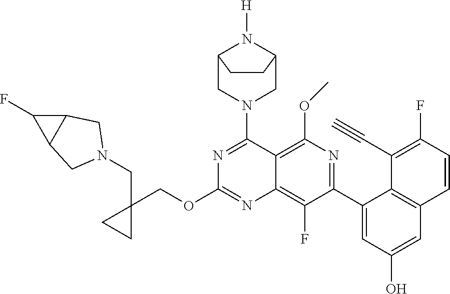 [SMILES: C#Cc1c(F)ccc2cc(O)cc(-c3nc(OC)c4c(N5CC6CCC(C5)N6)nc(OCC5(CN6CC7C(F)C7C6)CC5)nc4c3F)c12] | 8.03 |
| 165 | 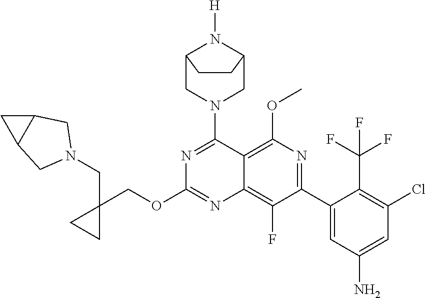 [SMILES: COc1nc(-c2cc(N)cc(Cl)c2C(F)(F)F)c(F)c2nc(OCC3(CN4CC5CC5C4)CC3)nc(N3CC4CCC(C3)N4)c12] | 13.8 |
| 167 | 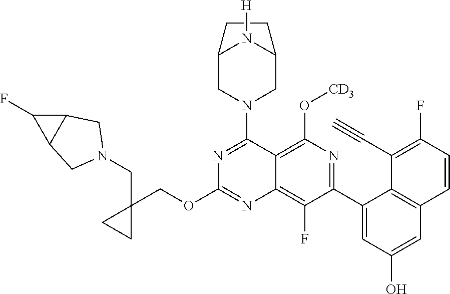 [SMILES: [2H]C([2H])([2H])Oc1nc(-c2cc(O)cc3ccc(F)c(C#C)c23)c(F)c2nc(OCC3(CN4CC5C(F)C5C4)CC3)nc(N3CC4CCC(C3)N4)c12] | 7.14 |
| 168 | 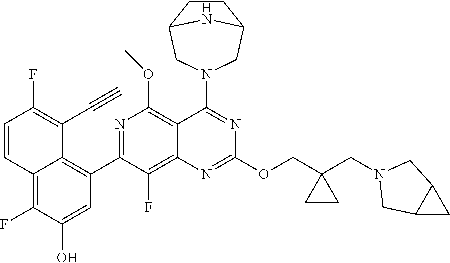 [SMILES: C#Cc1c(F)ccc2c(F)c(O)cc(-c3nc(OC)c4c(N5CC6CCC(C5)N6)nc(OCC5(CN6CC7CC7C6)CC5)nc4c3F)c12] | 13.6 |
| 184 | 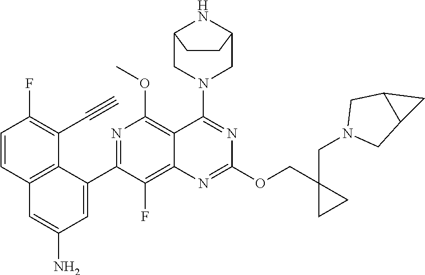 [SMILES: C#Cc1c(F)ccc2cc(N)cc(-c3nc(OC)c4c(N5CC6CCC(C5)N6)nc(OCC5(CN6CC7CC7C6)CC5)nc4c3F)c12] | 5.75 |

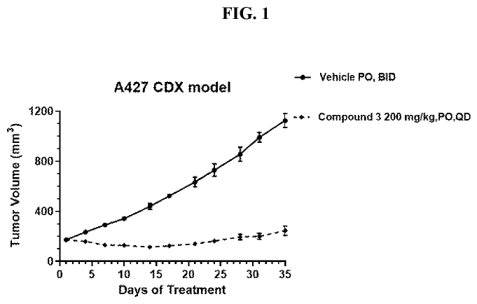

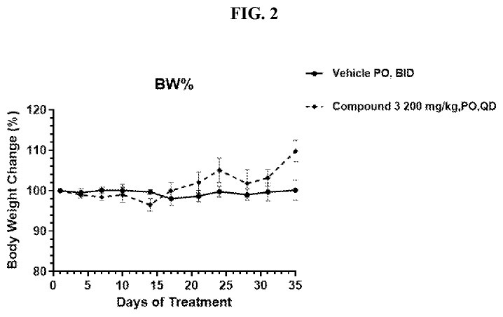

Compound 3 was evaluated in the human lung carcinoma A427 xenograft model using female NOD SCID mice (6-8 weeks old). Each mouse was inoculated subcutaneously in the right flank with A427 tumor cells (1×10⁷) in 0.1 ml of Medium and Matrigel mixture (1:1 ratio) to initiate tumor development. Once tumors reached an average size of ˜170 mm³, mice were randomized among treatment groups followed by administration of test articles or vehicle. Compound 3 was administrated by oral gavage (PO) once daily at 200 mg/kg for 5 weeks. And vehicle was administrated by oral gavage twice daily. Body weight and tumor volume was measured twice weekly until the study finished. The results are illustrated in FIG. 1 and FIG. 2.

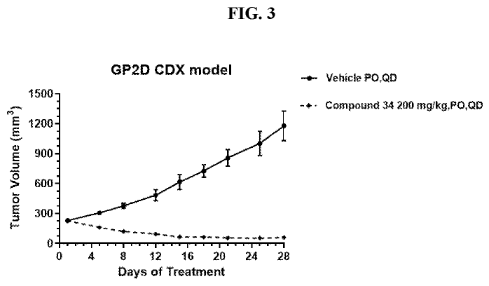

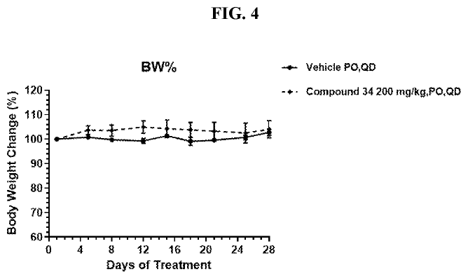

Compound 34 was evaluated in the human colon adenocarcinoma GP2D xenograft model using female BALB/c nude mice (6-8 weeks old). Each mouse was inoculated subcutaneously on the right flank with GP2D tumor cells (1×10⁷) in 0.1 ml of Medium and Matrigel mixture (1:1 ratio) to initiate tumor development. Once tumors reached an average size of ˜230 mm³, mice were randomized among treatment groups followed by administration of test articles or vehicle. Compound 34 was administrated by oral gavage (PO) once daily at 200 mg/kg for 4 weeks and vehicle was administrated by oral gavage once daily for 4 weeks. Body weight and tumor volume was measured twice weekly until the study finished. The results are illustrated in FIG. 3 and FIG. 4.

Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

All patents and publications mentioned in this specification are herein incorporated by reference to the same extent as if each independent patent and publication was specifically and individually indicated to be incorporated by reference.

1. A compound of the Formula I: 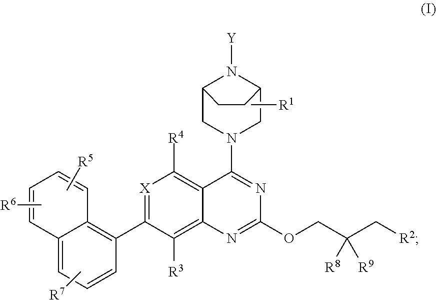
or a pharmaceutically acceptable salt thereof, wherein
Y is hydrogen or —C(O)OCHR^{a}OC(O)R^{b};
X is CH or N;
R¹ is hydrogen, halo, OH, (C₁-C₄)alkyl, (C₁-C₄)haloalkyl, (C₁-C₄)hydroxyalkyl, —CHO, —C(O)OR^{b}, —C(O)ONR^{a}R^{b} or a 5- to 6-membered heteroaryl optionally substituted with 1 to 3 groups selected from halo, (C₁-C₄)alkyl, (C₁-C₄)alkoxy, halo(C₁-C₄)alkoxy, and cyano;
R² is a 4- to 6-membered monocyclic heterocyclyl substituted with 1 to 3 groups selected from R^{c} or a 6- to 10-membered bicyclic heterocyclyl optionally substituted with 1 to 3 groups selected from R^{d},
R³ is selected from hydrogen, halo, (C₁-C₄)alkyl, cyano, and (C₃-C₆)cycloalkyl optionally substituted with 1 to 3 groups selected from halo, (C₁-C₄)alkyl, (C₁-C₄)alkoxy, halo(C₁-C₄)alkoxy, and cyano;
R⁴ is selected from hydrogen, (C₁-C₄)alkyl, (C₁-C₄)haloalkyl, (C₁-C₄)alkoxy, deuterated(C₁-C₄)alkoxy, (C₁-C₄)haloalkoxy, (C₁-C₄)alkynyl, (C₁-C₄)alkenyl, halo, (C₃-C₆)cycloalkyl, —O(C₃-C₆)cycloalkyl, cyano, NH₂, —NH(C₁-C₄)alkyl, —N[(C₁-C₄)alkyl]₂, —P(O)[(C₁-C₄)alkyl]₂, and —S(C₁-C₄)alkyl, wherein said (C₃-C₆)cycloalkyl and said (C₃-C₆)cycloalkyl of —O(C₃-C₆)cycloalkyl are optionally substituted with 1 to 3 groups selected from halo, (C₁-C₄)alkyl, (C₁-C₄)alkoxy, halo(C₁-C₄)alkoxy, and cyano;
R⁵ is (C₂-C₄)alkynyl;
R⁶ is hydrogen or halo;
R⁷ is hydrogen or OH;
R⁸ and R⁹ are taken together to form ═CH or cyclopropyl;
R^{a} and R^{b} are each independently selected from hydrogen and (C₁-C₄)alkyl; and
R^{c} and R^{d} are each independently selected from halo, (C₁-C₄)alkyl, (C₁-C₄)haloalkyl, (C₁-C₄)alkoxy, (C₁-C₄)haloalkoxy, cyano, OH, oxo, —C(O)OR^{a}, —C(O)R^{a}, —SO₂R^{a}, —S(O)R^{a}, —SO₂NR^{a}R^{b}, —NR^{a}C(O)R^{b}, —NR^{a}SO₂R^{b}, —NR^{a}R^{b}, and NO₂;
provided that R² is not piperazinyl substituted with (C₁-C₄)alkyl, —C(O)OR^{a}, or —C(O)R^{a} when R⁴ is (C₃)alkyl.

2. The compound of claim 1, wherein the compound is of the Formula II: 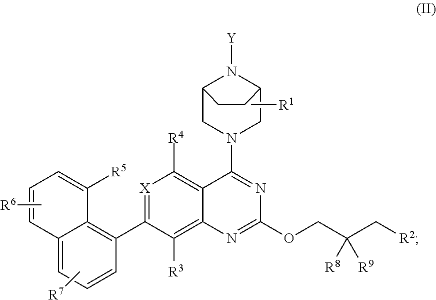
or a pharmaceutically acceptable salt thereof.

3. The compound of claim 1, wherein the compound is of the Formula III: 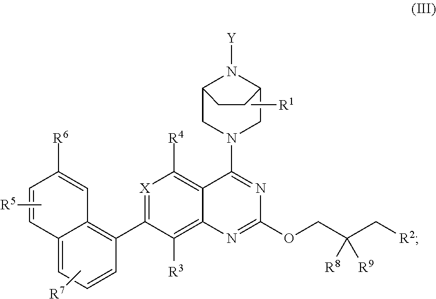
or a pharmaceutically acceptable salt thereof.

4. The compound of claim 1, wherein the compound is of the Formula IV: 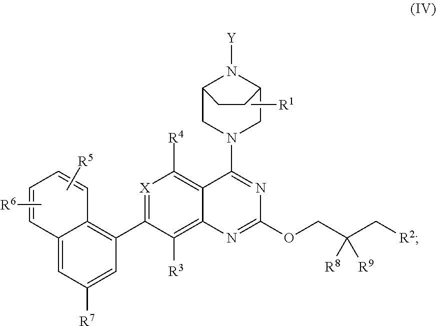
or a pharmaceutically acceptable salt thereof.

5. The compound of any one of claims 1 to 4, wherein the compound is of the Formula V: 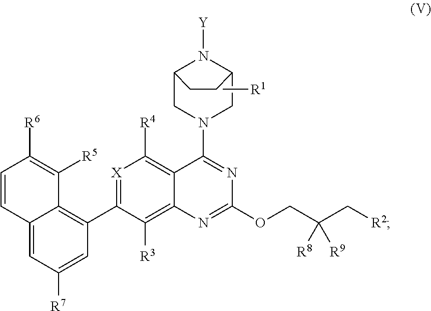
or a pharmaceutically acceptable salt thereof.

6. The compound of any one of claims 1 to 5, or a pharmaceutically acceptable salt thereof, wherein R¹ is hydrogen.

7. The compound of any one of claims 1 to 6, or a pharmaceutically acceptable salt thereof, wherein X is N.

8. The compound of any one of claims 1 to 7, or a pharmaceutically acceptable salt thereof, wherein R³ is halo.

9. The compound of any one of claims 1 to 8, or a pharmaceutically acceptable salt thereof, wherein R³ is fluoro.

10. The compound of any one of claims 1 to 9, or a pharmaceutically acceptable salt thereof, wherein R⁵ is (C₂)alkynyl.

11. The compound of any one of claims 1 to 10, or a pharmaceutically acceptable salt thereof, wherein R⁶ is halo.

12. The compound of any one of claims 1 to 11, or a pharmaceutically acceptable salt thereof, wherein R⁶ is fluoro.

13. The compound of any one of claims 1 to 12, or a pharmaceutically acceptable salt thereof, wherein R⁷ is OH.

14. The compound of any one of claims 1 to 13, or a pharmaceutically acceptable salt thereof, wherein R⁴ is selected from hydrogen, (C₁-C₄)alkoxy, deuterated (C₁-C₄)alkoxy, —N[(C₁-C₄)alkyl]₂, halo, (C₃-C₆)cycloalkyl, (C₁-C₄)haloalkoxy, (C₁-C₄)alkyl, and NH₂.

15. The compound of any one of claims 1 to 14, or a pharmaceutically acceptable salt thereof, wherein R⁴ is selected from hydrogen, methyl, methoxy, isopropoxy, OCDF₂, OCHF₂, —N(CH₃)₂, NH₂, chloro, and cyclopropyl.

16. The compound of any one of claims 1 to 15, or a pharmaceutically acceptable salt thereof, wherein R² is a 4- to 6-membered nitrogen containing monocyclic heterocyclyl substituted with 1 to 3 groups selected from R^{c} or a 7- to 10-membered nitrogen containing fused or spiro bicyclic heterocyclyl optionally substituted with 1 to 3 groups selected from R^{d}.

17. The compound of any one of claims 1 to 16, or a pharmaceutically acceptable salt thereof, wherein R² is azetidinyl, piperidinyl, morpholinyl, or pyrrolidinyl, each of which being substituted with 1 to 3 groups selected from R^{c} or R² is 3-azabicyclo[3.1.0]hexanyl, 2-azabicyclo[3.1.0]hexanyl, 1,4-dioxa-8-azaspiro[4.5]decanyl, or 1,2,3,6-tetrahydropyridinyl, each of which being optionally substituted with 1 to 3 groups selected from R^{d}.

18. The compound of any one of claims 1 to 17, or a pharmaceutically acceptable salt thereof, wherein R^{c} and R^{d} are each independently selected from halo, cyano, (C₁-C₄)haloalkyl, (C₁-C₄)alkoxy, (C₁-C₄)alkyl, (C₁-C₄)haloalkoxy, —S(O)R^{a}, and —SO₂NR^{a}R^{b}.

19. The compound of any one of claims 1 to 18, or a pharmaceutically acceptable salt thereof, wherein R^{c} and R^{d} are each independently selected from fluoro, cyano, CF₃, methoxy, isopropyl, OCF₃, —S(O)CH₃, and —SO₂N(CH₃)₂.

20. The compound of any one of claims 1 to 19, or a pharmaceutically acceptable salt thereof, wherein R⁸ and R⁹ are taken together to form cyclopropyl.

21. The compound of any one of claims 1 to 20, or a pharmaceutically acceptable salt thereof, wherein Y is hydrogen or —C(O)OCH(CH₃)OC(O)CH₃.

22. The compound of any one of claims 1 to 21, or a pharmaceutically acceptable salt thereof, wherein Y is hydrogen.

23. The compound of claim 1, wherein the compound is selected from 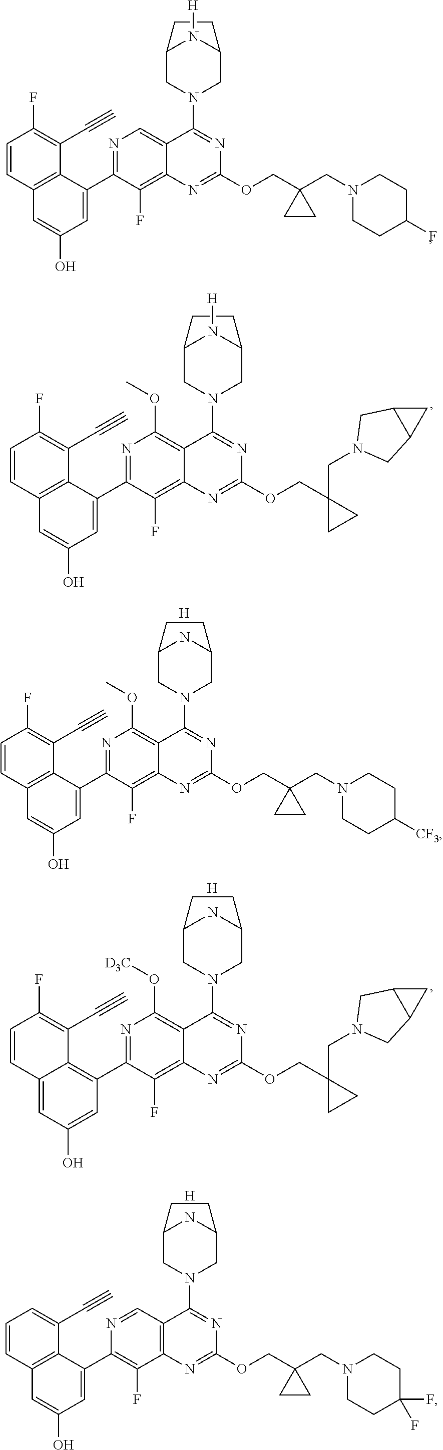 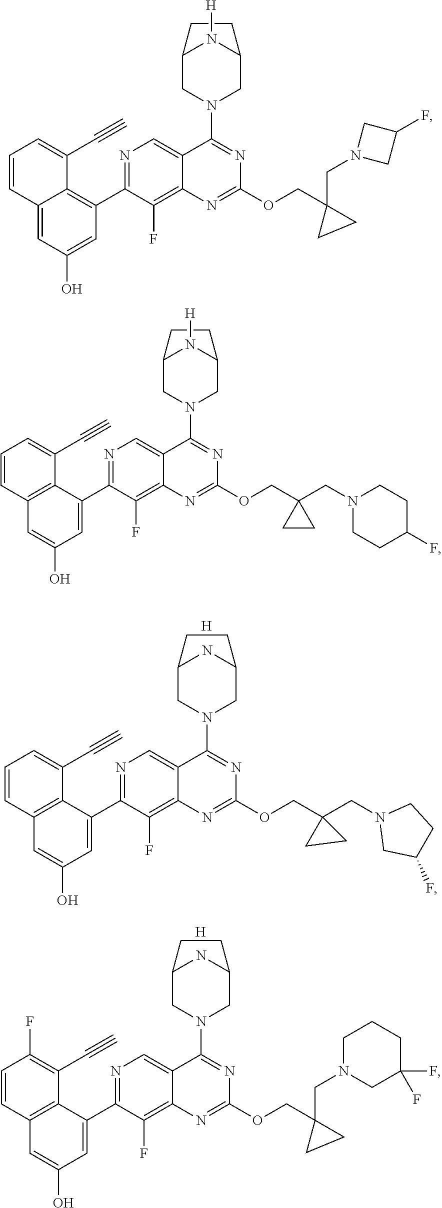 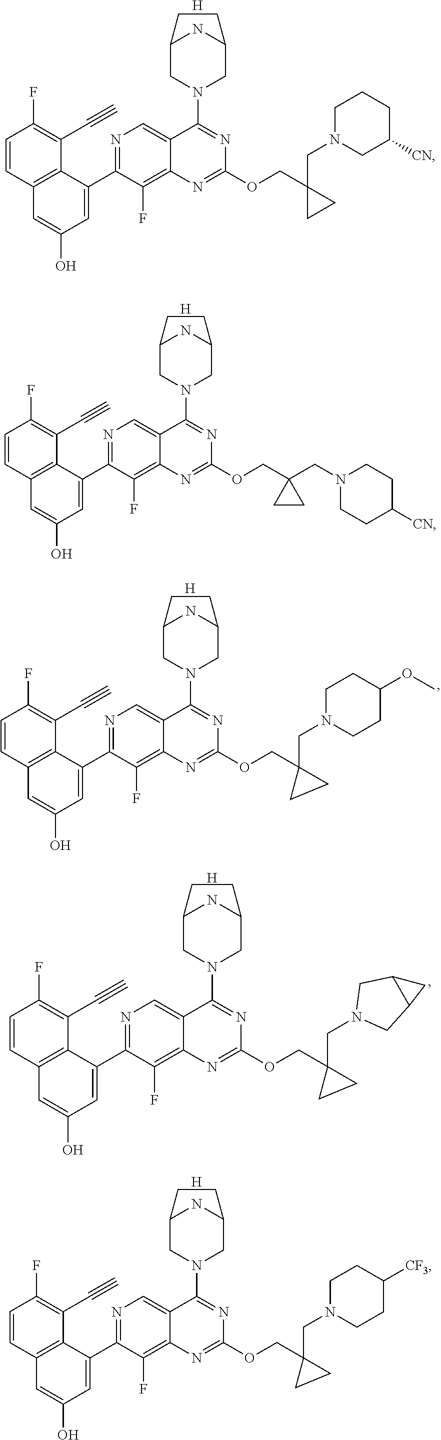 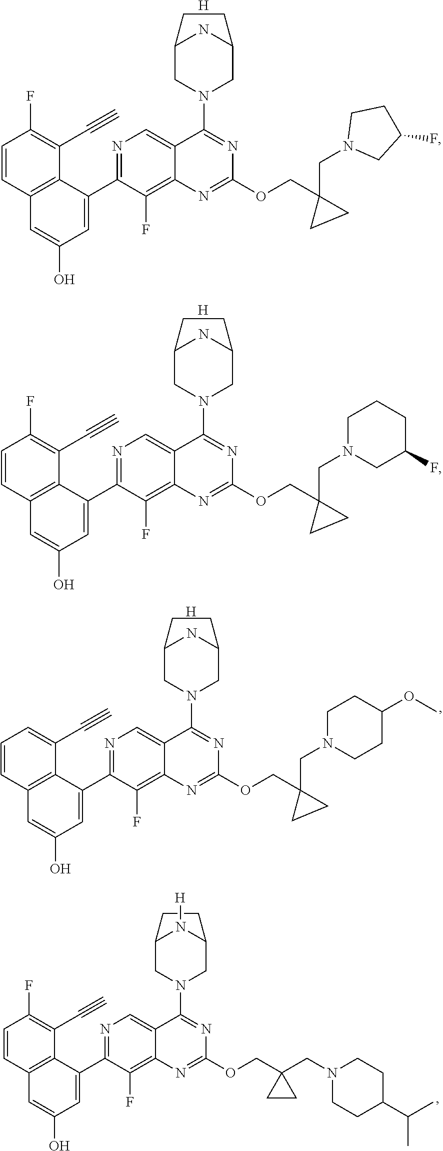 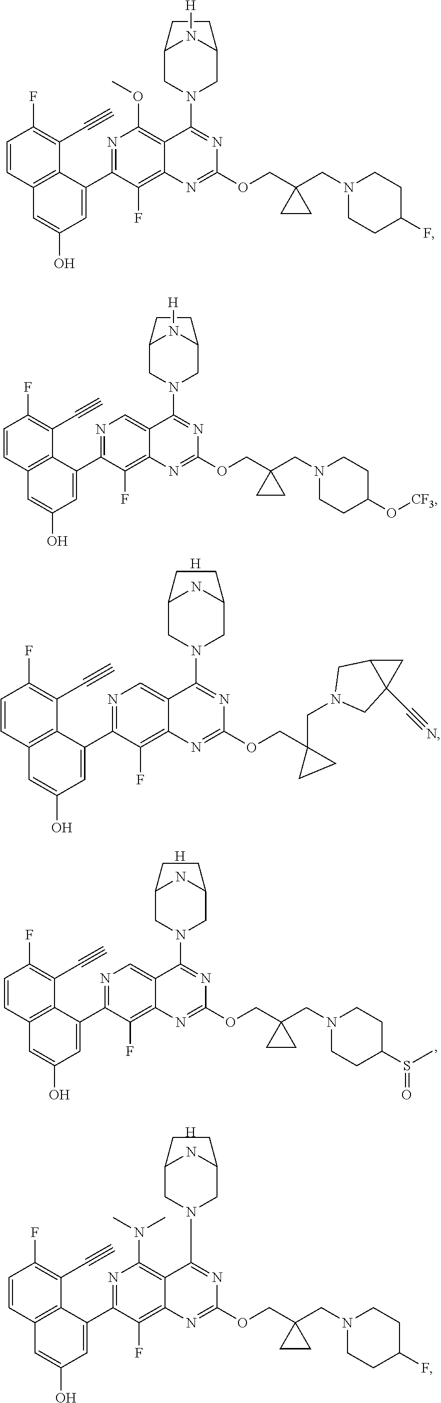 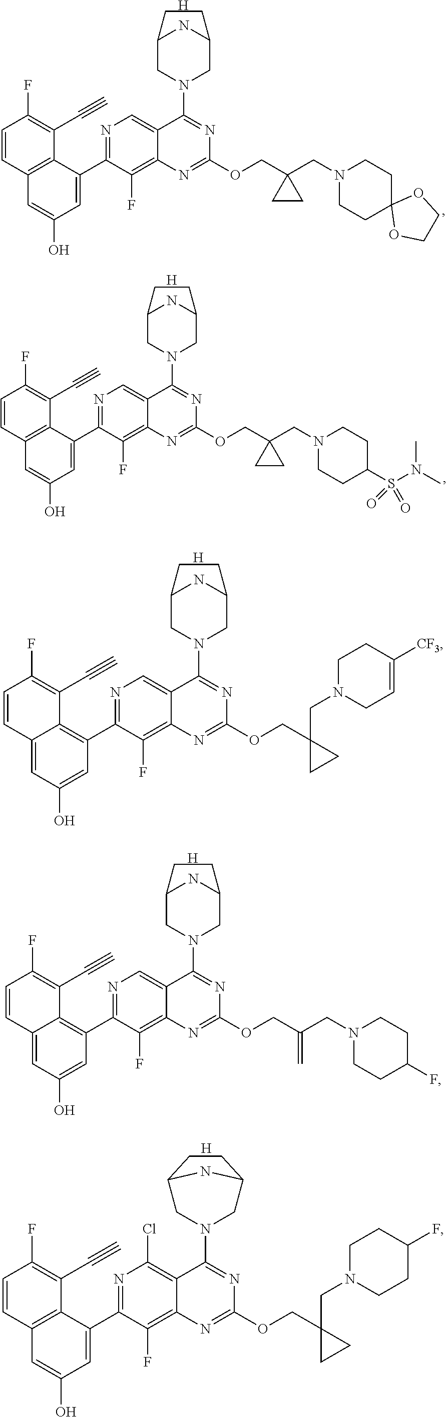 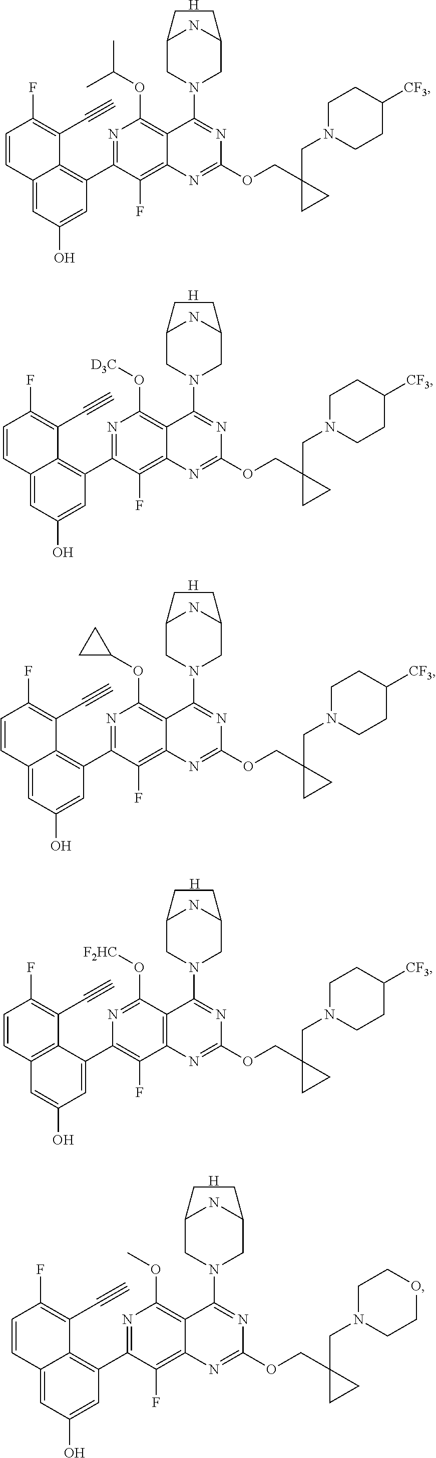 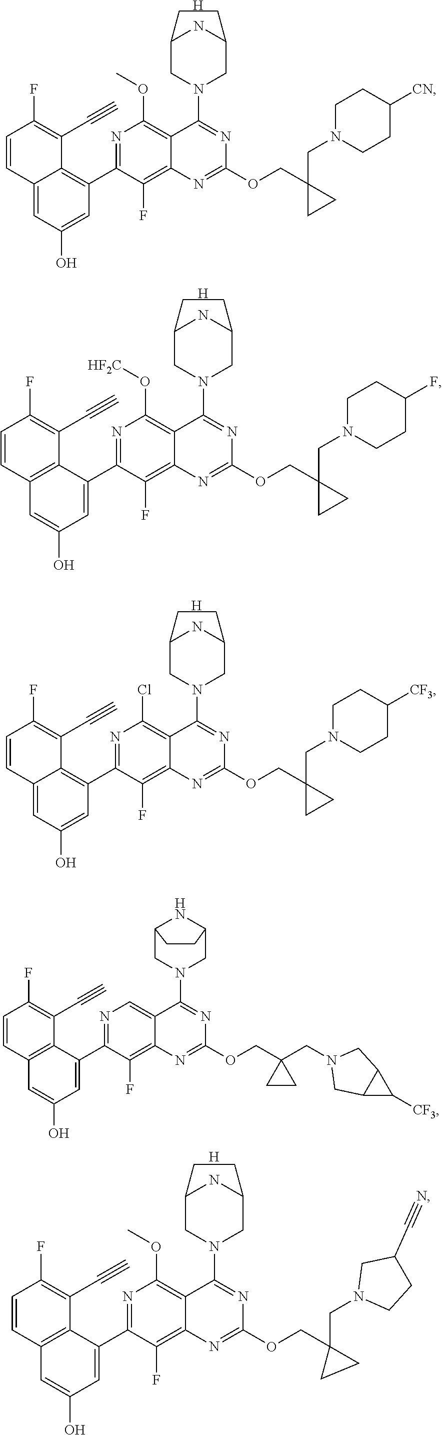 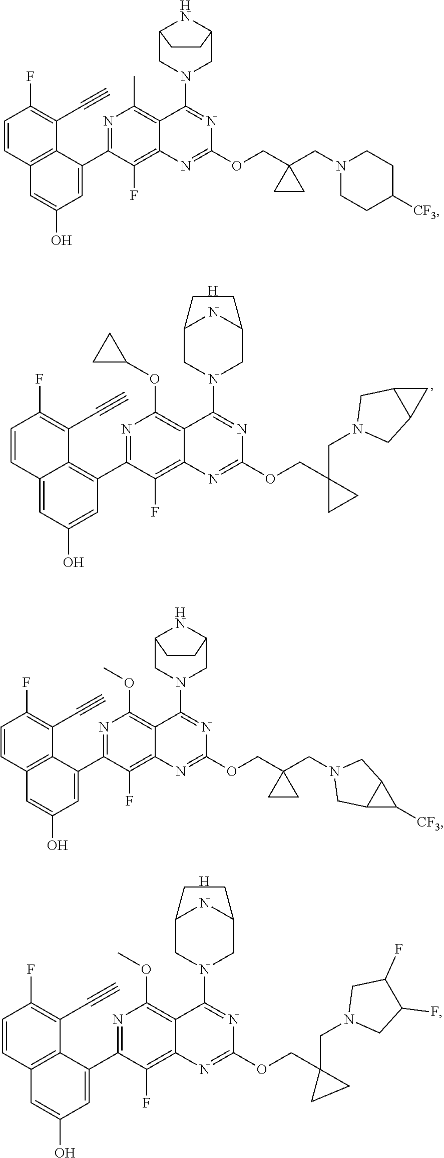 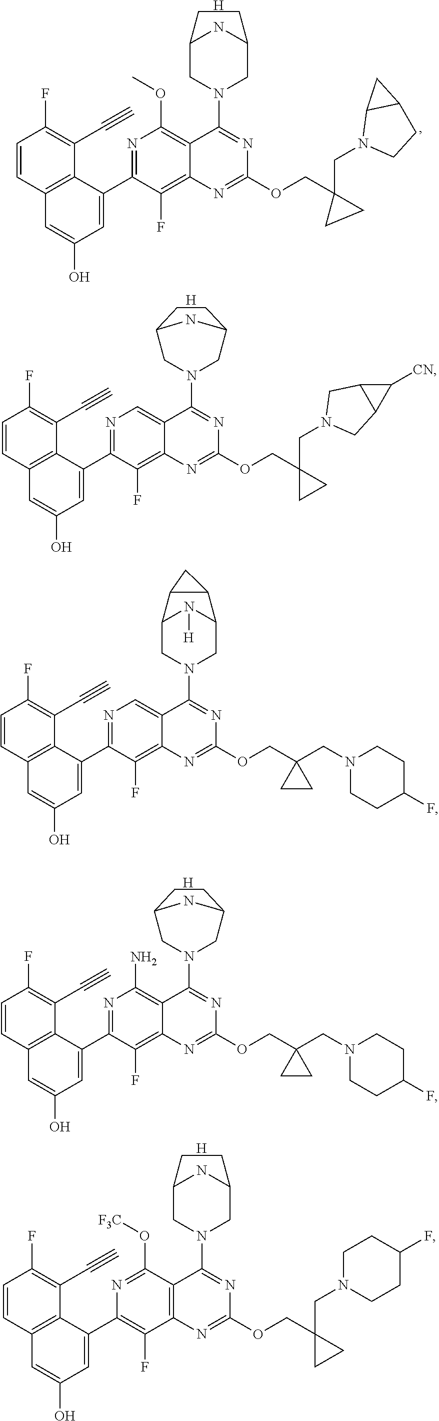 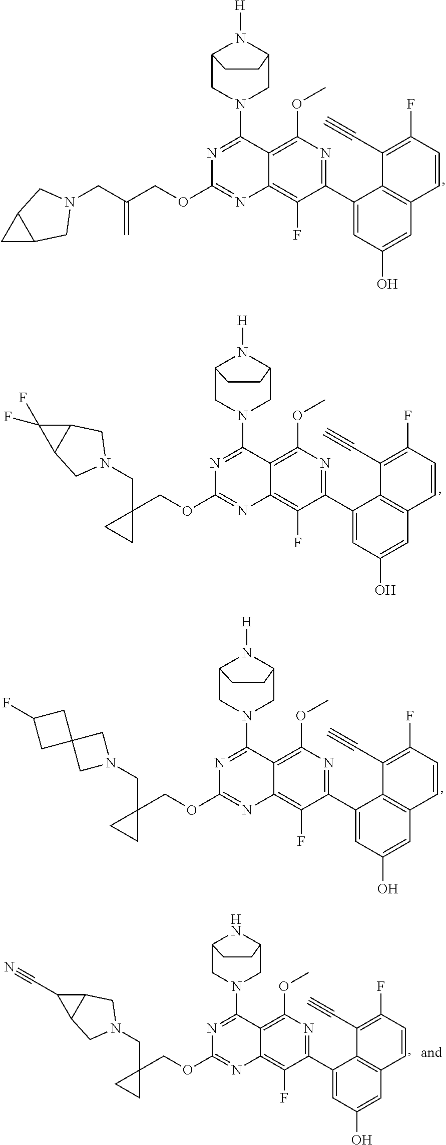 [SMILES: C#Cc1c(F)ccc2cc(O)cc(-c3nc(OC)c4c(N5CC6CCC(C5)N6)nc(OCC(=C)CN5CC6CC6C5)nc4c3F)c12 ; C#Cc1c(F)ccc2cc(O)cc(-c3nc(OC)c4c(N5CC6CCC(C5)N6)nc(OCC5(CN6CC7C(C6)C7(F)F)CC5)nc4c3F)c12 ; C#Cc1c(F)ccc2cc(O)cc(-c3nc(OC)c4c(N5CC6CCC(C5)N6)nc(OCC5(CN6CC7(CC(F)C7)C6)CC5)nc4c3F)c12 ; C#Cc1c(F)ccc2cc(O)cc(-c3nc(OC)c4c(N5CC6CCC(C5)N6)nc(OCC5(CN6CC7C(C#N)C7C6)CC5)nc4c3F)c12] 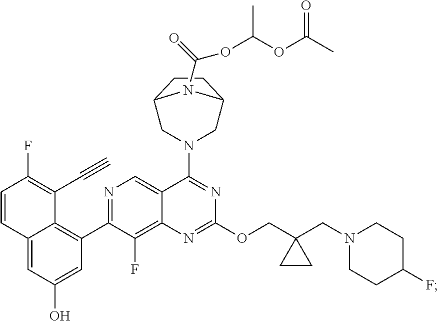 [SMILES: C#Cc1c(F)ccc2cc(O)cc(-c3ncc4c(N5CC6CCC(C5)N6C(=O)OC(C)OC(C)=O)nc(OCC5(CN6CCC(F)CC6)CC5)nc4c3F)c12]
or a pharmaceutically acceptable salt of any of the foregoing.

24. A pharmaceutical composition comprising a compound of any one of claims 1 to 23, or a pharmaceutically acceptable salt thereof; and a pharmaceutically acceptable carrier.

25. A method for treating cancer in a subject comprising administering to the subject and effective amount of a compound of any one of claims 1 to 23, or a pharmaceutically acceptable salt thereof, or the pharmaceutically acceptable composition of claim 24.
